(19) United States
(12) Patent Application Publication
(10) Pub. No.: US 2023/0138729 A1
(43) Pub. Date: May 4, 2023

(54) Coumarin Derivatives of Sugar Analogs and Uses Thereof

(71) Applicant: Rubedo Life Sciences, Inc., Sunnyvale, CA (US)

(72) Inventors: Paul Keitz, Redwood City, CA (US); Gus Bergnes, Pacifica, CA (US)

(21) Appl. No.: 17/943,836

(22) Filed: Sep. 13, 2022

Related U.S. Application Data
(60) Provisional application No. 63/243,544, filed on Sep. 13, 2021.

(51) Int. Cl. C12Q 1/40; C07H 15/207; C07H 17/075; C07H 15/26; C07H 17/04

(52) U.S. Cl. CPC C12Q 1/40; C07H 15/207; C07H 17/075; C07H 15/26; C07H 17/04

(57) ABSTRACT

Provided herein are coumarin derivatives of sugar analogs which are used to measure the rate of hydrolysis of these sugar analogs when contacted with a glycosidase. The reactivity of the coumarin derivatives serves as a convenient method for estimating for the rate of hydrolysis of sugar analogs when used a promoiety with cytotoxic drugs to generate senolytic agents with improved selectivity for killing senescent cells.

### CROSS REFERENCE TO RELATED APPLICATION

This application claims priority to U.S. Provisional Application Ser. No. 63/243,544 filed Sep. 13, 2021, under 35 U.S.C. § 119 (e) which is incorporated by reference in its entirety.

### TECHNICAL FIELD

Provided herein are coumarin derivatives of sugar analogs which are used to measure the rate of hydrolysis of these sugar analogs when contacted with a glycosidase. The reactivity of the coumarin derivatives serves as a convenient method for estimating for the rate of hydrolysis of sugar analogs when used a promoiety with cytotoxic drugs to generate senolytic agents with improved selectivity for killing senescent cells.

### BACKGROUND

Non-toxic prodrugs of senolytic agents, which are activated by glycosidases that preferentially accumulate inside senescent cells, are particularly effective agents for selectively killing senescent cells (Gallop et al., International Publication No. WO 2020/014409). In general, these prodrugs are cytotoxic agents (i.e., histone deacetylase inhibitors, Hsp90 inhibitors, topoisomerase 1 inhibitors, Bc12 inhibitors, etc.) conjugated with a sugar promoiety (i.e., a galactose or fucose analog).

New prodrugs of senolytic agents, which incorporate sugar analogs are being prepared to optimize for example, toxicity, permeability and bioavailability. However, synthesis of senolytic agents conjugated with novel sugar promoieties is a complex and laborious process. Accordingly, what is needed is a simple method for estimating whether the novel sugar promoieties are substrates for glycosidases found in senescent cells prior to preparing novel senolytic agents incorporating such promoieties.

### SUMMARY

The present invention satisfies these and other needs by providing coumarin derivatives of sugar analogs. The rate of hydrolysis of coumarin derivatives of sugar analogs when contacted with a glycosidase provides a convenient estimate of the rate of hydrolysis of senolytic prodrugs which incorporate these sugar analogs.

In one aspect, a compound of Formula (I) or Formula (II) or pharmaceutically available salts, hydrates and solvates thereof, is provided.

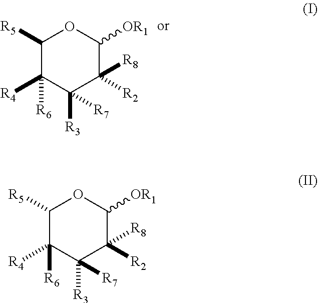

In a compound of Formula (I) or Formula (II), R₁ is

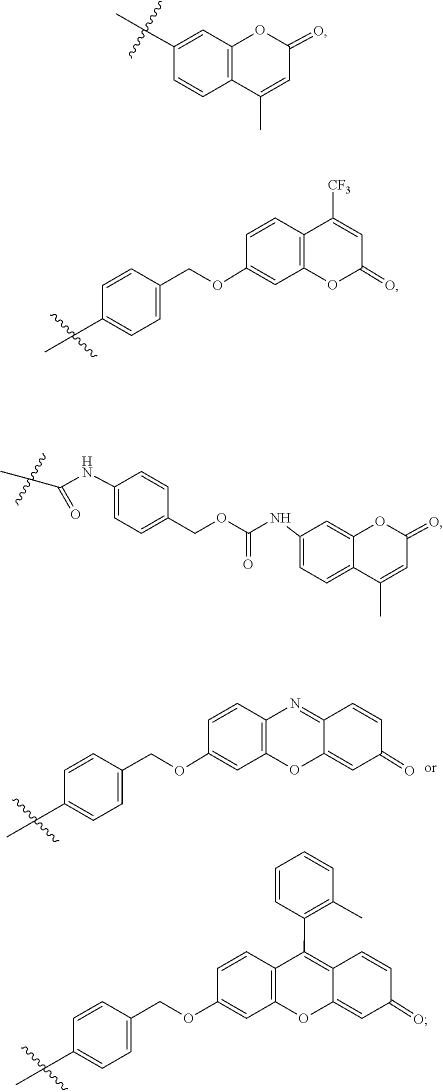 [SMILES: *c1ccc2c(C)cc(=O)oc2c1 ; *c1ccc(COc2ccc3c(C(F)(F)F)cc(=O)oc3c2)cc1 ; *C(=O)Nc1ccc(COC(=O)Nc2ccc3c(C)cc(=O)oc3c2)cc1 ; *c1ccc(COc2ccc3nc4ccc(=O)cc-4oc3c2)cc1 ; *c1ccc(COc2ccc3c(-c4ccccc4C)c4ccc(=O)cc-4oc3c2)cc1]

R₂ is —H, —F, —OH, —OC(O)R₉ or —OC(O)OR₁₀; R₃ is —H, —F, —OH, —OC(O)R₁₁ or —OC(O)OR₁₂; R₄ is —H, —F, —OH, —OC(O)R₁₃ or —OC(O)OR₁₄; alternatively, both R₃ and R₄ together with the atoms to which they are bonded form a 5 membered cyclic acetal which is substituted by R₁₇ at the acetal carbon atom; alternatively, both R₃ and R₄ together with the atoms to which they are bonded form a 5 membered cyclic carbonate; R₅ is —CH₃, —CH₂F, —CHF₂, —CF₃, —CH₂OH, —CH₂OC(O)R₁₅ or —CH₂OC(O)OR₁₆; R₆ is —H or —F; R₇ is —H or —F; R₈ is —H or —F; and R₉-R₁₇ are independently alkyl, substituted alkyl, alkenyl, substituted alkenyl, alkynyl, substituted alkynyl, aryl, substituted aryl, cycloalkyl, substituted cycloalkyl, cycloheteroalkyl, substituted cycloheteroalkyl, heteroaryl or substituted heteroaryl; provided that when R₅ is —CH₂F, —CHF₂ or —CF₃, then one of R₂, R₃ or R₄ is —H or —F; provided that when R₅ is —CH₃, —CH₂OH, —CH₂OC(O)R₁₅ or —CH₂OC(O)OR₁₆, then one or two of R₂, R₃ or R₄ is —H or —F; provided that R₆ is —F only if R₄ is —F; R₇ is —F only if R₃ is —F; and R₈ is —F only if R₂ is —F; and provided R₂ and R₄ are not —F and R₅ is not —CH₃.

In another aspect, a diagnostic composition is provided. The diagnostic composition includes a compound of Formula (I) or Formula (II) or pharmaceutically available salts, hydrates and solvates and a diagnostically acceptable vehicle.

In still another aspect, a method of measuring the rate of hydrolysis of a compound of Formula (I) or Formula (II) or pharmaceutically available salts, hydrates and solvates is provided. The method includes adding a glycosidase to a diagnostic composition. In some embodiments, the glycosidase is a galactosidase or a fucosidase.

### DETAILED DESCRIPTION

### Definitions

Unless defined otherwise, all technical and scientific terms used herein have the same meaning as is commonly understood by one of ordinary skill in the art to which this invention belongs. If a plurality of definitions for a term exist herein, those in this section prevail unless stated otherwise.

As used herein, and unless otherwise specified, the terms “about” and “approximately,” when used in connection with a property with a numeric value or range of values indicate that the value or range of values may deviate to an extent deemed reasonable to one of ordinary skill in the art while still describing the particular property. Specifically, the terms “about” and “approximately,” when used in this context, indicate that the numeric value or range of values may vary by 5%, 4%, 3%, 2%, 1%, 0.9%, 0.8%, 0.7%, 0.6%, 0.5%, 0.4%, 0.3%, 0.2% or 0.1% of the recited value or range of values. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line drawn through a line in a structure indicates a point of attachment of a group. Unless chemically or structurally required, no directionality is indicated or implied by the order in which a chemical group is written or named.

The prefix “Cᵤ₋ᵥ” indicates that the following group has from u to v carbon atoms. It should be understood that u to v carbons includes u+1 to v, u+2 to v, u+3+v, etc. carbons, u+1 to u+3 to v, u+1 to u+4 to v, u+2 to u+4 to v, etc. and cover all possible permutation of u and v.

“Alkyl,” by itself or as part of another substituent, refers to a saturated or unsaturated, branched, straight-chain or cyclic monovalent hydrocarbon radical derived by the removal of one hydrogen atom from a single carbon atom of a parent alkane. Typical alkyl groups include, but are not limited to, methyl; ethyl; propyls such as propan-1-yl, propan-2-yl, etc.; butyls such as butan-1-yl, butan-2-yl, 2-methyl-propan-1-yl, 2-methyl-propan-2-yl, etc.; and the like. In some embodiments, an alkyl group comprises from 1 to 20 carbon atoms (C₁-C₂₀ alkyl). In other embodiments, an alkyl group comprises from 1 to 10 carbon atoms (C₁-C₁₀ alkyl). In still other embodiments, an alkyl group comprises from 1 to 6 carbon atoms (C₁-C₆ alkyl).

“Alkenyl,” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl ; cycloprop-2-en-1-yl ; butenyls such as but-1-en-1-yl, but-1 -en-2-yl, 2-methyl-prop-1 -en-1 -yl, but-2-en-1-yl , but-2-en-1-yl, but-2-en-2-yl, buta-1,3-dien-1-yl, buta-1,3-di en-2-yl, cyclobut-1-en-1-yl, cyclobut-1-en-3 -yl, cyclobuta-1,3-dien-1-yl, etc.; and the like. In some embodiments, an alkenyl group comprises from 1 to 20 carbon atoms (C₁-C₂₀ alkenyl). Inn other embodiments, an alkenyl group comprises from 1 to 10 carbon atoms (C₁-C₁₀ alkenyl). In still other embodiments, an alkenyl group comprises from 1 to 6 carbon atoms (C₁-C₆ alkenyl).

“Alkynyl,” by itself or as part of another substituent refers to an unsaturated branched, straight-chain or cyclic alkyl radical having at least one carbon-carbon triple bond derived by the removal of one hydrogen atom from a single carbon atom of a parent alkyne. Typical alkynyl groups include, but are not limited to, ethynyl; propynyls such as prop-1-yn-1-yl, prop-2-yn-1-yl, etc.; butynyls such as but-1-yn-1-yl, but-1-yln-3-yl, but-3-yn-1-yl, etc.; and the like. In some embodiments, an alkynyl group comprises from 1 to 20 carbon atoms (C₁-C₂₀ alkynyl). In other embodiments, an alkynyl group comprises from 1 to 10 carbon atoms (C₁-C₁₀ alkynyl). In still other embodiments, an alkynyl group comprises from 1 to 6 carbon atoms (C₁-C₆ alkynyl).

“Aryl,” by itself or as part of another substituent, refers to a monovalent aromatic hydrocarbon group derived by the removal of one hydrogen atom from a single carbon atom of a parent aromatic ring system, as defined herein. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In some embodiments, an aryl group comprises from 6 to 20 carbon atoms (C₆-C₂₀ aryl). In other embodiments, an aryl group comprises from 6 to 15 carbon atoms (C₆-C₁₅ aryl). In still other embodiments, an aryl group comprises from 6 to 10 carbon atoms (C₆-C₁₀ aryl).

“Arylalkyl,” by itself or as part of another substituent, refers to an acyclic alkyl group in which one of the hydrogen atoms bonded to a carbon atom, typically a terminal or sp³ carbon atom, is replaced with an aryl group as, as defined herein. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. In some embodiments, an arylalkyl group is (C₆-C₃₀) arylalkyl, e.g., the alkyl moiety of the arylalkyl group is (C₁-C₁₀) alkyl and the aryl moiety is (C₆-C₂₀) aryl. In other embodiments, an arylalkyl group is (C₆-C₂₀) arylalkyl, e.g., the alkyl moiety of the arylalkyl group is (C₁-C₈) alkyl and the aryl moiety is (C₆-C₁₂) aryl. In still other embodiments, an arylalkyl group is (C₆-C₁₅) arylalkyl, e.g., the alkyl moiety of the arylalkyl group is (C₁-C₅) alkyl and the aryl moiety is (C₆-C₁₀) aryl.

“Arylalkenyl,” by itself or as part of another substituent, refers to an acyclic alkenyl group in which one of the hydrogen atoms bonded to a carbon atom, is replaced with an aryl group as, as defined herein. In some embodiments, an arylalkenyl group is (C₆-C₃₀) arylalkenyl, e.g., the alkenyl moiety of the arylalkenyl group is (C1-C10) alkenyl and the aryl moiety is (C₆-C₂₀) aryl. In other embodiments, an arylalkenyl group is (C₆-C₂₀) arylalkenyl, e.g., the alkenyl moiety of the arylalkenyl group is (C₁-C₈) alkenyl and the aryl moiety is (C₆-C₁₂) aryl. In still other embodiments, an arylalkenyl group is (C₆-C₁₅) arylalkenyl, e.g., the alkenyl moiety of the arylalkenyl group is (C₁-C₅) alkenyl and the aryl moiety is (C₆-C₁₀) aryl.

“Arylalkynyl,” by itself or as part of another substituent, refers to an acyclic alkynyl group in which one of the hydrogen atoms bonded to a carbon atom, is replaced with an aryl group as, as defined herein. In some embodiments, an arylalkynyl group is (C₆-C₃₀) arylalkynyl, e.g., the alkynyl moiety of the arylalkynyl group is (C₁-C₁₀) alkynyl and the aryl moiety is (C₆-C₂₀) aryl. In other embodiments, an arylalkynyl group is (C₆-C₂₀) arylalkynyl, e.g., the alkynyl moiety of the arylalkenyl group is (C₁-C₈) alkynyl and the aryl moiety is (C₆-C₁₂) aryl. In still other embodiments, an arylalkynyl group is (C₆-C₁₅) arylalkynyl, e.g., the alkynyl moiety of the arylalkynyl group is (C₁-C₅) alkynyl and the aryl moiety is (C₆-C₁₀) aryl.

“Cycloalkyl,” by itself or as part of another substituent, refers to a saturated cyclic monovalent hydrocarbon radical derived by the removal of one hydrogen atom from a single carbon atom of a parent cycloalkane. Typical cycloalkyl groups include, but are not limited to, cyclopropyl, cyclobutyl, cyclopentyl cycopentenyl; etc.; and the like. In some embodiments, a cycloalkyl group comprises from 3 to 20 carbon atoms (C₁-C₁₅ cycloalkyl). In other embodiments, a cycloalkyl group comprises from 3 to 10 carbon atoms (C₁-C₁₀ cycloalkyl). In still other embodiments, a cycloalkyl group comprises from 3 to 8 carbon atoms (C₁-C₈ cycloalkyl). The term “cyclic monovalent hydrocarbon radical” also includes multicyclic hydrocarbon ring systems having a single radical and between 3 and 12 carbon atoms. Exemplary multicyclic cycloalkyl rings include, for example, norbornyl, pinyl, and adamantyl.

“Cycloalkenyl,” by itself or as part of another substituent, refers to an unsaturated cyclic monovalent hydrocarbon radical derived by the removal of one hydrogen atom from a single carbon atom of a parent cycloalkene. Typical cycloalkenyl groups include, but are not limited to, cyclopropene, cyclobutene cyclopentene; etc.; and the like. In some embodiments, a cycloalkenyl group comprises from 3 to 20 carbon atoms (C₁-C₂₀ cycloalkenyl). In other embodiments, a cycloalkenyl group comprises from 3 to 10 carbon atoms (C₁-C₁₀ cycloalkenyl). In still other embodiments, a cycloalkenyl group comprises from 3 to 8 carbon atoms (C₁-C₈ cycloalkenyl). The term ‘cyclic monovalent hydrocarbon radical” also includes multicyclic hydrocarbon ring systems having a single radical and between 3 and 12 carbon atoms.

“Cycloheteroalkyl,” by itself or as part of another substituent, refers to a cycloalkyl group as defined herein in which one or more one or more of the carbon atoms (and optionally any associated hydrogen atoms), are each, independently of one another, replaced with the same or different heteroatoms or heteroatomic groups as defined in “heteroalkyl” below. In some embodiments, a cycloheteroalkyl group comprises from 3 to 20 carbon and hetero atoms (₁₋₂₀ cycloheteroalkyl). In other embodiments, a cycloheteroalkyl group comprises from 3 to 10 carbon and hetero atoms (₁₋₁₀ cycloheteroalkyl). In still other embodiments, a cycloheteroalkyl group comprises from 3 to 8 carbon and hetero atoms (₁₋₈ cycloheteroalkyl). The term “cyclic monovalent heteroalkyl radical” also includes multicyclic heteroalkyl ring systems having a single radical and between 3 and 12 carbon and at least one hetero atom.

“Cycloheteroalkenyl,” by itself or as part of another substituent, refers to a cycloalkenyl group as defined herein in which one or more one or more of the carbon atoms (and optionally any associated hydrogen atoms), are each, independently of one another, replaced with the same or different heteroatoms or heteroatomic groups as defined in “heteroalkenyl” below. In some embodiments, a cycloheteroalkenyl group comprises from 3 to 20 carbon and hetero atoms (₁₋₂₀ cycloheteroalkenyl). In other embodiments, a cycloheteroalkenyl group comprises from 3 to 10 carbon and hetero atoms (₁₋₁₀₎ cycloheteroalkenyl). In still other embodiments, a cycloheteroalkenyl group comprises from 3 to 8 carbon and heteroatoms (₁₋₈ cycloheteroalkenyl). The term “cyclic monovalent heteroalkenyl radical” also includes multicyclic heteroalkenyl ring systems having a single radical and between 3 and 12 carbon and at least one hetero atoms.

“Compounds,” refers to compounds encompassed by structural formulae disclosed herein and includes any specific compounds within these formulae whose structure is disclosed herein. Compounds may be identified either by their chemical structure and/or chemical name. The chemical structure is determinative of the identity of the compound. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass the stereoisomerically pure form depicted in the structure (e.g., geometrically pure, enantiomerically pure or diastereomerically pure). The chemical structures depicted herein also encompass the enantiomeric and stereoisomeric derivatives of the compound depicted. Enantiomeric and stereoisomeric mixtures can be resolved into their component enantiomers or stereoisomers using separation techniques or chiral synthesis techniques well known to the skilled artisan. The compounds may also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that may be incorporated into the compounds disclosed herein include, but are not limited to ²H, ³H, ¹¹C, ¹³C, ¹⁴C, ¹⁵N, ¹⁸O, ¹⁷O, etc. Compounds may exist in unsolvated forms as well as solvated forms, including hydrated forms. In general, compounds may be hydrated or solvated. Certain compounds may exist in multiple crystalline or amorphous forms. In general, all physical forms are equivalent for the uses contemplated herein and are intended to be within the scope of the present disclosure. Further, it should be understood, when partial structures of the compounds are illustrated, that brackets indicate the point of attachment of the partial structure to the rest of the molecule.

“Diagnostically effective amount,” means the amount of a compound that is capable of being detected. The “diagnostically effective amount” will vary depending on the compound.

“Halo,” by itself or as part of another substituent refers to a radical —F, —Cl, —Br or —I.

“Heteroalkyl,” refer to an alkyl, group, in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O —, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SnR⁵⁰⁷R⁵⁰⁸ and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some embodiments, an heteroalkyl group comprises from 1 to 20 carbon and hetero atoms (₁₋₂₀ heteroalkyl). In other embodiments, an heteroalkyl group comprises from 1 to 10 carbon and hetero atoms (₁₋₁₀ heteroalkyl). In still other embodiments, an heteroalkyl group comprises from 1 to 6 carbon and hetero atoms (₁₋₆ heteroalkyl).

“Heteroalkenyl,” refers to an alkenyl group in which one or more of the carbon atoms (and optionally any associated hydrogen atoms), are each, independently of one another, replaced with the same or different heteroatoms or heteroatomic groups. Typical heteroatoms or heteroatomic groups which can replace the carbon atoms include, but are not limited to, —O—, —S—, —N—, —Si—, —NH—, —S(O)—, —S(O)₂—, —S(O)NH—, —S(O)₂NH— and the like and combinations thereof. The heteroatoms or heteroatomic groups may be placed at any interior position of the alkyl, alkenyl or alkynyl groups. Typical heteroatomic groups which can be included in these groups include, but are not limited to, —O—, —S—, —O—O—, —S—S—, —O—S—, —NR⁵⁰¹R⁵⁰², ═N—N═, —N═N—, —N═N—NR⁵⁰³R⁴⁰⁴, —PR⁵⁰⁵—, —P(O)₂—, —POR⁵⁰⁶—, —O—P(O)₂—, —SO—, —SO₂—, —SNR⁵⁰⁷R⁵⁰⁸and the like, where R⁵⁰¹, R⁵⁰², R⁵⁰³, R⁵⁰⁴, R⁵⁰⁵, R⁵⁰⁶, R⁵⁰⁷ and R⁵⁰⁸ are independently hydrogen, alkyl, aryl, substituted aryl, heteroalkyl, heteroaryl or substituted heteroaryl. In some embodiments, an heteroalkenyl group comprises from 1 to 20 carbon and hetero atoms (₁₋₂₀ heteroalkenyl). In other embodiments, an heteroalkenyl group comprises from 1 to 10 carbon and hetero atoms (₁₋₁₀ heteroalkenyl). In still other embodiments, an heteroalkenyl group comprises from 1 to 6 carbon and hetero atoms (₁₋₆ heteroalkenyl).

“Heteroaryl,” by itself or as part of another substituent, refers to a monovalent heteroaromatic radical derived by the removal of one hydrogen atom from a single atom of a parent heteroaromatic ring systems, as defined herein. Typical heteroaryl groups include, but are not limited to, groups derived from acridine, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In some embodiments, the heteroaryl group comprises from 5 to 20 ring atoms (5-20 membered heteroaryl). In other embodiments, the heteroaryl group comprises from 5 to 10 ring atoms (5-10 membered heteroaryl). Exemplary heteroaryl groups include those derived from furan, thiophene, pyrrole, benzothiophene, benzofuran, benzimidazole, indole, pyridine, pyrazole, quinoline, imidazole, oxazole, isoxazole and pyrazine.

“Heteroarylalkyl,” by itself or as part of another substituent refers to an acyclic alkyl group in which one of the hydrogen atoms bonded to a carbon atom, typically a terminal or sp³ carbon atom, is replaced with a heteroaryl group. In some embodiments, the heteroarylalkyl group is a 6-21 membered heteroarylalkyl, e.g., the alkyl moiety of the heteroarylalkyl is (C₁-C₆) alkyl and the heteroaryl moiety is a 5-15-membered heteroaryl. In other embodiments, the heteroarylalkyl is a 6-13 membered heteroarylalkyl, e.g., the alkyl moiety is (C₁-C₃) alkyl and the heteroaryl moiety is a 5-10 membered heteroaryl.

“Heteroarylalkenyl,” by itself or as part of another substituent refers to an acyclic alkenyl group in which one of the hydrogen atoms bonded to a carbon atom, is replaced with a heteroaryl group. In some embodiments, the heteroarylalkenyl group is a 6-21 membered heteroarylalkyl, e.g., the alkenyl moiety of the heteroarylalkenyl is (C₁-C₆) alkenyl and the heteroaryl moiety is a 5-15-membered heteroaryl. In other embodiments, the heteroarylalkenyl is a 6-13 membered heteroarylalkenyl, e.g., the alkenyl moiety is (C₁-C₃) alkyl and the heteroaryl moiety is a 5-10 membered heteroaryl.

“Heteroarylalkynyl,” by itself or as part of another substituent refers to an acyclic alkenyl group in which one of the hydrogen atoms bonded to a carbon atom, is replaced with a heteroaryl group. In some embodiments, the heteroarylalkynyl group is a 6-21 membered heteroarylalkyl, e.g., the alkynyl moiety of the heteroarylalkynyl is (C₁-C₆) alkynyl and the heteroaryl moiety is a 5-15-membered heteroaryl. In other embodiments, the heteroarylalkynyl is a 6-13 membered heteroarylalkynyl, e.g., the alkynyl moiety is (C₁-C₃) alkyl and the heteroaryl moiety is a 5-10 membered heteroaryl.

“Hydrates,” refers to incorporation of water into to the crystal lattice of a compound described herein, in stoichiometric proportions, resulting in the formation of an adduct. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. See e.g., (Guillory, K., Chapter 5, pp. 202205 in Polymorphism in Pharmaceutical Solids, (Brittain, H. ed.), Marcel Dekker, Inc., New York, N.Y., 1999). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205208 in Polymorphism in Pharmaceutical Solids, (Brittain, H. ed.), Marcel Dekker, Inc. New York, 1999). In addition, many commercial companies routinely offer services that include preparation and/or characterization of hydrates such as, for example, HOLODIAG, Pharmaparc II, Voie de l'Innovation, 27 100 Val de Reuil, France (http://www.holodiag.com).

“Parent Aromatic Ring System,” refers to an unsaturated cyclic or polycyclic ring system having a conjugated p electron system. Specifically included within the definition of “parent aromatic ring system” are fused ring systems in which one or more of the rings are aromatic and one or more of the rings are saturated or unsaturated, such as, for example, fluorene, indane, indene, phenalene, etc. Typical parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Parent Heteroaromatic Ring System,” refers to a parent aromatic ring system in which one or more carbon atoms (and optionally any associated hydrogen atoms) are each independently replaced with the same or different heteroatom. Typical heteroatoms to replace the carbon atoms include, but are not limited to, N, P, O, S, Si, etc. Specifically included within the definition of “parent heteroaromatic ring system” are fused ring systems in which one or more of the rings are aromatic and one or more of the rings are saturated or unsaturated, such as, for example, benzodioxan, benzofuran, chromane, chromene, indole, indoline, xanthene, etc. Typical parent heteroaromatic ring systems include, but are not limited to, arsindole, carbazole, b-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Pharmaceutically acceptable salt,” refers to a salt of a compound which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Prodrug” as used herein, refers to a derivative of a drug molecule that requires a transformation within the body to release the active drug. Prodrugs are frequently, although not necessarily, pharmacologically inactive until converted to the parent drug.

“Promoiety” as used herein, refers to a form of protecting group that when used to mask a functional group within a drug molecule converts the drug into a prodrug. Typically, the promoiety will be attached to the drug via bond(s) that are cleaved by enzymatic or non-enzymatic means in vivo.

“Protecting group,” refers to a grouping of atoms that when attached to a reactive functional group in a molecule masks, reduces or prevents reactivity of the functional group during chemical synthesis. Examples of protecting groups can be found in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996). Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilyl-ethanesulfonyl (“SES”), trityl and substituted trityl groups, allyloxycarbonyl, 9-fluorenylmethyloxycarbonyl (“FMOC”), nitro-veratryloxycarbonyl (“NVOC”) and the like. Representative hydroxy protecting groups include, but are not limited to, those where the hydroxy group is either acylated or alkylated such as benzyl, and trityl ethers as well as alkyl ethers, tetrahydropyranyl ethers, trialkylsilyl ethers and allyl ethers.

“Solvates,” refers to incorporation of solvents into to the crystal lattice of a compound described herein, in stoichiometric proportions, resulting in the formation of an adduct. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. See e.g., (Guillory, K., Chapter 5, pp. 205208 in Polymorphism in Pharmaceutical Solids, (Brittain, H. ed.), Marcel Dekker, Inc., New York, N.Y., 1999)). The above methods for preparing solvates are well within the ambit of those of skill in the art, are completely conventional and do not require any experimentation beyond what is typical in the art. Solvates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205208 in Polymorphism in Pharmaceutical Solids, (Brittain, H. ed.), Marcel Dekker, Inc. New York, 1999). In addition, many commercial companies routine offer services that include preparation and/or characterization of solvates such as, for example, HOLODIAG, Pharmaparc II, Voie de l'Innovation, 27 100 Val de Reuil, France (http.//www.holodiag.com).

“Substituted,” when used to modify a specified group or radical, means that one or more hydrogen atoms of the specified group or radical are each, independently of one another, replaced with the same or different substituent(s). Substituent groups useful for substituting saturated carbon atoms in the specified group or radical include IV, halo, —O⁻, ═O, —OR^{b}, —SR^{b}, —S⁻, ═S, —NR^{c}R^{c}, ═NR^{b}, ═N—OR^{b}, trihalomethyl, —CF₃, —CN, —OCN, —SCN, —NO, —NO₂, —N—OR^{b}, —N—NR^{c}R^{c}, —NR^{b}S(O)₂R^{b}, ═N₂, —N₃, —S(O)₂R^{b}, —S(O)₂NR^{b}R^{b}, —S(O)₂O⁻, —S(O)₂OR^{b}, —OS(O)₂R^{b}, —OS(O)₂O⁻, —OS(O)₂OR^{b}, —OS(O)₂NR^{c}NR^{c}, —P(O)(O⁻)₂, —P(O)(OR^{b})(O⁻), —P(O)(OR^{b})(OR^{b}), —C(O)R^{b}, —C(O)NR^{b}—OR^{b} —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, OC(O)OR^{b}, —OC(O)NR^{c}R^{c}, —OC(NCN)NR^{c}R^{c} —OC(S)OR^{b}, —NR^{b}C(O)R^{b}, —NR^{b}C(S)R^{b}, —NR^{b}C(O)O⁻, —NR^{b}C(O)OR^{b}, —NR^{b}C(NCN)OR^{b}, —NR^{b}S(O)₂NR^{c}R^{c}, —NR^{b}C(S)OR^{b}, —NR^{b}C(O)NR^{c}R^{c}, —NR^{b}C(S)NR^{c}R^{c}, —NR^{b}C(S)NR^{b}C(O)R^{a}, —NR^{b}S(O)₂OR^{b}, —NR^{b}S(O)₂R^{b}, —NR^{b}S(O)₂R^{b}, —NR^{b}C(NCN)NR^{c}R^{c}, —NR^{b}C(NR^{b})R^{b} and —NR^{b}C(NR^{b})NR^{c}R^{c}, where each R^{a} is independently, aryl, substituted aryl, heteroalkyl, substituted heteroalkyl, heteroaryl or substituted heteroaryl; each R^{b} is independently hydrogen, alkyl, heteroalkyl, substituted heteroalkyl, arylalkyl, substituted arylalkyl, heteroarylalkyl or substituted heteroarylalkyl; and each R^{c} is independently R^{b} or alternatively, the two R^{c}s taken together with the nitrogen atom to which they are bonded form a 4-, 5-, 6- or 7 membered- cycloheteroalkyl, substituted cycloheteroalkyl or a cycloheteroalkyl fused with an aryl group which may optionally include from 1 to 4 of the same or different additional heteroatoms selected from the group consisting of O, N and S. As specific examples, —NR^{c}R^{c} is meant to include —NH₂, —NH-alkyl, N-pyrrolidinyl and N-morpholinyl. In other embodiments, substituent groups useful for substituting saturated carbon atoms in the specified group or radical include R^{a}, halo, —OR^{b}, —NR^{c}R^{c}, trihalomethyl, —CN, —NR^{b}S(O)₂R^{b}, —C(O)R^{b}, —C(O)NR^{b}—OR^{b}, —C(O)OR^{b}, —C(O)NR^{c}R^{c}, —OC(O)R^{b}, —OC(O)OR^{b}, —OS(O)₂NR^{c}NR^{c}, —OC(O)NR^{c}R^{c}, and —NR^{b}C(O)OR^{b}, where each R^{a} is independently alkyl, aryl, heteroaryl, each R^{b} is independently hydrogen, R^{a,} heteroalkyl, arylalkyl, heteroarylalkyl; and each R^{c} is independently R^{b} or alternatively, the two R^{c}s taken together with the nitrogen atom to which they are bonded form a 4-, 5-, 6 or -7 membered- cycloheteroalkyl ring.

Substituent groups useful for substituting unsaturated carbon atoms in the specified group or radical include —R^{a}, halo, —O⁻, —OR^{b}, —SR^{b}, —S-, —NR^{c}R^{c}, trihalomethyl, —CF₃, —CN, —OCN, —SCN, —NO, —NO₂, —N₃, —S(O)₂O- , —S(O)₂OR^{b}, —OS(O)₂R^{b}, —OS(O)₂OR^{b}, —OS(O)₂O⁻, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)O⁻, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)O⁻, —OC(O)OR^{b}, —OC(S)OR^{b}, —OC(O)NR^{c}R^{c}, —OS(O₂NR^{c}NR^{c}, —NR^{b}C(O)R^{b}, —NR^{b}C(S)R^{b}, —NR^{b}C(O)O⁻, —NR^{b}C(O)OR^{b}, —NR^{b}S(O)₂OR^{a}, —NR^{b}S(O)₂R^{a}, —NR^{b}C(S)OR^{b}, —NR^{b}C(O)NR^{c}R^{c}, —NR^{b}C(NR^{b})R^{b} and —NR^{b}C(NR^{b})NR^{c}R^{c} , where R^{a}, R^{b} and R^{c} are as previously defined. In other embodiments, substituent groups useful for substituting unsaturated carbon atoms in the specified group or radical include —R^{a}, halo, —OR^{b}, —SR^{b}, —NR^{c}R^{c}, trihalomethyl, —CN, —S(O)₂OR^{b}, —C(O)R^{b}, —C(O)OR^{b}, —C(O)NR^{c}R^{c}, —OC(O)R^{b}, —OC(O)R^{b}, —OS(O)₂NR^{c}NR^{c}, —NR^{b}C(O)R^{b} and —NR^{b}C(O)OR^{b}, where R^{a}, R^{b} and R^{c} are as previously defined.

Substituent groups useful for substituting nitrogen atoms in heteroalkyl and cycloheteroalkyl groups include, but are not limited to, —R^{a}, —O⁻, —OR^{b}, —SR^{b}, —S⁻, —NR^{c}R^{c}, trihalomethyl, —CF₃, —CN, —NO, —NO₂, —S(O)₂R^{b}, —S(O)₂ O⁻, —S(O)₂OR^{b}, —OS(O)₂R^{b}, —OS(O)₂O⁻, —OS(O)₂OR^{b}, —P(O)(O⁻)₂, —P(O)(OR^{b})(O⁻), —P(O)(OR^{b})(OR^{b}), —C(O)R^{b}, —C(S)R^{b}, —C(NR^{b})R^{b}, —C(O)OR^{b}, —C(S)OR^{b}, —C(O)NR^{c}R^{c}, —C(NR^{b})NR^{c}R^{c}, —OC(O)R^{b}, —OC(S)R^{b}, —OC(O)OR^{b}, —OC(S)OR^{b}, —NR^{b}C(O)R^{b}, —NR^{b}C(S)R^{b}, —NR^{b}C(O)OR^{b}, —NR^{b}C(S)OR^{b}, —NR^{b}C(O)NR^{c}R^{c}, —NR^{b}C(NR^{b})R^{b} and —NR^{b}C(NR^{b})NR^{c}R^{c}, where R^{a}, R^{b} R^{a} are as previously defined. In some embodiments, substituent groups useful for substituting nitrogen atoms in heteroalkyl and cycloheteroalkyl groups include, R^{a}, halo, —OR^{b}, —NR^{c}R^{c}, trihalomethyl, —CN, —S(O)₂OR^{b}, —OS(O)₂R^{b}, —OS(O)₂OR^{b}, —C(O)R^{b}, —C(NR^{b})R^{b}, —C(O)OR^{b}, —C(O)NR^{c}R^{c}, —OC(O)R^{b}, —OC(O)OR^{b}, —OS(O)₂NR^{c}NR^{c}, —NR^{b}C(O)R^{b} and —NR^{b}C(O)OR^{b}, where R^{a}, R^{b} and R^{c} are as previously defined.

Substituent groups from the above lists useful for substituting other specified groups or atoms will be apparent to those of skill in the art.

The substituents used to substitute a specified group can be further substituted, typically with one or more of the same or different groups selected from the various groups specified above.

“Subject,” “individual,” or “patient,” is used interchangeably herein and refers to a vertebrate, preferably a mammal. Mammals include, but are not limited to, murines, rodents, simians, humans, farm animals, sport animals and pets.

“Vehicle,” refers to a diluent, excipient or carrier with which a compound is administered to a subject. In some embodiments, the vehicle is pharmaceutically acceptable.

Reference will now be made in detail to particular embodiments of compounds and methods. The disclosed embodiments are not intended to be limiting of the claims. To the contrary, the claims are intended to cover all alternatives, modifications and equivalents.

### COMPOUNDS

Provided herein are coumarin derivatives of sugar analogs.

In some embodiments, compounds of Formula (III) or Formula (IV) or pharmaceutically available salts, hydrate and solvates thereof are provided where

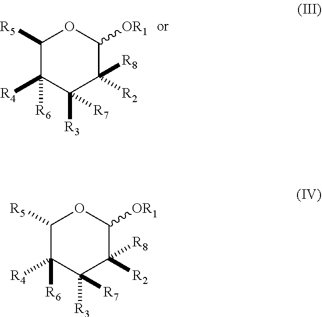

### R₁ is

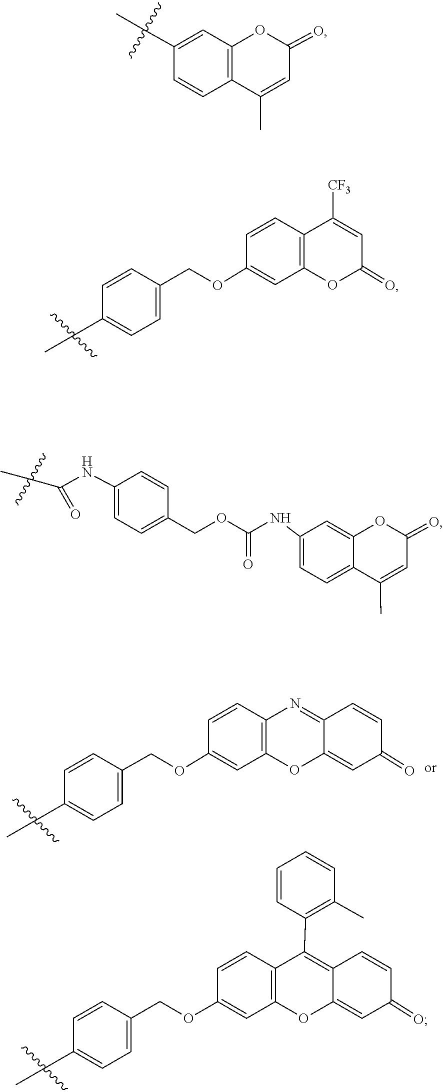 [SMILES: *c1ccc2c(C)cc(=O)oc2c1 ; *c1ccc(COc2ccc3c(C(F)(F)F)cc(=O)oc3c2)cc1 ; *C(=O)Nc1ccc(COC(=O)Nc2ccc3c(C)cc(=O)oc3c2)cc1 ; *c1ccc(COc2ccc3nc4ccc(=O)cc-4oc3c2)cc1 ; *c1ccc(COc2ccc3c(-c4ccccc4C)c4ccc(=O)cc-4oc3c2)cc1]

R₂ is —H, —F, —OH, —OC(O)R₉ or —OC(O)OR₁₀; R₃ is —H, —F, —OH, —OC(O)R₁₁ or —OC(O)OR₁₂; R₄ is —H, —F, —OH, —OC(O)R₁₃ or —OC(O)OR_{14;} alternatively, both R₃ and R₄ together with the atoms to which they are bonded form a 5 membered cyclic acetal which is substituted by R₁₇ at the acetal carbon atom; alternatively, both R₃ and R₄ together with the atoms to which they are bonded form a 5 membered cyclic carbonate; R₅ is —CH₃, —CH₂F, —CHF₂, —CF₃, —CH₂OH, —CH₂OC(O)R₁₅ or —CH₂OC(O)OR₁₆; R₆ is —H or —F; R₇ is —H or —F; R₈ is —H or —F; and R₉-R₁₇ are independently alkyl, substituted alkyl, alkenyl, substituted alkenyl, alkynyl, substituted alkynyl, aryl, substituted aryl, cycloalkyl, substituted cycloalkyl, cycloheteroalkyl, substituted cycloheteroalkyl, heteroaryl or substituted heteroaryl; provided that when R₅ is —CH₂F, —CHF₂ or —CF₃, then one of R₂, R₃ or R₄ is —H or —F; provided that when R₅ is —CH₃, —CH₂OH, —CH₂OC(O)R₁₅ or —CH₂OC(O)OR₁₆, then one or two of R₂, R₃ or R₄ is —H or —F; and provided that R₆ is —F only if R₄ is —F; R₇ is —F only if R₃ is —F; and R₈ is —F only if R₂ is —F.

In some embodiments, R₅ is —CH₃ and R₂ is —H or —F. In other embodiments, R₅ is —CH₃ and R₃ is —H or —F. In still other embodiments, R₅ is —CH₃ and R₄ is —H or —F. In still other embodiments, R₅ is —CH₃, R₂ is —F and R₈ is —F. In still other embodiments, R₅ is —CH₃, R₃ is —F and R₇ is —F. In still other embodiments, R₅ is —CH₃, R₄ is —F and R₆ is —F.

In some embodiments, R₅ is —CH₂OH, —CH₂OC(O)R₁₅ or —CH₂OC(O)OR₁₆ and R₂ is —H or —F. In other embodiments, R₅ is —CH₂OH, —CH₂OC(O)R₁₅ or —CH₂OC(O)OR₁₆ and R₃ is —H or —F. In still other embodiments, R₅ is —CH₂OH, —CH₂OC(O)R₁₅ or —CH₂OC(O)ORi₁₆ and R₄ is —H or —F. In still other embodiments, R₅ is —CH₂OH, —CH₂OC(O)R₁₅ or —CH₂OC(O)OR₁₆, R₂ is —F and R₈ is —F. In still other embodiments, R₅ is —CH₂OH, —CH₂OC(O)R₁₅ or —CH₂OC(O)OR₁₆, R₃ is —F and R₇ is —F. In still other embodiments, R₅ is —CH₂OH, —CH₂OC(O)R₁₅ or —CH₂OC(O)OR₁₆, R₄ is —F and R₆ is —F.

In some embodiments, R₅ is —CH₂F, —CHF₂ or —CF₃ and R₂ is —H or —F. In other embodiments, R₅ is —CH₂F, —CHF₂ or —CF₃ and R₃ is —H or —F. In still other embodiments, R₅ is —CH₂F, —CHF₂ or —CF₃ and R₄ is —H or —F. In still other embodiments, R₅ is —CH₂F, —CHF₂ or —CF₃, R₂ is —F and R₈ is —F. In still other embodiments, R₅ is —CH₂F, —CHF₂ or —CF₃, R₃ is —F and R₇ is —F. In still other embodiments, R₅ is —CH₂F, —CHF₂ or —CF₃, R₄ is —F and R₆ is —F.

In some embodiments, R₂ is —H or —F and R₃ is —H or —F. In other embodiments, R₂ is —H or —F and R₄ is —H or —F. In still other embodiments, R₃ is —H or —F and R₄ is —H or —F. In still other embodiments, R₂ is —H or —F, R₃ is —F and R₇ is —F. In still other embodiments, R₂ is —H or —F, R₄ is —F and R₆ is —F. In still other embodiments, R₃ is —H or —F, R₄ is —F and R₆ is —F. In still other embodiments, R₂ is —F, R₈ is —F and R₃ is —H or —F. In still other embodiments, R₂ is —F, R₈ is —F and R₄ is —H or —F. In still other embodiments, R₃ is —F, R₇ is —F and R₄ is —H or —F. In still other embodiments, R₃ is —F, R₇ is —F and R₂ is —H or —F.

In some embodiments, R₂ is —F and R₈ is —F. In other embodiments, R₃ is —F and R₇ is —F. In still other embodiments, R₄ is —F and R₆ is —F.

In some embodiments, R₂ is —H or —F. In some other embodiments, R₃ is —H or —F.

In still other embodiments, R₄ is —H or —F.

In some of the above embodiments, R₉-R₁₇ are independently alkyl, alkenyl, alkynyl, aryl, substituted aryl, cycloalkyl, cycloheteroalkyl or heteroaryl. In other of the above embodiments, R₉-R₁₇ are independently alkyl, alkenyl, aryl, substituted aryl or cycloheteroalkyl. In still other of the above embodiments, R₉-R₁₇ are independently (C₁-C₄) alkyl, (C₁-C₄) alkenyl, phenyl, substituted phenyl or (C₅-C) cycloheteroalkyl.

In some of the above embodiments, the anomeric carbon is the S stereoisomer.

Coumarin derivatives of galactose and fucose derivatives include those illustrated in Table 1, below.

TABLE 1

| | |
|---|---|
| 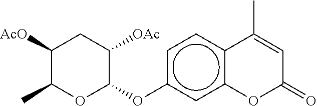 | 1 |
| 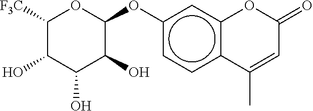 | 2 |
| 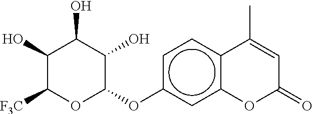 | 3 |
| 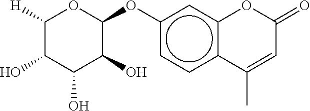 | 4 |
| 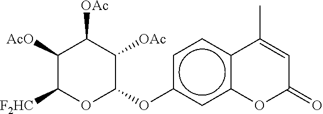 | 5 |
| 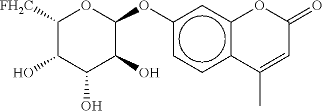 | 6 |
| 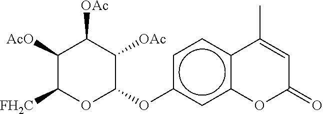 | 7 |
| 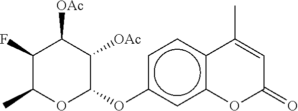 | 8 |
| 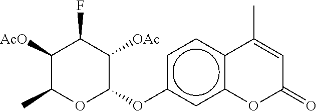 | 9 |
| 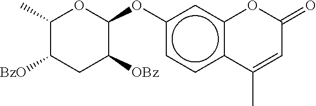 | 10 |
| 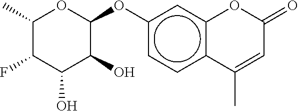 | 11 |
| 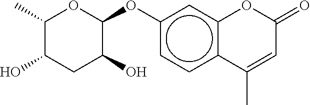 | 12 |
| 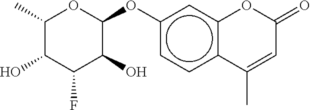 | 13 |
| 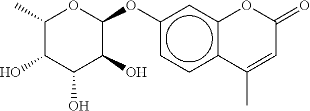 | 14 |
| 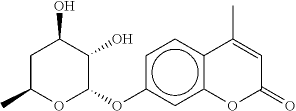 | 15 |
| 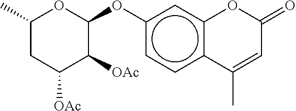 | 16 |
| 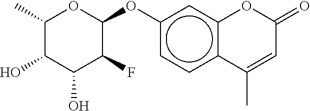 | 17 |
| 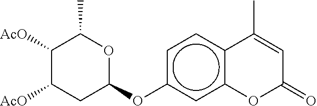 | 18 |
| 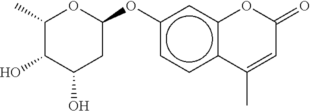 | 19 |
| 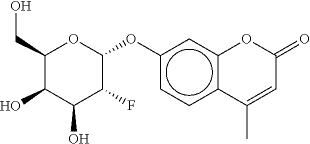 | 20 |
| 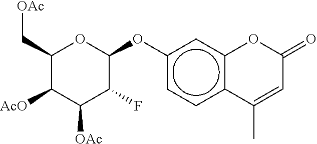 | 21 |
| 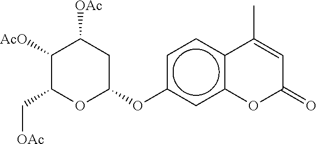 | 22 |
| 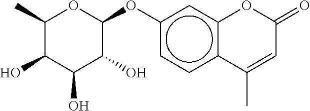 | 23 |
| 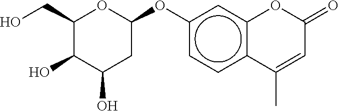 | 24 |
| 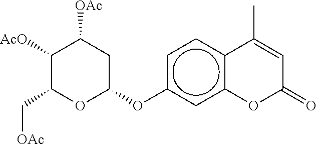 | 25 |
| 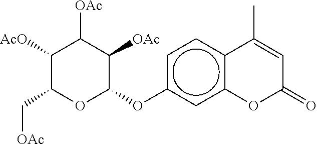 | 26 |
| 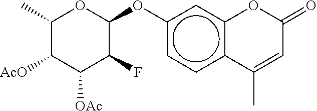 | 27 |
| 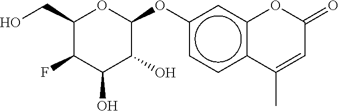 | 28 |
| 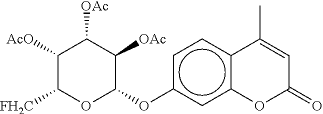 | 29 |
| 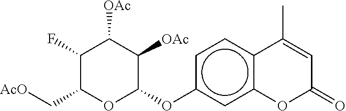 | 30 |
| 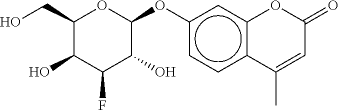 | 31 |
| 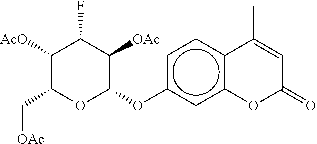 | 32 |
| 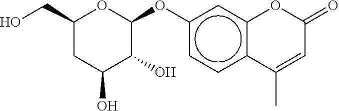 | 33 |
| 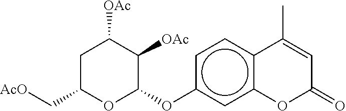 | 34 |
| 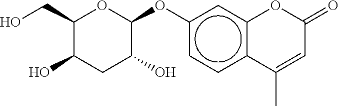 | 35 |
| 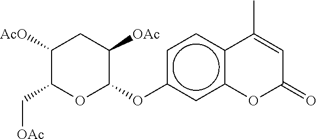 | 36 |
| 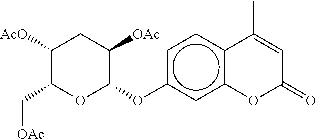 | 37 |
| 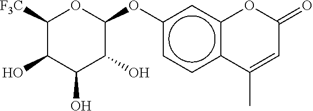 | 38 |
| 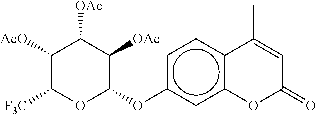 | 39 |
| 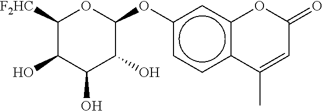 | 40 |
| 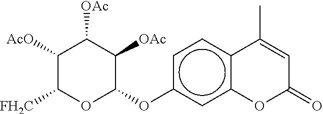 | 41 |
| 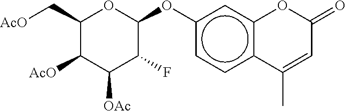 | 42 |
| 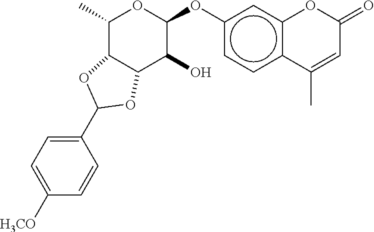 | 43 |
| 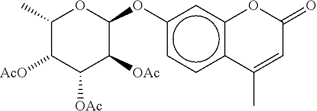 | 44 |
| 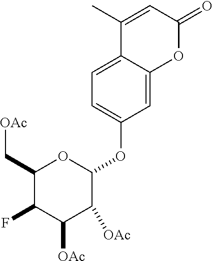 | 45 |
| 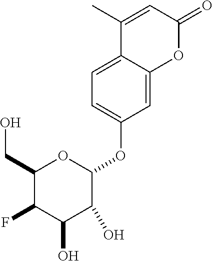 | 46 |
| 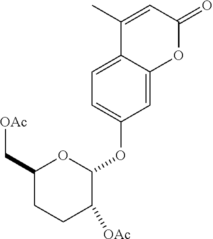 | 47 |
| 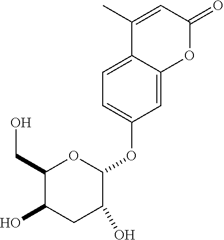 | 48 |
| 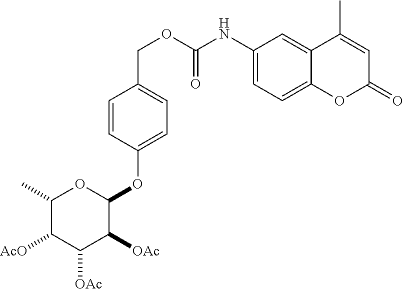 | 49 |
| 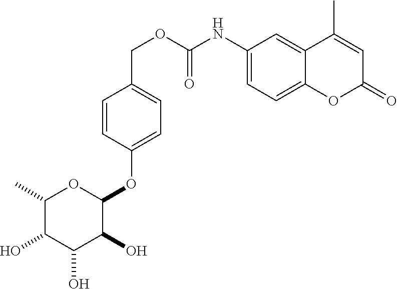 | 50 |
| 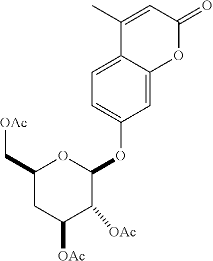 | 51 |
| 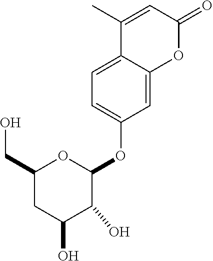 | 52 |
| 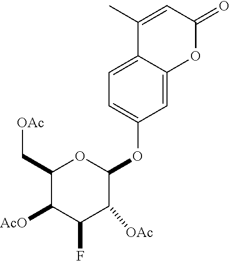 | 53 |
| 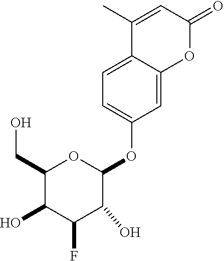 | 54 |
| 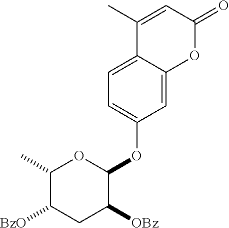 | 55 |
| 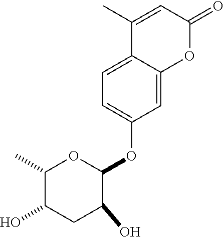 | 56 |
| 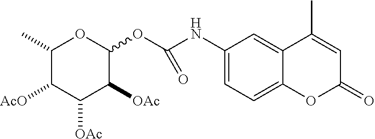 [SMILES: CC(=O)O[C@@H]1[C@H](OC(C)=O)[C@H](C)OC(OC(=O)Nc2ccc3oc(=O)cc(C)c3c2)[C@H]1OC(C)=O] | 57 |
| 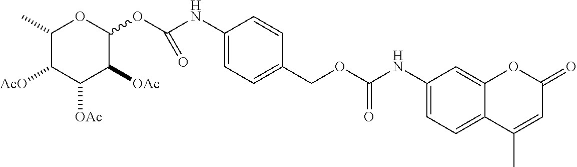 [SMILES: CC(=O)O[C@@H]1[C@H](OC(C)=O)[C@H](C)OC(OC(=O)Nc2ccc(COC(=O)Nc3ccc4c(C)cc(=O)oc4c3)cc2)[C@H]1OC(C)=O] | 58 |
| 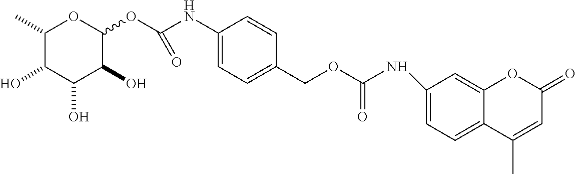 [SMILES: Cc1cc(=O)oc2cc(NC(=O)OCc3ccc(NC(=O)OC4O[C@@H](C)[C@@H](O)[C@@H](O)[C@@H]4O)cc3)ccc12] | 59 |

### DIAGNOSTIC COMPOSITIONS

The compositions provided herein contain diagnostically effective amounts of a compound provided herein and a vehicle. Vehicles suitable for diagnostically measuring the amount of a hydrolysis of compound provided herein include any such carriers known to those skilled in the art to be suitable for the particular diagnostic measurement.

The compounds are, in some embodiments, formulated into suitable preparations such as solutions, suspensions, powders, sustained release formulations or elixirs. In some embodiments, the compounds described above are formulated into compositions using techniques and procedures well known in the art.

In the compositions, effective concentrations of a compound are mixed with a suitable vehicle. The concentrations of the compound in the compositions is effective for a diagnostic measurement described herein. To formulate a composition, the weight fraction of a compound is dissolved, suspended, dispersed or otherwise mixed in a selected vehicle at an effective concentration such that a diagnostic measurement can be made.

The concentration of compound in the composition will depend on the physicochemical characteristics of the compound as well as other factors known to those of skill in the art. In instances in which the compounds exhibit insufficient solubility, known methods for solubilizing compounds may be used. Such methods are known to those of skill in this art, and include, but are not limited to, using co-solvents, such as dimethylsulfoxide (DMSO), using surfactants or surface modifiers, such as TWEEN®, complexing agents such as cyclodextrin or dissolution by enhanced ionization (i.e., dissolving in aqueous sodium bicarbonate).

Liquid compositions can, for example, be prepared by dissolving, dispersing, or otherwise mixing a compound and optional adjuvants in a vehicle, such as, for example, water, saline, aqueous dextrose, glycerol, glycols, ethanol, and the like, to thereby form a solution or suspension, colloidal dispersion, emulsion or liposomal formulation. If desired, the composition may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents. Other formulations include, but are not limited to, aqueous alcoholic solutions including an acetal. Alcohols used in these formulations are any water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal. The contemplated compositions may contain 0.001%-100% active ingredient, in one embodiment 0.1-95%, in another embodiment 0.4-10%.

The following examples are provided for illustrative purposes only and are not intended to limit the scope of the invention.

### EXAMPLES

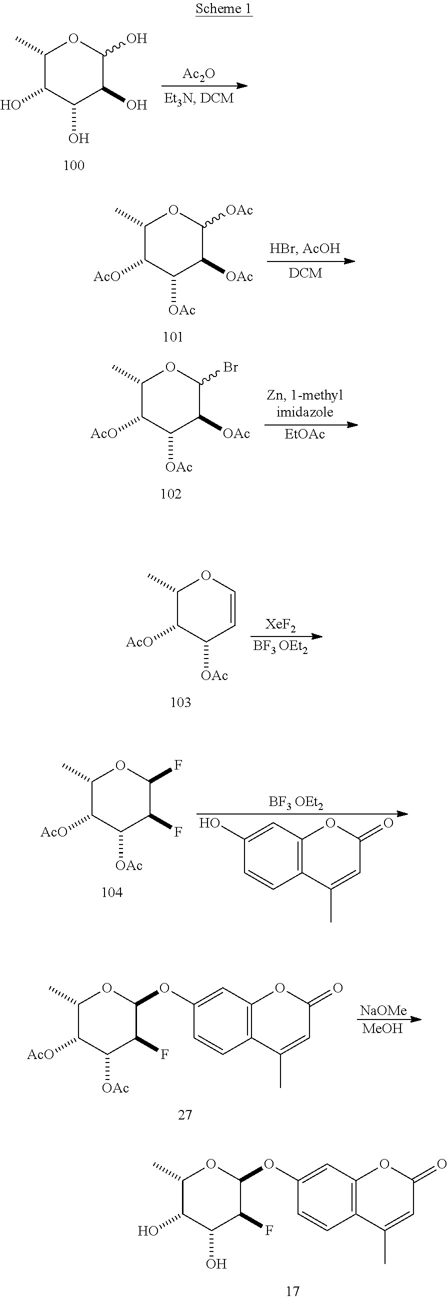

Scheme 1 illustrates preparation of compounds 17 and 27.

(3S,4R,5R,6S)-6-methyltetrahydro-2H-pyran-2,3,4,5-tetrayl tetraacetate (101)

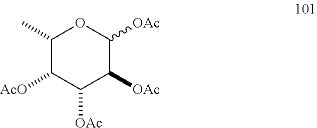 [SMILES: CC(=O)OC1O[C@@H](C)[C@@H](OC(C)=O)[C@@H](OC(C)=O)[C@@H]1OC(C)=O]

L-Fucose (3.0 g, 18.2 mmol, 1.0 equiv.) (100) was suspended in dichloromethane (50 mL). Triethyl amine (11 mL, 78.6 mmol, 4.3 equiv.) and acetic anhydride (7.26 mmol, 71.1 mmol, 3.9 eq) were added at room temperature under nitrogen atmosphere. The suspended mixture was cooled to ice bath and DMAP (145 mg, 1.12 mmol, 0.1 eq) was added in one portion. The reaction mixture was stirred under cooling temperature for at least 10 minutes. The ice bath was removed and stirring was continued until mixture was completely dissolved (2 h). The reaction was monitored by TLC and LC-MS to confirm the consumption of the starting material and formation of the product. The reaction mixture was washed with cold sat. NaHCO₃ (2×) and extracted with dichloromethane. The combined organic layers were washed with brine and dried over sodium sulfate. Volatiles were removed under reduced pressure to afford crude product as brown color oil. The oil product was treated with ethyl acetate/hexane and a solid was precipitated out of solution. The solid product (3S,4R,5R,6S)-6-methyltetrahydro-2H-pyran-2,3,4,5-tetrayl tetraacetate (101) was filtered out to as light pink solid. (5.8 g, 95% yield).
(2S,3S,4R,5R,6S)-2-bromo-6-methyltetrahydro-2H-pyran-3,4,5-triyl triacetate (102)

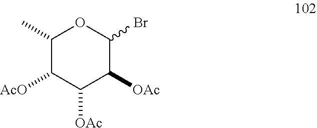 [SMILES: CC(=O)O[C@@H]1[C@H](OC(C)=O)[C@H](C)OC(Br)[C@H]1OC(C)=O]

(3S,4R,5R,6S)-6-methyltetrahydro-2H-pyran-2,3,4,5-tetrayl tetraacetate (3.4 g, 10.23 mmol) (101) was dissolved in dry dichloromethane (20 mL) and cooled to 0° C. HBr (33% in AcOH, 5 mL) was added and the reaction mixture was allowed to warm to room temperature and stirred for 3 h. The reaction mixture was poured onto an ice/water mixture and layers were separated. The aqueous phase was extracted twice with dichloromethane and the combined organic layers were washed with sat. NaHCO₃ and brine, then dried over Na₂SO₄. The solvent was removed under reduced pressure to yield the title compound (102) (3.25 g, 91%) as a yellow oil, which was used in the next step without further purification. Calculated mass: 352.02, Mass (ESI+) observed: 369.6 [M+(H₂O)]⁺.
(2S,3R,4S)-2-methyl-3,4-dihydro-2H-pyran-3,4-diyl diacetate (103)

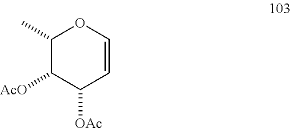

To a stirring solution of Zn (3.2 g, 54.0 mmol, 6.0 eq) and 1-methyl imidazole (1.2 mL, 9.9 mmol, 1.1 eq) in ethyl acetate (anhydrous) (65 mL) at boiling temperature (80° C.) was added (2S,3S,4R,5R,6S)-2-bromo-6-methyltetrahydro-2H-pyran-3,4,5-triyl triacetate (102) (3.2 g, 9.06 mmol, 1.0 eq) in ethyl acetate (7.0 mL) dropwise. The reaction mixture was stirred for 75 min at reflux with the consumption of starting material and formation of product monitored by TLC. The reaction mixture was stirred for another 30 min at room temperature, then filtered over a pad of celite which was washed with ethyl acetate 3× (100 mL). The combined organic layers were washed with 5% HCl (1×), NaHCO₃ (1×), brine and dried over sodium sulfate. Volatiles were removed under reduced pressure to afford a colorless oil. The crude product was purified by combi-flash chromatography using 25% ethyl acetate in hexanes. The product fractions were collected and concentrated to give pure product (103) as white solid (810 mg, 41% yield). ¹H NMR (500 MHz, Chloroform-d) δ 6.47 (dd, J=6.3, 2.0 Hz, 1H), 5.64−5.52 (m, 1H), 5.29 (dt, J=4.6, 1.6, 1.6 Hz, 1H), 4.65 (dt, J=6.3, 2.0, 2.0 Hz, 1H), 4.22 (q, J=6.6, 6.5, 6.5 Hz, 1H), 2.16 (s, 3H), 2.02 (s, 3H), 1.28 (d, J=6.7 Hz, 3H).

(2S,3R,4R,5S)-5,6-difluoro-2-methyltetrahydro-2H-pyran-3,4-diyl diacetate (104)

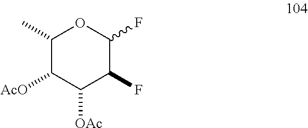 [SMILES: CC(=O)O[C@@H]1[C@H](OC(C)=O)[C@H](C)OC(F)[C@H]1F]

To a round bottom flask equipped with a nitrogen inlet and a magnetic stir bar was added a solution of (2S,3R,4S)-2-methyl-3,4-dihydro-2H-pyran-3,4-diyl diacetate (103) (410 mg, 1.91 mmol) in dry diethyl ether (13 mL) followed by XeF₂ (400 mg, 2.36 mmol). To the stirred suspension, a solution of BF₃-etherate in dry benzene (13 mL) was added dropwise over 10-15 mins and the reaction was stirred overnight at room temperature. The reaction mixture was cooled to 0° C. before a saturated aqueous solution of NaHCO₃ was added and the aqueous layer was extracted with ethyl acetate (3×). The combined organic layers were dried over anhydrous Na₂SO₄, iltered and volatiles were removed under reduced pressure to give (2S,3R,4R,5S)-5,6-difluoro-2-methyltetrahydro-2H-pyran-3,4-diyl diacetate (104) (448 mg, 93%) as white solid. ¹H NMR (500 MHz, Chloroform-d3): δ 5.81 (dd, J=53.4, 2.6 Hz, 1H), 5.42 (dddd, J=16.4, 8.7, 4.0, 2.1 Hz, 2H), 4.87−4.66 (m, 1H), 4.37 (q, J=6.5 Hz, 1H), 2.17 (s, 3H), 2.07 (s, 3H), 1.20 (d, J=6.5 Hz, 3H).

### Example 1: (2S,3R,4R,5S,6S)-5-fluoro-2-methyl-6((4-methyl-2-oxo-2H-chromen-7-yl)oxy)tetrahydro-2H-pyran-3,4-diyl diacetate(27)

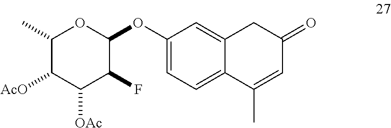

(2S,3R,4R,5S)-5,6-difluoro-2-methyltetrahydro-2H-pyran-3,4-diyl diacetate (104) (140 mg, 0.55 mmol, 1.0 eq) was dissolved in DCM (3.5 mL) and 7-hydroxy-4-methyl-2H-chromen-2-one (97 mg, 0.55 mmol, 1.0 eq) was added followed by triethyl amine (0.087 mL, 0.605 mmol, 1.1 eq). The mixture was cooled in an ice bath and BF₃ Et₂O was added (0.07 mL, 0.55 mmol, 1.0 eq). The ice bath was removed and the reaction was stirred at room temperature for 3 h. Another portion of BF₃ Et₂O (0.5 equiv.) was added and stirred for another 3 h, The reaction progress was monitored by LC-MS. The reaction was cooled to ice bath temperature and quenched with methanol (0.8 mL). The volatiles were removed under reduced pressure and the mixture was diluted with ethyl acetate. The solution was washed with 10% citric acid, water, NaHCO₃ solution and brine. The combined organic layers were concentrated to give the crude product as brown solid. The crude product was subjected to normal phase purification (10 g silica gel column) eluting with 10% to 50% ethyl acetate in hexanes gradient. Product fractions were collected and concentrated to give the product (27) as white foamy solid (28 mg). Calculated mass: 408.12, Mass (ESI+) observed: 409.5 [M+H]⁺.

### Example 2: (2S,3R,4R,5S)-5,6-difluoro-2-methyltetrahydro-2H-pyran-3,4-diyl dihydroxy(17)

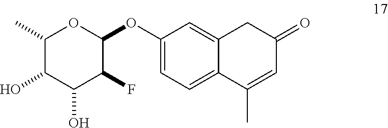

To a solution of (2S,3R,4R,5S,6S)-5-fluoro-2-methyl-644-methyl-2-oxo-2 H-chromen-7-yl)oxy)tetrahydro-2H-pyran-3,4-diyl diacetate (27) (28 mg, 0.068 mmol, 1.0 eq) in methanol (0.8 mL) was added 25% NaOMe in methanol (0.015 mL, 0.074 mmol, 1.0 eq) at ice bath temperature. The reaction mixture was stirred for 15 minutes and monitored by LC-MS. 1N HCl/dioxane was added until pH 7 then concentrated to afford crude product (19) which was purified by reverse phase HPLC. ¹H NMR (500 MHz, DMSO-d6) δ 7.74 (dd, J=9.8, 4.1 Hz, 1H), 7.20−7.05 (m, 2H), 6.29−6.17 (m, 1H), 5.85 (d, J=4.0 Hz, 1H), 4.21 (ddd, J=11.7, 9.9, 3.5 Hz, 1H), 4.13−3.99 (m, 1H), 3.88−3.75 (m, 1H), 2.54−2.41 (m, 3H), 1.21 (t, J=6.0, 6.0 Hz, 3H). Calculated mass: 324.1, Mass (ESI⁺) observed: 325.3 [M⁺H]⁺.

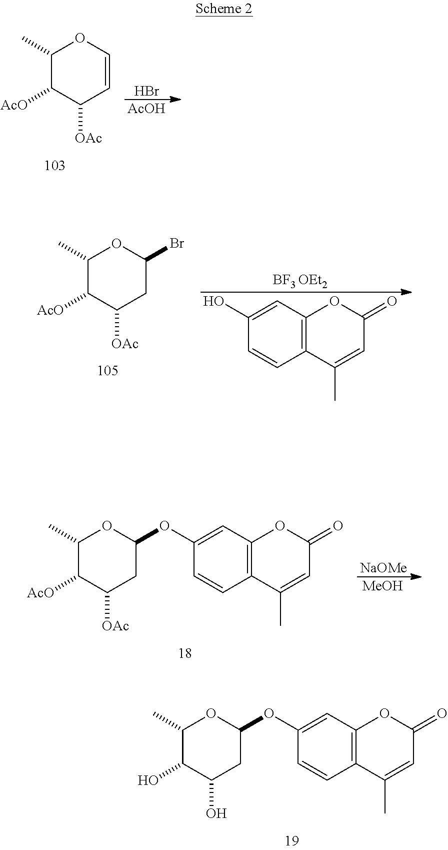

Scheme 2 illustrates the preparation of compounds 18 and 19.

(2S,3R,4S,6S)-6-bromo-2-methyltetrahydro-2H-pyran-3,4-diyl diacetate (105)

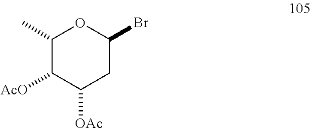 [SMILES: CC(=O)O[C@@H]1[C@H](C)O[C@@H](Br)C[C@@H]1OC(C)=O]

(2S,3R,4S)-2-methyl-3,4-dihydro-2H-pyran-3,4-diyl diacetate (103) (75 mg, 0.35 mmol, 1.0 eq) was dissolved in dichloromethane (0.5 mL). A solution of HBr (0.15 mL, 1.4 mmol, 4.0 eq, 33% in acetic acid) in dichloromethane (0.3 mL) was added dropwise to the reaction mixture at -10° C. (ice bath/salt). The reaction became a dark yellow in 15 min at which time TLC indicated the consumption of the starting material. The reaction was quenched with ice water and diluted with ethyl acetate. The organic layer was washed with ice water, saturated aqueous NaHCO₃, brine and dried over sodium sulfate. Volatiles were removed under reduced pressure to afford the crude product (2S,3R,4S,6S)-6-bromo-2-methyltetrahydro-2H-pyran-3,4-diyl diacetate (105) as a colorless oil (70 mg, 67% yield), which was used in the next step without further purification. Example 3: (2S,3R,4S,6S)-2-methyl-6-((4-methyl-2-oxo-2H-chromen-7-yl)oxy)tetrahydro-2H-pyran-3,4-diyl diacetate (18)

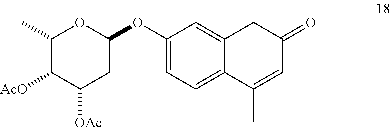

To a solution of (2S,3R,4S,6S)-6-bromo-2-methyltetrahydro-2H-pyran-3,4-diyl diacetate (90 mg, 0.3 mmol, 1.0 equiv.) (105) in DCM (2.5 mL) was added 7-hydroxy-4-methyl-2H-chromen-2-one (53 mg, 0.3 mmol, 1.0 equiv.). BF₃ Et₂O (0.041 mL, 0.33 mmol, 1.0 equiv.) was added dropwise to the reaction mixture at ice bath temperature. The reaction mixture turned dark yellow in 15 min and the reaction was monitored by TLC which showed the consumption of the starting material. The reaction was quenched with ice water and diluted with ethyl acetate. The organic layer was washed with ice water, saturated aqueous NaHCO₃, brine and dried over sodium sulfate. Volatiles were removed under reduced pressure to afford the crude product (75 mg). The reaction was repeated on 2× scale, the crude products were combined and purified by normal phase silica gel using 5-40% ethyl acetate in hexanes. Product fractions were collected and concentrated to give the product (18) as a white solid (35 mg). ¹H NMR (500 MHz, DMSO-d6) δ 7.52 (d, J=8.7 Hz, 1H), 7.09 (d, J=2.4 Hz, 1H), 6.99 (dd, J=8.7, 2.4 Hz, 1H), 6.23−6.13 (m, 1H), 5.79 (d, J=3.4 Hz, 1H), 5.49 (ddd, J=12.3, 5.1, 3.0 Hz, 1H), 5.25 (d, J=3.0 Hz, 1H), 4.11 (q, J=6.5, 6.5, 6.5 Hz, 1H), 2.44−2.39 (m, 3H), 2.33−2.23 (m, 1H), 2.20 (d, J=0.9 Hz, 3H), 2.19−2.07 (m, 1H), 2.04 (d, J=0.8 Hz, 3H), 1.11 (d, J=6.4 Hz, 3H). Calculated mass: 390.13, Mass (ESI+) observed: 391.3 [M⁺H]⁺.

### Example 4: (2S,3R,4S,6S)-2-methyl-6-((4-methyl-2-oxo-2H-chromen-7-yl)oxy)tetrahydro-2H-pyran-3,4-diyl diol (19)

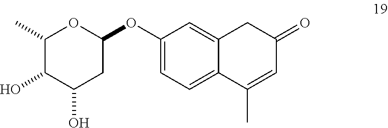

(2S,3R,4S,6S)-2-methyl-6-((4-methyl-2-oxo-2H-chromen-7-yl)oxy)tetrahydro-2H-pyran-3,4-diyl diacetate (18) (50 mg, 0.12 mmol, 1.0 eq) was suspended in methanol (1.2 mL) and 25% NaOMe in methanol (0.027 mL, 0.128 mmol, 1.0 eq) was added at ice bath temperature. The reaction mixture suspension dissolved within 10 min and reaction was monitored by LC-MS. 1N HCl was added until the pH was 6. The mixture was concentrated and the product was triturated with ether and hexanes (3×). The resulting solid was purified by reverse phase HPLC to give 40 mg of (2S,3R,4S,6S)-2-methyl-6((4-methyl-2-oxo-2H-chromen-7-yl)oxy)tetrahydro-2H-pyran-3,4-diyl diol (19). ¹H NMR (500 MHz, DMSO-d6) δ 7.75 −7.63 (m, 1H), 7.09−6.96 (m, 2H), 6.29−6.16 (m, 1H), 5.81 (d, J=3.3 Hz, 1H), 4.73 (dd, J=6.2, 1.0 Hz, 1H), 4.52 (d, J=4.7 Hz, 1H), 4.03−3.88 (m, 1H), 3.75 (q, J=6.5, 6.5, 6.5 Hz, 1H), 3.45 (t, J=3.9, 3.9 Hz, 1H), 2.38 (d, J=1.2 Hz, 3H), 1.97 (td, J=12.6, 12.6, 3.6 Hz, 1H), 1.77 (dd, J=13.0, 4.9 Hz, 1H), 1.03 (dd, J=6.5, 1.0 Hz, 3H). Calculated mass: 306.11, Mass (ESI+) observed: 307.0 [M⁺H]⁺.

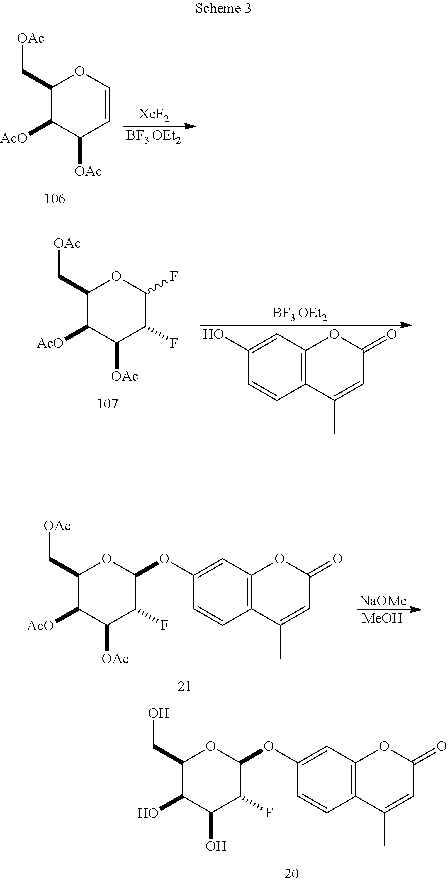

Scheme 3 illustrates the synthesis of compounds 20 and 21.

(2R,3S,4S,5R)-2-(acetoxymethyl)-5,6-difluorotetrahydro-2H-pyran-3,4-diyl diacetate (107)

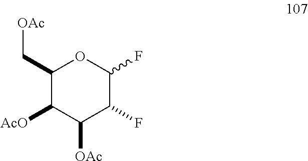 [SMILES: CC(=O)OC[C@H]1OC(F)[C@H](F)[C@@H](OC(C)=O)[C@H]1OC(C)=O]

To a mixture of tri-O-acetyl-D-galactal (106) (870 mg, 3.2 mmol) and XeF₂ (540 mg, 3.2 mmol) in anhydrous Et₂O (17 mL) under N2 was added dropwise a solution of BF₃. Et₂ (0.4 mL, 1 mmol) in anhydrous benzene (16 mL). The resulting mixture was stirred at room temperature for 2 h then washed with a saturated NaHCO₃ solution (2×), water and dried over MgSO₄. Volatiles were removed under reduced pressure to afford the crude product which was purified by normal silica gel using 20-30% ethyl acetate in hexanes. The product fractions were collected and concentrated to give (2R,3S,4S,5R)-2-(acetoxymethyl)-5,6-difluorotetrahydro-2H-pyran-3,4-diyl diacetate (107) as a white solid (961 mg, 97% yield). ¹H NMR (500 MHz, DMSO-d6) δ 5.86 (dd, J=53.1, 2.9 Hz, 1H), 5.61−5.51 (m, 1H), 5.42 (td, J=10.9, 10.7, 3.5 Hz, 1H), 4.90−4.68 (m, 1H), 4.47−4.38 (m, 1H), 4.13 (dd, J=6.5, 2.3 Hz, 2H), 2.16 (s, 3H), 2.07 (s, 6H).

### Example 5: (2R,3S,4S,5R,6S)-2-(acetoxymethyl)-5-fluoro-6-((4-methyl-2-oxo-2H-chromen-7-yl)oxy)tetrahydro-2H-pyran-3,4-diyl diacetate (21)

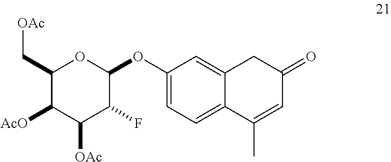

Activated 4 Å molecular sieves (500 mg) and 7-hydroxy-4-methyl-2H-chromen-2-one (119 mg, 0.674 mmol) were added to a solution of (2R,3S,4S,5R)-2-(acetoxymethyl)-5,6-difluorotetrahydro-2H-pyran-3,4-diyl diacetate (107) (190 mg, 0.612 mmol) in acetonitrile (6.0 ml). The resulting suspension was stirred under nitrogen atmosphere for 30 min, then BF₃. OEt₂ (0.19 mL, 1.53 mmol) was added and the suspension was stirred in the absence of light under nitrogen for 12 h. The reaction was analyzed by LC-MS and TLC which indicated a mixture of two anomers (α:β, ˜2:1). The reaction was passed through celite, concentrated under reduced pressure and purified by reverse phase column chromatography to give (2R,3S,4S,5R,6S)-2-(acetoxymethyl)-5-fluoro-6-((4-methyl-2-oxo-2H-chromen-7-yl)oxy)tetrahydro-2H-pyran-3,4-diyl diacetate (21) (30 mg). ¹H NMR (500 MHz, DMSO-d6) δ 7.77 (d, J=8.5 Hz, 1H), 7.17 (q, J=2.4, 2.3, 2.3 Hz, 1H), 7.15 (d, J=2.4 Hz, 1H), 6.28 (q, J=1.3, 1.3, 1.3 Hz, 1H), 6.23 (d, J=3.9 Hz, 1H), 5.50 (ddd, J=11.3, 10.1, 3.6 Hz, 1H), 5.45 (td, J=3.5, 3.5, 1.3 Hz, 1H), 5.02 (ddd, J=48.5, 10.2, 3.8 Hz, 1H), 4.38−4.32 (m, 1H), 4.09−3.89 (m, 2H), 2.41 (d, J=1.3 Hz, 3H), 2.14 (s, 3H), 2.02 (s, 3H), 1.83 (s, 3H). Calculated mass: 466.12, Mass (ESI+) observed: 467.2 [M+H]⁺.

### Example 6: (2R,3S,4S,5R,6S)-2-(acetoxymethyl)-5-fluoro-6-((4-methyl-2-oxo-2H-chromen-7-yl)oxy)tetrahydro-2H-pyran-3,4-diyl diol (20)

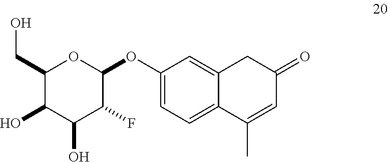

(2R,3S,4S,5R,6S)-2-(acetoxymethyl)-5-fluoro-6-((4-methyl-2-oxo-2H-chromenyl)oxy)tetrahydro-2H-pyran-3,4-diyl diacetate (21) (19 mg) was suspended in MeOH:H₂O (2:1, 1.5 mL), and triethyl amine (0.2 mL) was added. The reaction mixture was heated to 40° C. and stirred for 2 h. The volatiles were removed under vacuum and the crude product was purified by reverse phase HPLC using NH₄CO₃ as a modifier to afford the pure product (7.2 mg). ¹H NMR (500 MHz, DMSO-d6) δ 7.71 (d, J=8.7 Hz, 1H), 7.09 (d, J=2.4 Hz, 1H), 7.06 (dd, J=8.7, 2.5 Hz, 1H), 6.25 (q, J=1.2, 1.2, 1.2 Hz, 1H), 5.42 (dd, J=7.6, 3.8 Hz, 2H), 4.89 (d, J=3.9 Hz, 1H), 4.74 (t, J=5.5, 5.5 Hz, 1H), 4.57−4.39 (m, 1H), 3.77 (d, J=6.1 Hz, 3H), 3.52 (dq, J=17.0, 4.9, 4.9, 4.5 Hz, 2H), 2.40 (d, J=1.3 Hz, 3H). Calculated mass: 340.09, Mass (ESI+) observed: 341.2 1 [M⁺H]⁺.

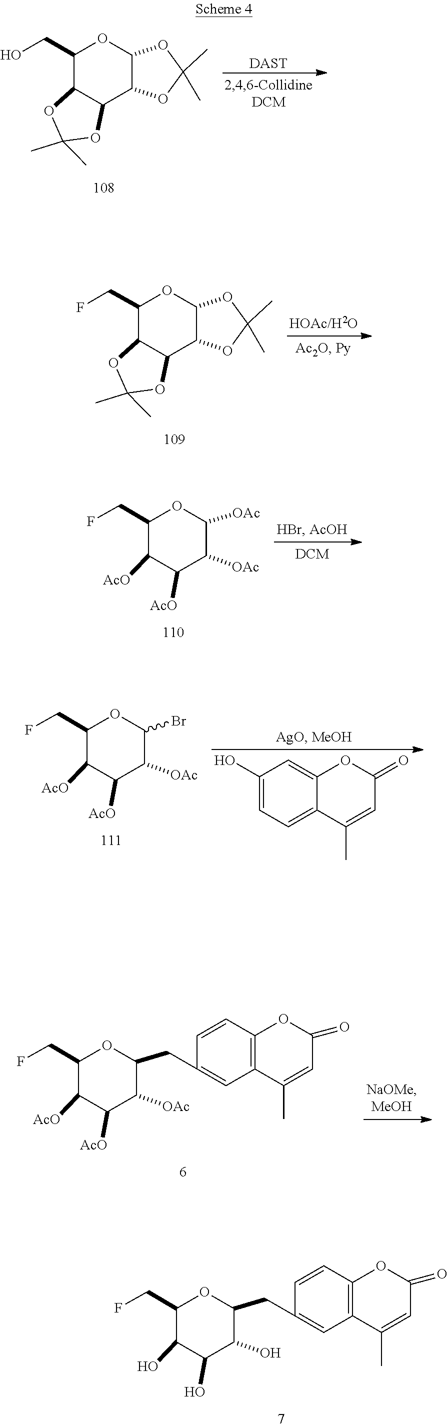

Scheme 4 illustrates the synthesis of compounds 6 and 7.

6-fluoro diacetone-D-galactose (109)

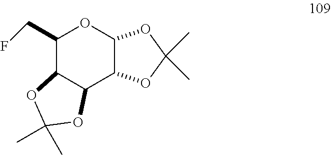

To a solution of commercially available diacetone-D-galactose (108) (1.0 g, 3.84 mmol) in anhydrous dichloromethane (6 mL) was added 2,4,6-collidine (1.12 g, 4.61 mmol) and DAST (743 mg, 4.61 mmol). The mixture was irradiated in microwave reactor at 80° C. for 1 h. The reaction mixture was cooled to room temperature, quenched with H₂O and extracted into dichloromethane (2×). The combined organic extracts were successively washed with saturated NaHCO₃ and brine. The organic phase was dried over Na₂SO₄, filtered, and concentrated under reduced pressure. The crude was purified by normal phase silica gel (0-30% EtOAc in hexanes) to afford the product (109) as a pale-yellow oil (620 mg, 62%). ¹H NMR (CDCl3, 500 MHz): 5.55 (d, J=5.7 Hz, 1H); 4.66-4.55 (m, 2H), 4.55−4.43 (m,1H), 4.34 (tt, J=4.8, 4.8, 2.4, 2.4 Hz, 1H), 4.27 (dt, J=7.3, 3.7, 3.7 Hz, 1H), 4.08 (dddd, J=12.2, 7.1, 5.1, 2.1 Hz, 1H) 1.55 (m, 3H), 1.45 (m, 3H), and 1.34 (s, 6H).

(2R,3R,4S,5R,6S)-6-(fluoromethyptetrahydro-2H-pyran-2,3,4,5-tetrayl tetraacetate (110)

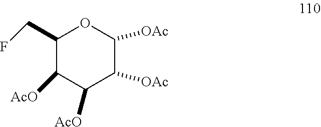 [SMILES: CC(=O)O[C@H]1O[C@H](CF)[C@H](OC(C)=O)[C@H](OC(C)=O)[C@H]1OC(C)=O]

The fluorinated compound (109) (620 mg) was dissolved in AcOH (10 mL, 80% aqueous solution) and refluxed for 20 h at 110° C. Volatiles were removed under reduced pressure, and the residue was co-evaporated with toluene (3×10 mL). The crude residue was dried under vacuo for 2 h and dissolved in pyridine (10 mL) and Ac₂O (5 mL). The reaction mixture was stirred at room temperature for 18 h then the solvent was removed under reduced pressure. The crude product was purified by normal phase silica gel (0-70% EtOAc in hexanes) and the pure product (110) was obtained as pale yellow oil (700 mg, 67%). Calculated mass: 350.10, Mass (ESI+) observed: 369.1 [(M+H)+H₂O]⁺

### Example 7: Preparation of Compound 6

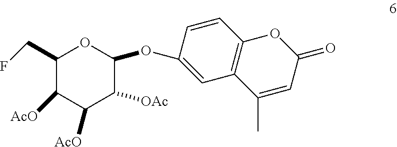 [SMILES: CC(=O)O[C@H]1[C@@H](OC(C)=O)[C@@H](CF)O[C@@H](Oc2ccc3oc(=O)cc(C)c3c2)[C@@H]1OC(C)=O]

(2R,3R,4S,5R,6S)-6-(fluoromethyl)tetrahydro-2H-pyran-2,3,4,5-tetrayl tetraacetate (350 mg, 1 mmol) (110) was dissolved in anhydrous CH₂Cl₂ (5 mL) and cooled to 0° C. HBr (33% in AcOH, 0.3 mL) was added. The reaction mixture was allowed to warm to room temp and stirred for 2 h. The reaction mixture was then poured into an ice/water mixture and separated. The aqueous phase was extracted with dichloromethane. The combined organic layers were washed with saturated NaHCO₃, brine and dried over Na₂SO₄. After filtration, the solvent was removed under reduced pressure to yield the crude product as a yellow oil, which was used in the next step without further purification. The crude residue was dissolved in anhydrous acetonitrile (10 mL) and 7-hydroxy-4-methylcoumarin (176 mg, 1 mmol) was added followed by Ag₂O (232 mg, 1 mmol). The reaction mixture was stirred at room temperature for 16 h. The reaction mixture was filtered and the filtrate was concentrated to afford the crude product which was purified by reverse phase HPLC (NH₄HCO₃ as modifier) to afford compound 6 as white solid (76 mg). ¹H NMR (500 MHz, DMSO-d6) δ 7.73 (d, J=8.9 Hz, 1H), 7.04 (d, J=2.4 Hz, 1H), 6.99 (dd, J=8.8, 2.4 Hz, 1H), 6.27 (d, J=1.3 Hz, 1H), 5.73−5.67 (m, 1H), 5.43−5.38 (m, 1H), 5.30−5.20 (m, 2H), 4.57 (dtd, J=18.5, 8.6, 8.3, 3.7 Hz, 2H), 4.52−4.47 (m, 1H), 4.42 (dd, J=9.8, 7.3 Hz, 1H), 2.40 (d, J=1.3 Hz, 3H), 2.12 (s, 3H), 2.02 (s, 3H), 1.94 (s, 3H). Calculated mass: 466.13, Mass (ESI+) observed: 467.2 [M⁺H]⁺.

### Example 8: Preparation of Compound 7

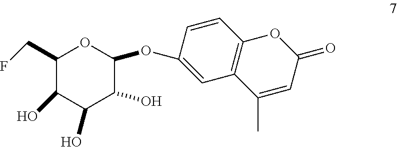 [SMILES: Cc1cc(=O)oc2ccc(O[C@@H]3O[C@H](CF)[C@H](O)[C@H](O)[C@H]3O)cc12]

To a solution of compound 6 (40 mg, 0.086 mmol) in MeOH (1.6 mL) and H₂O (0.4 mL) was added Et₃N (43 mg, 0.43 mmol) and the reaction mixture was stirred at 50° C. for 1 hour. After reaction was complete, the volatiles were removed under reduced pressure. The crude product was purified by reverse phase HPLC (NH₄HCO₃ as a modifier) to afford compound 7 as a white solid (24.5 mg). ¹H NMR (500 MHz, DMSO-d6) δ 7.73−7.68 (m, 1H), 7.02 (dd, J=6.8, 2.6 Hz, 2H), 6.24 (d, J=1.3 Hz, 1H), 5.30 (d, J=5.1 Hz, 1H), 5.08 (d, J=7.7 Hz, 1H), 5.02 (d, J=5.7 Hz, 1H), 4.79 (d, J=4.6 Hz, 1H), 4.64−4.37 (m, 2H), 4.07 (ddd, J=15.4, 7.7, 3.4 Hz, 1H), 3.75−3.69 (m, 1H), 3.61 (ddd, J=9.6, 7.7, 5.1 Hz, 1H), 3.46 (ddd, J=9.3, 5.7, 3.4 Hz, 1H), 2.39 (d, J=1.3 Hz, 3H). Calculated mass: 340.10, Mass (ESI+) observed: 340.9 [M+H]⁺.

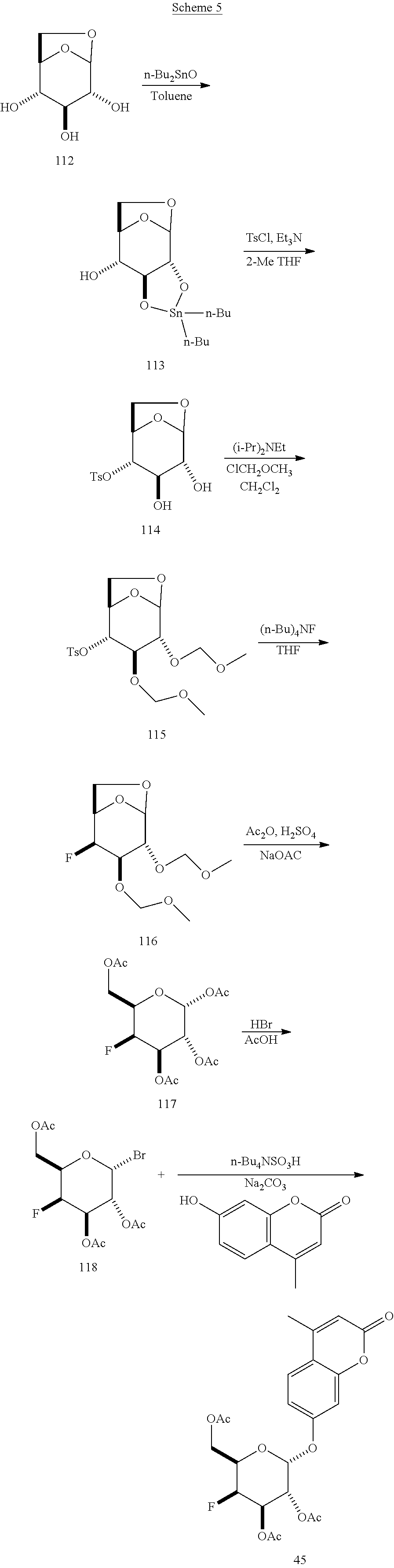

Scheme 5 illustrates the synthesis of compound 45.

Preparation of 1R,2R,6S,7R,8R)-4,4-dibutyl-3,5,10,11-tetraoxa-4-stannatricyclo[6.2.1.02,6]undecan-7-ol (113)

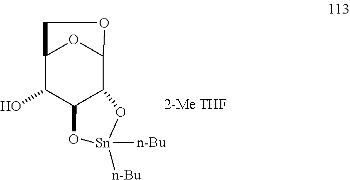

A mixture of 1,6-anhydro-β-D-glucose (112) (5.00 g, 30.8 mmol, 1.00 eq) and dibutyltin(IV) oxide (7.68 g, 30.8 mmol, 1.00 eq) in toluene (150 mL) was refluxed for 12 h in an apparatus equipped for the azeotropic removal of water (see Grindley et al., Carbohydrate Res. 1988, 172, 311). The cooled mixture was evaporated under reduced pressure to give the crude stannylene derivative (113) as a white semi-solid, which was used without purification.

Preparation of 1,6-anhydro-4-O-p-tolylsulfonyl-β-D-glucopyranose (114)

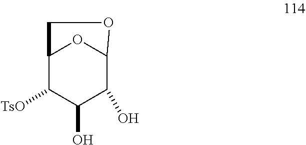 [SMILES: Cc1ccc(S(=O)(=O)O[C@H]2[C@H](O)[C@@H](O)C3OC[C@H]2O3)cc1]

To a solution of (1R,2R,6S,7R,8R)-4,4-dibutyl-3,5,10,11-tetraoxa-4-stannatricyclo[6.2.1.02,6]undecan-7-ol (113) (12.54 g, 31.9 mmol, 1.00 eq) in tetrahydrofuran (300 mL) was added triethylamine (4.9 mL, 35.1 mmol, 1.10 eq) and powdered 4A molecular sieves (3 g). p-Toluenesulfonyl chloride (6.69 g, 35.1 mmol, 1.10 eq) was added and the mixture was stirred vigorously for 2 days and then filtered through Celite. The filtrate was evaporated, and the residue was diluted with dichloromethane (150 mL). The organic solution was washed with water (2×50 mL), dried (sodium sulfate) and evaporated. The crude material was purified by column chromatography on silica gel using 7:3 dichloromethane: 2-methyltetrahydrofuran as the eluant. The first component to elute was 1,6-anhydro-2,4-di-O-p-tolylsulfonyl-β-D-glucopyranose which separated easily. The second component was the desired product (114) (˜8 g colorless oil) which was contaminated with the other regioisomer 1,6-anhydro-2-0-p-tolylsulfonyl-β-D-glucopyranose which was difficult to separate. The mixture was recrystallized from a mixture of acetone, ether, and petroleum ether (b.p. 30-60° C.) to give the desired product (114) as white needles. A second recrystallisation gave the pure product (2.2 g, 22%) as a single regioisomer. ¹H NMR (400 MHz, CDCl₃): d 7.83 (d, J=8.3 Hz, 2H), 7.38 (d, J=8.1 Hz, 2H), 5.48 (s, 1H), 4.65 (d, J=5.4 Hz, 1H), 4.42 (s, 1H), 4.13 (d, J=8.1 Hz, 1H), 3.79−3.71 (m, 2H), 3.49 (dd, J=0.9, 11.3 Hz, 1H), 2.50 (d, J=7.5 Hz, 1H), 2.47 (s, 3H), 2.32 (d, J=11.4 Hz, 1H).

Preparation of 1,6-Anhydro-2,3-bis(O-methoxymethyl)-4-O-(4-toluenesulfonyl)-β-D-glucopyranose (115)

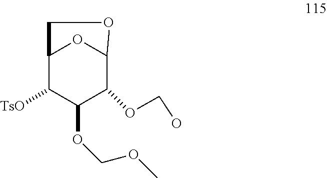 [SMILES: COCO[C@H]1[C@H](OS(=O)(=O)c2ccc(C)cc2)[C@H]2COC(O2)[C@@H]1OCO]

To a stirred solution of [(1R,2S,3R,4R,5R)-3,4-dihydroxy-6,8-dioxabicyclo[3.2.1]octan-2-yl] 4-methylbenzenesulfonate (114) (2.20 g, 6.95 mmol, 1.00 eq) in dichloromethane (50 mL) were added N,N-diisopropylethylamine (13 mL, 76.5 mmol, 11.0 eq) and chloromethyl methyl ether (5.3 mL, 69.5 mmol, 10.0 eq). The mixture was stirred at 40° C. for 4 h resulting in a brown solution. The solution was cooled and then quenched with water (50 mL). The mixture was extracted with dichloromethane (2×50 mL) and the combined organic phases were washed with brine (100 mL). The organic solution was dried over sodium sulfate, filtered, and concentrated under reduced pressure. The crude residue was purified by flash column chromatography (silica gel 40 g, ethyl acetate/hexanes, (5-50%)) to give [(1R,2R,3R,4R,5R)-3,4-bis(methoxymethoxy)-6,8-dioxabicyclo[3.2.1]octan-2-yl] 4-methylbenzenesulfonate (115) (2.10 g, 5.19 mmol, 75%) as a colorless oil). Rf =0.5 (silica, ethyl acetate/cyclohexane 1:1). ¹H NMR (400 MHz, CDC1₃): d 7.84 (d, J=8.3 Hz, 2H), 7.36 (d, J=8.1 Hz, 2H), 5.46 (s, 1H), 4.68−4.63 (m, 2H), 4.59 (s, 2H), 4.58 - 4.53 (m, 1H), 4.44 (s, 1H), 4.04 (d, J=7.7 Hz, 1H), 3.86−3.84 (m, 1H), 3.71 (dd, J=6.0, 7.5 Hz, 1H), 3.52 - 3.50 (m, 1H), 3.37 (s, 3H), 3.32 (s, 3H), 2.45 (s, 3H).

Preparation of 1,6-Anhydro-4-deoxy-4-fluoro-2,3-bis(O-methoxymethyl)-β-D-galactopyranose) (116)

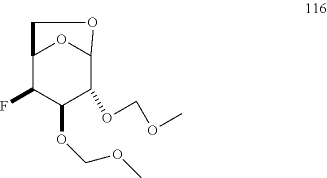 [SMILES: COCO[C@H]1[C@@H](F)[C@H]2COC(O2)[C@@H]1OCOC]

[(1R,2R,3R,4R,5R)-3,4-bis(methoxymethoxy)-6,8-dioxabicyclo[3.2.1]octan-2-yl] 4-methylbenzenesulfonate (115) (2.10 g, 5.19 mmol, 1.00 eq) was stirred with tetrabutylammonium fluoride (1M in THF, 55 mL, 10 equiv.) under reflux for 5 days. The black mixture was cooled and evaporated. The residue was diluted with water (100 mL) and the mixture was extracted with ethyl acetate (3×50 mL). The combined organic phases were washed with brine (100 mL), dried over sodium sulfate, filtered, and concentrated under reduced pressure. The crude material was purified by flash column chromatography (silica gel, ethyl acetate/cyclohexane, 0-30%) to give (1R,2S,3R,4R,5R)-2-fluoro-3,4-bis(methoxymethoxy)-6,8-dioxabicyclo [3.2.1]octane (116) as a pale-yellow oil (470 mg, 25% yield, 70% purity). This inseparable mixture containing the desired fluoro product and an unknown other product was used for the next step without further purification. Rf=0.51 (silica, ethyl acetate/cyclohexane 2:3). ¹H NMR (400 MHz, CDCl₃) was consistent with the product (116) as the major component (˜70% pure).
Preparation of 1,2,3,6-Tetra-O-acetyl-4-deoxy-4-fluoro-α/β-D-galactopyranose (117)

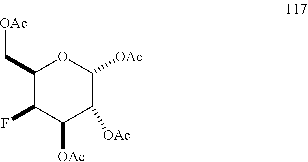 [SMILES: CC(=O)OC[C@H]1O[C@H](OC(C)=O)[C@H](OC(C)=O)[C@@H](OC(C)=O)[C@H]1F]

To a stirred solution of the mixture containing compound (1R,2S,3R,4R,5R)-2-fluoro-3,4-bis(methoxymethoxy)-6,8-dioxabicyclo[3.2.1]octane (116) (470 mg, 1.86 mmol, 1.00 eq) (70% pure) in acetic anhydride (5.3 mL, 55.9 mmol, 30.0 eq) at 0° C. was added sulfuric acid (0.99 mL, 18.6 mmol, 10.0 eq) dropwise. The mixture was stirred at room temperature for 72 h. The mixture was then cooled to 0° C., and sodium acetate (3.06 g, 37.3 mmol, 20.0 eq) was added, stirred for an additional 20 minutes and then quenched with water (20 mL). The mixture was extracted with dichloromethane (3×15 mL). The combined organic phases were successively washed with water (3×30 mL) and brine (30 mL), dried over sodium sulfate, filtered, and concentrated under reduced pressure. The crude residue was purified by flash column chromatography (silica gel, 12 g 15 μm, ethyl acetate in cyclohexane, 1-40%) to give an anomeric mixture (α/β=4:1) of product [(2R,3S,4R,5R)-4,5,6-triacetoxy-3-fluoro-tetrahydropyran-2-yl]methyl acetate (117) (360 mg, 0.925 mmol, 50%) as a colorless oil (360 mg, 90% pure, ˜50% yield). Rf 32 0.4 (silica, AcOEt/hexanes, 1:1). ¹H NMR (400 MHz, CDCl₃): δ 6.39 (d, J=3.5 Hz, 1H), 5.43−5.39 (m, 1H), 5.32−5.21 (m, 1H), 4.97 (dd, J=2.7, 50.2 Hz, 1H), 4.32−4.16 (m, 3H), 2.16 (s, 3H), 2.14 (s, 3H), 2.09 (s, 3H), 2.03 (s, 3H).

[(2R,3S,4R,5R)-4,5-diacetoxy-6-bromo-3-fluoro-tetrahydropyran-2-yl] methyl acetate (118)

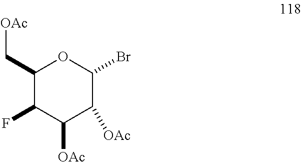 [SMILES: CC(=O)OC[C@H]1O[C@H](Br)[C@H](OC(C)=O)[C@@H](OC(C)=O)[C@H]1F]

To a stirred solution of [(2R,3S,4R,5R)-4,5,6-triacetoxy-3-fluoro-tetrahydropyran yl]methyl acetate (117) (360 mg, 1.03 mmol, 1.00 eq) in dichloromethane (6.00 mL) at 0° C., was added 6 M hydrogen bromide (4.0 mL, 24.0 mmol, 23.4 eq) as a 33 wt % solution in AcOH. The mixture was stirred at room temperature for 1 h and then quenched at 0° C. with a saturated aqueous NaHCO₃ solution (20 mL). The dichloromethane layer was passed through a hydrophobic frit and was not evaporated. TLC (50:50 ethyl acetate: cyclohexane) showed a less polar spot and most of the SM had reacted. The crude bromide (118) was used as a solution in dichloromethane for the next step without further purification.

### Example 9: [(2R,3S,4R,5R,6S)-4,5-diacetoxy-3-fluoro-6-(4-methyl-2-oxo-chromen-7-yl)oxy-tetrahydropyran-2-yl]methyl acetate (45)

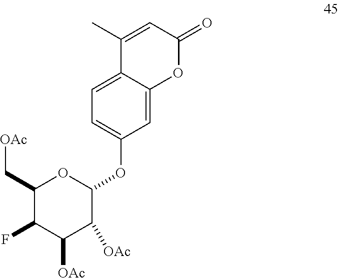 [SMILES: CC(=O)OC[C@H]1O[C@H](Oc2ccc3c(C)cc(=O)oc3c2)[C@H](OC(C)=O)[C@@H](OC(C)=O)[C@H]1F]

To [(2R,3S,4R,5R)-4,5-diacetoxy-6-bromo-3-fluoro-tetrahydropyran-2-yl]methyl acetate (379 mg, 1.02 mmol, 1.00 eq) (118) as a solution in dichloromethane was added tetrabutylammonium hydrogen sulfate (346 mg, 1.02 mmol, 1.00 eq), 4-methylumbelliferone (539 mg, 3.06 mmol, 3.00 eq) and sodium carbonate (1081 mg, 10.2 mmol, 10.0 eq). The resulting mixture was stirred vigorously overnight in the dark. TLC (50:50 ethyl acetate: cyclohexane) showed mostly unreacted bromide and excess 4-methylumbelliferone. LC/MS showed a trace of product (OA_UPLC1 _ A1266) Rt 1.54 min; m/z 467 [M+H]⁺. The aqueous layer was replaced with sodium carbonate (1081 mg, 10.2 mmol, 10.0 eq) and tetrabutylammonium bromide (329 mg, 1.02 mmol, 1.00 eq) and the resulting mixture stirred overnight. The dichloromethane layer was separated, washed with water, brine, dried (PTFE frit) and evaporated. The residue was dissolved in minimal dichloromethane and on standing a pale-yellow crystalline material precipitated (excess 4-methylumbelliferone). This was removed by filtration. The filtrate was concentrated and purified on silica using 5-65% ethyl acetate in cyclohexane as eluant. The product fractions were combined and evaporated to give a white solid (45) (300 mg, 63%). ¹H NMR (400 MHz, CDCl₃): d 7.52 (d, J=8.5 Hz, 1H), 6.97−6.93 (m, 2H), 6.20 (d, J=1.2 Hz, 1H), 5.58 (dd, J=8.0, 10.4 Hz, 1H), 5.13 (d, J=7.8 Hz, 1H), 5.12−5.02 (m, 1H), 4.94 (dd, J=2.4, 50.2 Hz, 1H), 4.42−4.28 (m, 2H), 4.02 (td, J=6.5, 25.9 Hz, 1H), 2.41 (d, J=1.2 Hz, 3H), 2.15 (s, 3H), 2.14 (s, 3H), 2.09 (s, 3H). LC/MS: Rt=4.20 min; m/z=467 [M+H]⁺.

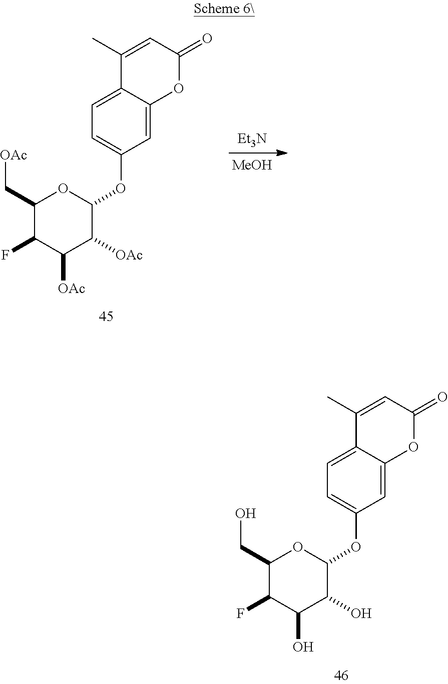 [SMILES: "Et3N" ; "MeOH" ; CC(=O)OC[C@H]1O[C@H](Oc2ccc3c(C)cc(=O)oc3c2)[C@H](OC(C)=O)[C@@H](OC(C)=O)[C@H]1F ; Cc1cc(=O)oc2cc(O[C@H]3O[C@H](CO)[C@H](F)[C@H](O)[C@H]3O)ccc12]

Scheme 6 illustrates the synthesis of compound 46.

### Example 10: 7-[(2S,3R,4R,5R,6R)-5-fluoro-3,4-dihydroxy-6-(hydroxymethyl)tetrahydropyran-2-yl]oxy-4-methyl-chromen-2-one (46)

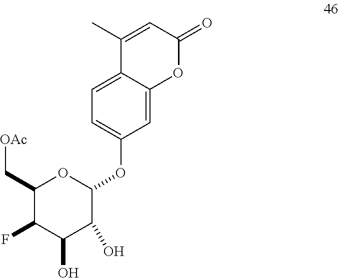 [SMILES: CC(=O)OC[C@H]1O[C@H](Oc2ccc3c(C)cc(=O)oc3c2)[C@H](O)[C@@H](O)[C@H]1F]

A mixture of [(2R,3S,4R,5R,6S)-4,5-diacetoxy-3-fluoro-6-(4-methyl-2-oxo-chromen-7-yl)oxy-tetrahydropyran-2-yl]methyl acetate (45) (120 mg, 0.257 mmol, 1.00 eq) and triethylamine (0.90 mL, 6.43 mmol, 25.0 eq) in methyl alcohol (8.00 mL) and water (1 mL) was stirred for 3 h. No visible dissolution occurred, and the mixture remained cloudy throughout. The white solid was isolated by filtration, washed with water, and air-dried followed by drying in vacuo overnight to give the product (46). ¹H NMR (400 MHz, DMSO) d 7.72 (d, J=8.7 Hz, 1H), 7.08−7.03 (m, 2H), 6.26 (d, J=1.2 Hz, 1H), 5.54 (d, J=4.9 Hz, 1H), 5.44 (d, J=5.5 Hz, 1H), 5.14 (d, J=7.3 Hz, 1H), 4.96 (t, J=5.6 Hz, 1H), 4.76−4.64 (m, 1H), 3.89 (ddd, J=6.8, 6.8, 28.7 Hz, 1H), 3.67−3.46 (m, 4H), 2.41 (d, J=1.1 Hz, 3H). LC/MS: Rt 2.35 min; m/z 340.9 [M+H]⁺.

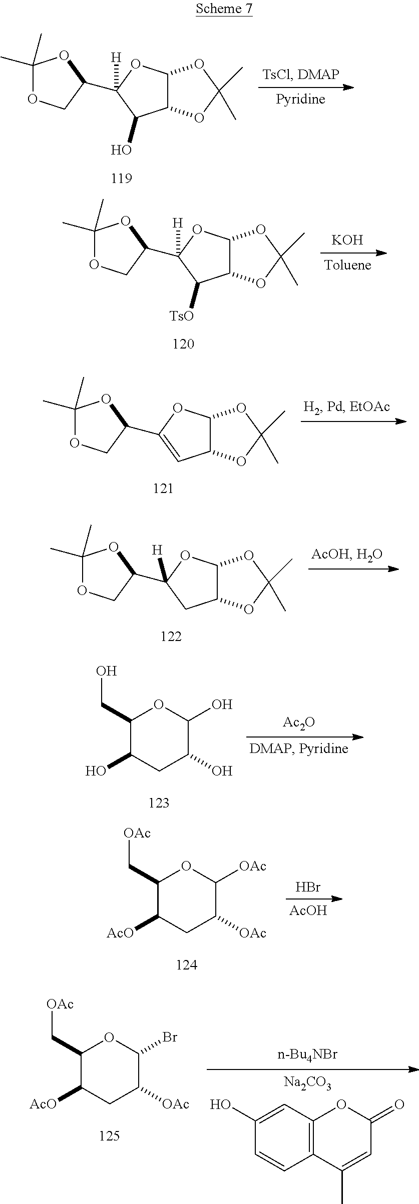

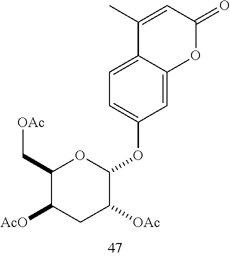 [SMILES: CC(=O)OC[C@H]1O[C@H](Oc2ccc3c(C)cc(=O)oc3c2)[C@H](OC(C)=O)C[C@H]1OC(C)=O]

Scheme 7 illustrates the synthesis of compound 47.

[(3aR,5R,6S,6aR)-5-[(4R)-2,2-dimethyl-1,3-dioxolan-4-yl]-2,2-dimethyl-3a,5,6,6a-tetrahydrofuro[2,3-d][1,3]dioxol-6-yl] 4-methylbenzenesulfonate (120)

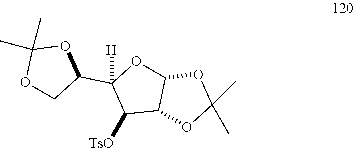

1,2:5,6-Di-O-isopropylidene-alpha-D-glucofuranose (119) (5000 mg, 19.2 mmol, 1.00 eq) was dissolved in pyridine (30 mL) and 4-(dimethylamino)pyridine (235 mg, 1.92 mmol, 0.1000 eq) was added followed by p-toluenesulfonyl chloride (7.32 g, 38.4 mmol, 2.00 eq). The pale-yellow reaction mixture was stirred at room temperature overnight. TLC analysis (EtOAc/cyclohexane 1:2) showed ˜1:1 starting material to product. Additional 4-(dimethylamino)pyridine (235 mg, 1.92 mmol, 0.1000 eq) was added and the mixture was stirred for 72 h and the solvent was removed in vacuo. The residue was dissolved in EtOAc and the solution washed with water (3× to remove residual pyridine), brine, dried and evaporated. The crude material was purified by silica chromatography (silica 120g, EtOAc in cyclohexane 0-20%) which removed unreacted TsCl and the product was eluted. Product fractions were combined and evaporated to give the title compound as a white crystalline substance (120) (7 g, 88%). ¹H NMR (400 MHz, CDCl₃): d 7.84 (d, J=8.3 Hz, 2H), 7.34 (d, J=8.1 Hz, 2H), 5.92 (d, J=3.7 Hz, 1H), 4.84 (d, J=3.8 Hz, 1H), 4.79 (d, J=2.4 Hz, 1H), 4.06 - 3.89 (m, 4H), 2.46 (s, 3H), 1.48 (s, 3H), 1.31 (s, 3H), 1.20 (s, 3H), 1.15 (s, 3H).
(3aR,6aR)-5-[(4R)-2,2-dimethyl-1,3-dioxolan-4-yl]-2,2-dimethyl-3a,6a-dihydrofuro[2,3-d][1,3]dioxole (121)

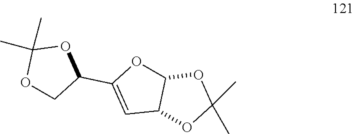

[(3aR,5R,6S,6aR)-5-[(4R)-2,2-dimethyl-1,3-dioxolan-4-yl]-2,2-dimethyl-3a,5,6,6a-tetrahydrofuro[2,3-d][1,3]dioxol-6-yl] 4-methylbenzenesulfonate (120) (7.00 g, 16.9 mmol, 1.00 eq) was dissolved in toluene (250 mL) and potassium hydroxide (2.94 g, 52.4 mmol, 3.10 eq) (crushed to a fine powder) was added. The reaction was heated under reflux for 5 h. TLC (25% EtOAc in cyclohexane) showed the product as the most non-polar spot with starting material just below and a more polar side product (unidentified). Heating was continued until all starting material was consumed. The reaction mixture was cooled to room temperature and water (250 mL) was added. The layers were separated, and the organic layer was washed with brine, dried (MgSO₄) and the solvent removed in vacuo to give a pale-yellow oil, which was purified by silica chromatography (80 g, eluting with EtOAc/cyclohexane 0-30%) to give the title compound (121) as a clear oil (2.9 g, 70%) which crystallized to give a white solid on standing. ¹H NMR (400MHz, CDCl₃): 6.08 (d, J=5.2 Hz, 1H), 5.32−5.29 (m, 1H), 5.25−5.24 (m, 1H), 4.60−4.57 (m, 1H), 4.15 (dd, J=6.8, 8.4 Hz, 1H), 3.97 (dd, J=5.7, 8.4 Hz, 1H), 1.47 (s, 6H), 1.45 (s, 3H), 1.39 (s, 3H).
(3aR,5R,6aR)-5-[(4R)-2,2-dimethyl-1,3-dioxolan-4-yl]-2,2-dimethyl-3a,5,6,6a-tetrahydrofuro[2,3-d][1,3]dioxole (122)

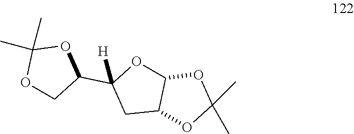

A solution of (3aR,6aR)-5-[(4R)-2,2-dimethyl-1,3-dioxolan-4-yl]-2,2-dimethyl-3a,6a-dihydrofuro [2,3-d][1,3]dioxole (121) (2.80 g, 11.6 mmol, 1.00 eq) in ethyl acetate (100 mL) was placed under argon. Palladium (10%, 1230 mg, 1.16 mmol, 0.100 eq) was moistened with ethyl acetate under CO₂ and added to the above solution. The mixture was evacuated and purged with argon (3×) and then stirred under a balloon of hydrogen overnight. TLC (25% EtOAc in cyclohexane) indicated all starting material had reacted and one major product (more polar than starting material) and a by-product were formed. The crude mixture was purged with argon and evacuated (3×) and the catalyst removed by filtration through Celite. The filtrate was concentrated, and the crude material was purified by column chromatography using 0-30% ethyl acetate in cyclohexane as eluant. Product fractions (located by Hanessian staining) were combined and evaporated to give the title compound (122) as a white crystalline solid (1.51 g, 53%). ¹H NMR (400 MHz, CDl₃): 5.80 (d, J=3.8 Hz, 1H), 4.75−4.71 (m, 1H), 4.47−4.41 (m, 1H), 4.14−4.08 (m, 1H), 4.05 (dd, J=6.6, 8.2 Hz, 1H), 3.61 (dd, J=6.9, 8.2 Hz, 1H), 2.21 (ddd, J=6.1, 8.3, 14.3 Hz, 1H), 1.82 (ddd, J=1.2, 3.9, 14.3 Hz, 1H), 1.57 (s, 3H), 1.45−1.44 (m, 3H), 1.37 (s, 3H), 1.33 (s, 3H).

(3R,5R,6R)-6-(hydroxymethyl)tetrahydropyran-2,3,5-triol (123)

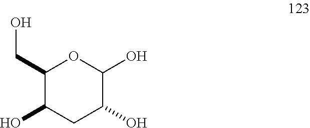 [SMILES: OC[C@H]1OC(O)[C@H](O)C[C@H]1O]

A mixture of acetic acid (20.195 mL) and water (20.20 mL) was added to (3aR,5R,6aR)-5-[(4R)-2,2-dimethyl-1,3-dioxolan-4-yl]-2,2-dimethyl-3a,5,6,6a-tetrahydrofuro[2,3-d][1,3]dioxole (122) (1.48 g, 6.06 mmol, 1.00 eq) and the resulting solution heated at 70° C. for 12 h. The solvent was evaporated, and the residue dried in vacuo to give 1.2 g of crude material as a viscous oil (123). ¹H NMR (400 MHz, CD₃OD, 263384) was consistent with a mixture of 3-4 isomeric products. No attempt to purify further was made due to the polarity of the compounds.

[(2R,3R,5R)-3,5,6-triacetoxytetrahydropyran-2-yl] methyl acetate (124)

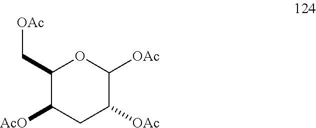 [SMILES: CC(=O)OC[C@H]1OC(OC(C)=O)[C@H](OC(C)=O)C[C@H]1OC(C)=O]

(3R,5R,6R)-6-(hydroxymethyl)tetrahydropyran-2,3,5-triol (123) (90% pure, 1.20 g, 6.58 mmol, 1.00 eq) was suspended in pyridine (21.93 mL) and acetic anhydride (3.7 mL, 39.5 mmol, 6.00 eq) and 4-(dimethylamino)pyridine (80 mg, 0.658 mmol, 0.100 eq) were added. Addition of DMAP led to a slight exotherm which persisted for ˜20 minutes. The pale-yellow solution was stirred for 1h. TLC (ethyl acetate-cyclohexane 1:1) showed one major product. The solution was concentrated in vacuo, the residue dissolved in ethyl acetate and washed with water (3×), dried (brine, sodium sulfate) and evaporated. The residue was purified on silica using 0-50% ethyl acetate in cyclohexane to give the product (0.62 g) as a clear oil. ¹H NMR was consistent with a mixture of 3-4 isomeric products. The oil was re-purified on silica (40 g, 15 μm ) using 0-50% TBDME in cyclohexane to give: (a) (124) (220 mg white solid, 10%): ¹H NMR (400 MHz, CDCl₃) consistent with desired product (axial acetate, pyran) d, 6.29 (d, J=3.1 Hz, 1H), 5.24−5.17 (m, 2H), 4.20−4.03 (m, 3H), 2.16 (s, 3H), 2.13 (s, 3H), 2.06 (s, 3H), 2.02 (s, 3H), 2.18−2.04 (m, 2H, partially hidden under acetates); and b) (124) (enriched sample was further purified by crystallization from ether) 200 mg 10% consistent with product (equatorial acetate, pyran) d , 5.71 (d, J=8.3 Hz, 1H), 5.14−5.02 (m, 2H), 4.21−4.00 (m, 3H), 2.45 (ddd, J=3.5, 5.1, 14.2 Hz, 1H), 2.13 (s, 6H), 2.06 (s, 3H), 2.05 (s, 3H), 1.80 (ddd, J=3.1, 11.3, 14.3 Hz, 1H).
[(2R,3R,5R)-3,5-diacetoxy-6-bromo-tetrahydropyran-2-yl]methyl acetate (125)

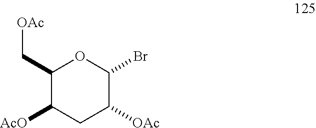 [SMILES: CC(=O)OC[C@H]1O[C@H](Br)[C@H](OC(C)=O)C[C@H]1OC(C)=O]

To a stirred solution of [(2R,3R,5R,6R)-3,5,6-triacetoxytetrahydropyran-2-yl]methyl acetate (124) (220 mg, 0.662 mmol, 1.00 eq) in dichloromethane (4.00 mL) at 0° C., was added 6 M Hydrogen bromide (2.6 mL, 15.5 mmol, 23.4 eq) as a 33 wt % solution in AcOH. The mixture was stirred at room temperature for 1 h and then neutralized at 0° C. with a saturated aqueous NaHCO3 solution (˜30 mL). The dichloromethane layer was separated and was not evaporated. TLC (50:50 ethyl acetate: hexanes) showed a less polar spot and all starting material had reacted. The crude bromide (125) was used as a solution in DCM for the next step without further purification.

### Example 11: [(2R,3R,5R,6S)-3,5-diacetoxy-6-(4-methyl-2-oxo-chromen-7-yl)oxy-tetrahydropyran-2-yl]methyl acetate (47)

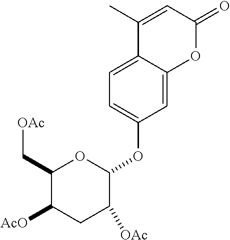 [SMILES: CC(=O)OC[C@H]1O[C@H](Oc2ccc3c(C)cc(=O)oc3c2)[C@H](OC(C)=O)C[C@H]1OC(C)=O]

To [(2R,3R,5R)-3,5-diacetoxy-6-bromo-tetrahydropyran-2-yl]methyl acetate (125) (220 mg, 0.623 mmol, 1.00 eq) crude as a solution in dichloromethane was added tetrabutylammonium bromide (201 mg, 0.623 mmol, 1.00 eq), 4-methylumbelliferone (329 mg, 1.87 mmol, 3.00 eq) and sodium carbonate (660 mg, 6.23 mmol, 10.0 eq). The resulting mixture was stirred vigorously overnight in the dark. TLC (50:50 ethyl acetate: cyclohexane) showed mostly product and excess 4-methylumbelliferone. LC/MS showed product (OA_UPLC1 _A1633) Rt 1.50 min; m/z 449.3 [M+H]⁺. The dichloromethane layer was separated, washed with water, brine, dried (PTFE frit) and evaporated. The residue was dissolved in minimal dichloromethane and on standing a pale-yellow crystalline material precipitated (excess 4-methylumbelliferone) which was removed by filtration. The filtrate was concentrated and purified on silica (12 μm) using 5-65% ethyl acetate in cyclohexane as eluant. The product fractions were combined and evaporated to give the product as a white solid, which was freeze-dried from acetonitrile water (1:5) as a suspension to give a white solid (230 mg, 82%). ¹H NMR (400 MHz, CDCl₃): d 7.52 (d, J=8.7 Hz, 1H), 7.01 (d, J=2.3 Hz, 1H), 6.96 (dd, J=2.5, 8.9 Hz, 1H), 6.19 (d, J=1.1 Hz, 1H), 5.29−5.14 (m, 3H), 4.21−4.09 (m, 3H), 2.51 (ddd, J=3.6, 5.1, 14.3 Hz, 1H), 2.42 (d, J=1.0 Hz, 3H), 2.15 (s, 3H), 2.10 (s, 3H), 2.09 (s, 3H), 1.92−1.83 (m, 1H). LC/MS Rt 1.49 min: m/z 449 [M+H ]⁺.

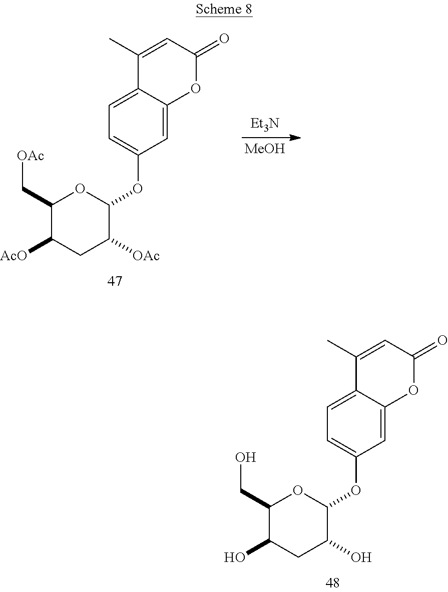 [SMILES: "Et3N" ; "MeOH" ; CC(=O)OC[C@H]1O[C@H](Oc2ccc3c(C)cc(=O)oc3c2)[C@H](OC(C)=O)C[C@H]1OC(C)=O ; Cc1cc(=O)oc2cc(O[C@H]3O[C@H](CO)[C@H](O)C[C@H]3O)ccc12]

Scheme 8 illustrates the synthesis of compound 48.

### Example 12: 7-[(2S,3R,5R,6R)-3,5-dihydroxy-6-(hydroxymethyl)tetrahydropyran-2-yl]oxy-4-methyl-chromen-2-one (48)

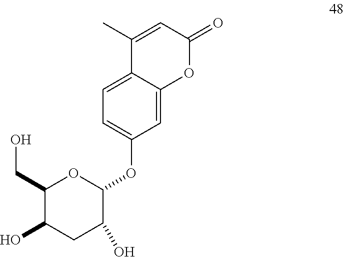 [SMILES: Cc1cc(=O)oc2cc(O[C@H]3O[C@H](CO)[C@H](O)C[C@H]3O)ccc12]

A mixture of [(2R,3R,5R,6S)-3,5-diacetoxy-6-(4-methyl-2-oxo-chromen-7-yl)oxy-tetrahydropyran-2-yl]methyl acetate (47) (120 mg, 0.268 mmol, 1.00 eq) and triethylamine (0.93 mL, 6.69 mmol, 25.0 eq) in methyl alcohol (8.00 mL) and water (1 mL) was stirred for 3 h. The mixture dissolved after a few minutes sonication and remained in solution throughout. The solution was stirred overnight. An additional aliquot of triethylamine was added (0.5 mL) and the solution stirred for 3 h. The solution was evaporated, and the residue stirred in water for 1 h. The white solid was isolated by filtration and the solid dried in vacuo at 40° C. to provide (48). ¹H NMR (400 MHz, MeOD-d₄): d 7.70 (d, J=8.8 Hz, 1H), 7.14−7.08 (m, 2H), 6.19 (d, J=1.0 Hz, 1H), 5.00 (d, J=7.6 Hz, 1H), 4.07−3.97 (m, 2H), 3.80−3.72 (m, 3H), 2.45 (d, J=0.9 Hz, 3H), 2.26 (ddd, J=3.1, 5.2, 13.6 Hz, 1H), 1.77 (ddd, J=2.7, 11.6, 13.9 Hz, 1H) was consistent with product. LC/MS: Rt=2.20 min; m/z=323 [M+H]⁺, 345 [M+Na]⁺.

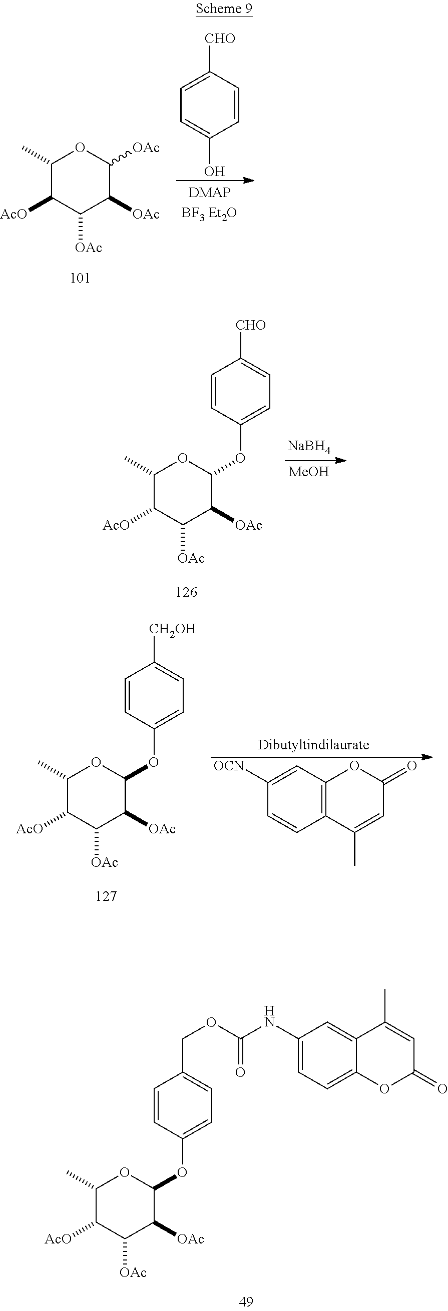

Scheme 9 illustrates the synthesis of compound 49.

[(2S,3R,4R,5S,6S)-4,5-diacetoxy-6-(4-formylphenoxy)-2-methyl-tetrahydropyran-3-yl]acetate (126)

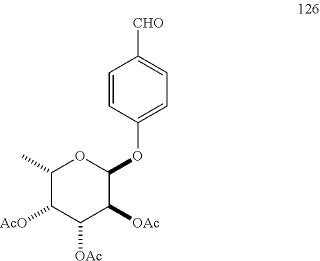 [SMILES: CC(=O)O[C@H]1[C@H](OC(C)=O)[C@H](Oc2ccc(C=O)cc2)O[C@@H](C)[C@H]1OC(C)=O]

A mixture of 1,2,3,4-tetra-o-acetyl-alpha-1-fucopyranose (3.00 g, 9.03 mmol, 1.00 eq) (101) and 4-hydroxybenzaldehyde (2.20 g, 18.1 mmol, 2.00 eq) was suspended in 1,2-dichloroethane (40 ml) under argon and 4-(dimethylamino)pyridine (4.41 g, 36.1 mmol, 4.00 eq) was added and the mixture stirred for 15 minutes to ensure dissolution. The solution was cooled in ice-water under argon. Boron trifluoride diethyl etherate (14 mL, 0.112 mol, 12.4 eq) was added dropwise, giving a pale brown solution. The resulting solution was heated at 63° C. (external) for 3 h. TLC (20% ethyl acetate in toluene) showed product, acetate SM, (trans product) and excess DMAP.BF₃ adduct. The brown solution was cooled and neutralized by adding slowly to saturated aqueous NaHCO₃ until effervescence ceased. The product was extracted with dichloromethane. The dichloromethane extracts were washed with IN NaOH to remove unreacted phenol, brine, dried (PTFE) and concentrated. The crude product was purified by chromatography on silica (40 g, 50 tm), eluting with 0-20% ethyl acetate in toluene to elute first the desired product (126) (1.30 g, 2.64 mmol, 29%) as a yellow oil which semi-crystallized on standing. ¹H NMR (400 MHz, CDCl₃) d 9.93 (s, 1H), 7.86 (d, J=9.0 Hz, 2H), 7.21−7.17 (m, 2H), 5.86 (d, J=3.7 Hz, 1H), 5.58 (dd, J=3.3, 11.0 Hz, 1H), 5.37 (dd, J=0.8, 3.3 Hz, 1H), 5.31 (dd, J=3.5, 11.0 Hz, 1H), 4.22 (q, J=6.5 Hz, 1H), 2.20 (s, 3H), 2.06 (s, 3H), 2.04 (s, 3H), 1.13 (d, J=6.5 Hz, 3H).

[(2S,3R,4R,5S,6S)-4,5-diacetoxy-6-14-(hydroxymethyl)phenoxyl-2-methyl-tetrahydropyran-3-yl] acetate (127)

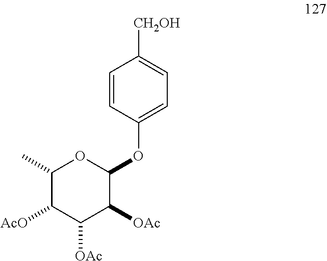 [SMILES: CC(=O)O[C@H]1[C@H](OC(C)=O)[C@H](Oc2ccc(CO)cc2)O[C@@H](C)[C@H]1OC(C)=O]

A solution of [(2S,3R,4R,5S,6S)-4,5-diacetoxy-6-(4-formylphenoxy)-2-methyl-tetrahydropyran-3-yl] acetate (126) (80%, 1.30 g, 2.64 mmol, 1.00 eq) in dichloromethane (2.00 mL) and methyl alcohol (18.00 mL) was cooled in ice-water. Sodium borohydride (100 mg, 2.64 mmol, 1.00 eq) was added and the solution stirred for 30 minutes until the yellow coloration disappeared. TLC (ethyl acetate: cyclohexane 1:1) showed the disappearance of starting material and formation of a more polar substance. The mixture was quenched by the addition of 1 M hydrogen chloride (2.6 mL, 2.64 mmol, 1.00 eq). The solvent was evaporated, and the crude material was dispersed between water and dichloromethane. The organic extract was washed with brine, dried (PTFE) and evaporated to give the product as a white foam dried in vacuo. The crude material was purified on silica using 0-50% ethyl acetate in cyclohexane to give the product (127) (880 mg, 2.22 mmol, 84%) as a white foam. ¹H NMR (400 MHz, CDCl₃): d 7.32 (d, J=8.4 Hz, 2H), 7.05 (d, J=8.7 Hz, 2H), 5.74 (d, J=3.7 Hz, 1H), 5.58 (dd, J=3.4, 10.9 Hz, 1H), 5.36 (d, J=3.1 Hz, 1H), 5.28 (dd, J=3.6, 10.9 Hz, 1H), 4.64 (d, J=5.8 Hz, 2H), 4.27 (q, J=6.6 Hz, 1H), 2.20 (s, 3H), 2.06 (s, 3H), 2.03 (s, 3H), 1.60 (t, J=5.9 Hz, 1H), 1.12 (d, J=6.5 Hz, 3H).

### Example 13: [(2S,3R,4R,5S,6S)-4,5-diacetoxy-2-methyl-6-[4-[(4-methyl-2-oxo-chromen-7-yl)carbamoyloxymethyl]phenoxy]tetrahydropyran-3-yl]acetate (49)

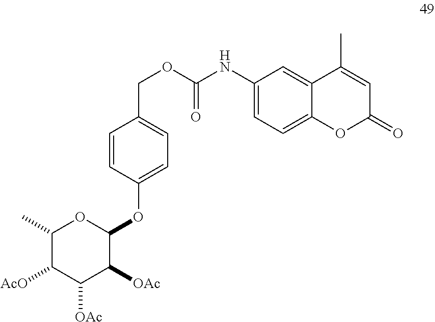 [SMILES: CC(=O)O[C@H]1[C@H](OC(C)=O)[C@H](Oc2ccc(COC(=O)Nc3ccc4oc(=O)cc(C)c4c3)cc2)O[C@@H](C)[C@H]1OC(C)=O]

To a solution of [(2S,3R,4R,5S,6S)-4,5-diacetoxy-6-[4-(hydroxymethyl)phenoxy]-2-methyl-tetrahydropyran-3-yl] acetate (126) (100 mg, 0.251 mmol, 1.00 eq) in dry tetrahydrofuran (3.00 mL) was added 7-isocyanato-4-methyl-chromen-2-one (60 mg, 0.300 mmol, 1.00 eq) and the resulting suspension was stirred under argon for 5 minutes. Dibutyltin dilaurate (0.0089 mL, 0.0150 mmol, 0.0500 eq) was added and the suspension slowly dissolved. The reaction mixture was stirred under argon for -2 hours. The solution was concentrated and purified by chromatography on silica (12 g) using 0-50% ethyl acetate in cyclohexane. This gave the product (49) containing ˜15% by-product amine. The compound was dissolved in dichloromethane and purified on silica (15 μm, 12 g) using 0-3% MeOH in dichloromethane. The eluate was concentrated and recrystallized from ethyl acetate/cyclohexane to give the pure product. The crystals were re-dissolved in acetonitrile (0.5 mL), the solution diluted with water (2.0 mL) and the white suspension was freeze-dried overnight to give the product (49) (25 mg, 0.0418 mmol, 17%) as a white solid. ¹H NMR (400 MHz, CDCl₃): d 7.52 (d, J=8.7 Hz, 1H), 7.43 (d, J=2.7 Hz, 1H), 7.39−7.33 (m, 3H), 7.10−7.05 (m, 2H), 6.88 (s, 1H), 6.19 (d, J=1.2 Hz, 1H), 5.75 (d, J=3.7 Hz, 1H), 5.58 (dd, J=3.4, 10.9 Hz, 1H), 5.36 (dd, J=1.1, 3.3 Hz, 1H), 5.27 (dd, J=3.6, 10.9 Hz, 1H), 5.18 (s, 2H), 4.26 (q, J=6.6 Hz, 1H), 2.40 (d, J=1.2 Hz, 3H), 2.19 (s, 3H), 2.05 (s, 3H), 2.03 (s, 3H), 1.12 (d, J=6.5 Hz, 3H). LC/MS Rt 4.74 min; m/z 596.1.

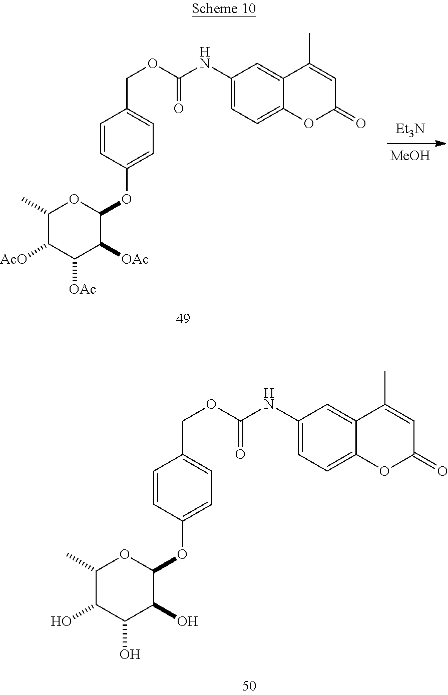 [SMILES: "Et3N" ; "MeOH" ; CC(=O)O[C@H]1[C@H](OC(C)=O)[C@H](Oc2ccc(COC(=O)Nc3ccc4oc(=O)cc(C)c4c3)cc2)O[C@@H](C)[C@H]1OC(C)=O ; Cc1cc(=O)oc2ccc(NC(=O)OCc3ccc(O[C@@H]4O[C@@H](C)[C@@H](O)[C@@H](O)[C@@H]4O)cc3)cc12]

Scheme 10 illustrates the synthesis of compound 50.

### Example 14: [4-[(2S,3S,4R,5S,6S)-3,4,5-trihydroxy-6-methyl-tetrahydropyran-2-yl]oxyphenyl]methyl N-(4-methyl-2-oxo-chromen-7-yl)carbamate (50)

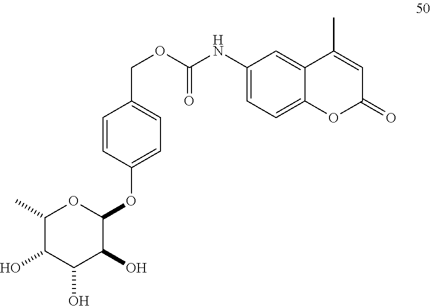 [SMILES: Cc1cc(=O)oc2ccc(NC(=O)OCc3ccc(O[C@@H]4O[C@@H](C)[C@@H](O)[C@@H](O)[C@@H]4O)cc3)cc12]

[(2S,3R,4R,5S,6S)-4,5-diacetoxy-2-methyl-6-[4-[(4-methyl-2-oxo-chromen-7-yl)carbamoyloxymethyl]phenoxy]tetrahydropyran-3-yl] acetate (49) (70 mg, 0.117 mmol, 1.00 eq) was suspended in methyl alcohol (5.00 mL) and water (0.50 mL) and the mixture sonicated until all SM dissolved. Triethylamine (0.50 mL, 3.59 mmol, 30.6 eq) was added and the solution stirred overnight at room temperature. A white precipitate formed and was removed by filtration and air-dried to give the product [4-[(2S,3S,4R,5S,6S)-3,4,5-trihydroxy-6-methyl-tetrahydropyran-2-yl]oxyphenyl]methyl N-(4-methyl-2-oxo-chromen-7-yl)carbamate (50) (25 mg, 0.0524 mmol, 45%). ¹HNMIR (400 MHz, DMSO-d₆): d 10.24 (s, 1H), 7.69 (d, J=8.7 Hz, 1H), 7.55 (d, J=2.0 Hz, 1H), 7.43−7.36 (m, 3H), 7.09−7.04 (m, 2H), 6.24 (d, J=1.1 Hz, 1H), 5.41 (d, J=3.1 Hz, 1H), 5.13−5.11 (m, 2H), 4.84 (d, J=5.7 Hz, 1H), 4.68 (d, J=5.2 Hz, 1H), 4.57 (d, J=4.5 Hz, 1H), 3.87 (q, J=6.6 Hz, 1H), 3.78−3.71 (m, 2H), 3.58−3.54 (m, 1H), 2.39 (s, 3H), 1.04 (d, J=6.5 Hz, 3H). Rt 3.36 min; m/z 494.0 (M+Na)⁺.

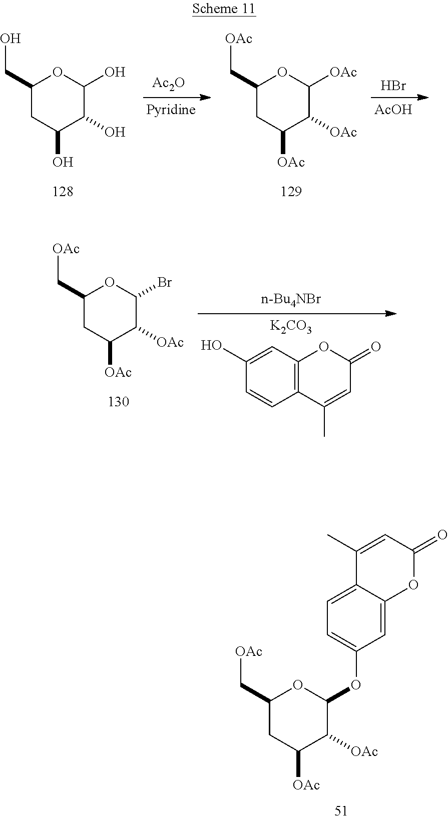

Scheme 11 illustrates the synthesis of compound 51.

(2R,3R,4S,6S)-6-(acetoxymethyl)tetrahydro-2H-pyran-2,3,4-triyl triacetate (129)

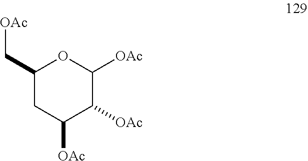 [SMILES: CC(=O)OC[C@@H]1C[C@H](OC(C)=O)[C@@H](OC(C)=O)C(OC(C)=O)O1]

To a solution of (3R,4S,6S)-6-(hydroxymethyl)tetrahydropyran-2,3,4-triol (500 mg, 3.05 mmol, 1.00 eq) (128) and 4-(dimethylamino)pyridine (37 mg, 0.305 mmol, 0.100 eq) in pyridine (10 mL) at 0° C. was added acetic anhydride (4.3 mL, 45.7 mmol, 15.0 eq) over a period of ten minutes and the reaction mixture was stirred at 0° C. for 2.5 h. The reaction mixture was concentrated to a minimum volume and the remaining pyridine co-evaporated from toluene. The oily residue was re-dissolved in toluene and washed with 1 M HCl, water and brine. The organic phase was dried (Na₂SO₄), filtered and concentrated to give the title compound (129) (993 mg, 98%). ¹H NMR (300 MHz, CDCl₃) d, 5.69−5.65 (m, 1H), 5.09−4.99 (m, 2H), 4.19−4.15 (m, 2H), 3.96−3.87 (m, 1H), 2.23−2.16 (m, 1H), 2.12 (s, 3H), 2.10 (s, 3H), 2.06 (s, 6H), 1.73−1.59 (m, 1H).

(2R,3R,4S,6S)-6-(acetoxymethyl)-2-bromotetrahydro-2H-pyran-3,4-diyl diacetate (130)

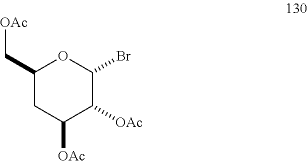 [SMILES: CC(=O)OC[C@@H]1C[C@H](OC(C)=O)[C@@H](OC(C)=O)[C@@H](Br)O1]

To a reaction vessel protected from light, were added [(2S,4S,5R)-4,5,6-triacetoxytetrahydropyran-2-yl]methyl acetate (200 mg, 0.602 mmol, 1.00 eq) (129) and dichloromethane (5 mL). The flask was maintained at 0° C. and hydrogen bromide in acetic acid (33%) (0.6 mL) was added slowly under a nitrogen atmosphere. The reaction mixture was allowed to stir at room temperature and monitored by TLC. After 3 h TLC (1:1 cyclohexane: ethyl acetate) showed the expected product at R_{f} 0.60. The crude reaction mixture was added portion-wise into a beaker containing a mixture of sodium bicarbonate (1.1 g) and ice-water (8 mL) and mixed vigorously (evolving gas) for 5 minutes. The organic phase was separated, and the aqueous phase was further extracted with dichloromethane (30 mL). The combined organic phases were dried (Na₂SO₄), filtered and the volatiles evaporated to give the title compound (130) (200 mg, 94%) as a colorless oil. ¹H NMR (300 MHz, CDCl₃) d, 6.66 (d, J=3.9 Hz, 1H), 4.79 (dd, J=3.9, 9.9 Hz, 1H), 4.43−4.34 (m, 1H), 4.18 (d, J=4.6 Hz, 2H), 2.34−2.25 (m, 1H), 2.13 (s, 3H), 2.12 (s, 3H), 2.07 (s, 3H), 1.71 (ddd, J=12.0, 12.0, 12.0 Hz, 1H).

### Example 15: [(2S,4S,5R,6S)-4,5-diacetoxy-6-(4-methyl-2-oxo-chromen-7-yl)oxy-tetrahydropyran-2-yl]methyl acetate (51)

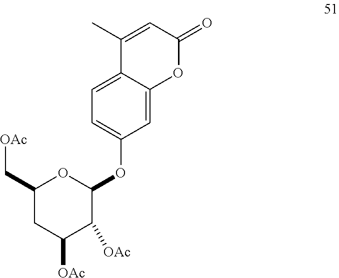 [SMILES: CC(=O)OC[C@@H]1C[C@H](OC(C)=O)[C@@H](OC(C)=O)[C@H](Oc2ccc3c(C)cc(=O)oc3c2)O1]

To a solution of 4-methylumbelliferone (100 mg, 0.566 mmol, 1.00 eq) in dichloromethane (0.6 mL) was added a solution of potassium carbonate (94 mg, 0.680 mmol, 1.20 eq) in water (1.2 mL) and tetrabutylammonium bromide (91 mg, 0.283 mmol, 0.500 eq). The mixture was stirred at room temperature for 10 minutes at and a solution of [(2S,4S,5R,6R)-4,5-diacetoxy-6-bromo-tetrahydropyran-2-yl]methyl acetate (130) (200 mg, 0.566 mmol, 1.00 eq) in dichloromethane (0.6 mL) was added. The reaction mixture was vigorously stirred at room temperature and monitored by TLC (1:1 ethyl acetate: hexane). After 3.5 h the reaction mixture was diluted with dichloromethane and the aqueous layer separated. The organic layer was washed with water, dried (Na₂SO₄), filtered and the volatiles evaporated to give a crude product as a white foam (285 mg) which was purified by flash chromatography using a 24 g, 15-micron silica cartridge (eluted with cyclohexane: ethyl acetate (2-60%)) to give after evaporation the product (51) as a white foam (98 mg). Recrystallization from cyclohexane/ethyl acetate gave a crystalline title product (51) (71 mg). ¹H NMR (300 MHz, CDCl₃): d 7.53 (d, J=8.8 Hz, 1H), 6.99−6.92 (m, 2H), 6.20 (d, J=1.2 Hz, 1H), 5.26−5.07 (m, 3H), 4.27−4.16 (m, 2H), 4.03−3.94 (m, 1H), 2.42 (d, J=1.2 Hz, 3H), 2.25 (ddd, J=2.0, 5.2, 12.8 Hz, 1H), 2.14 (s, 3H), 2.10 (s, 3H), 2.09 (s, 3H), 1.73 (dd, J=11.6, 24.1 Hz, 1H). LC/MS: Rt=4.12 min; m/z=449 [M+H]⁺.

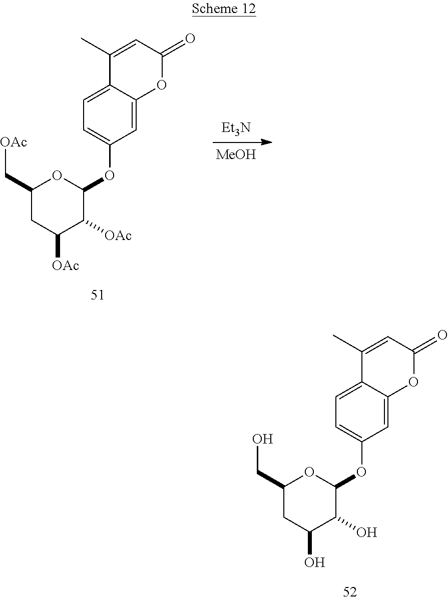 [SMILES: "Et3N" ; "MeOH" ; CC(=O)OC[C@@H]1C[C@H](OC(C)=O)[C@@H](OC(C)=O)[C@H](Oc2ccc3c(C)cc(=O)oc3c2)O1 ; Cc1cc(=O)oc2cc(O[C@@H]3O[C@H](CO)C[C@H](O)[C@H]3O)ccc12]

Scheme 12 illustrates the synthesis of compound 52.

### Example 16: 7[(2S,3R,4S,6S)-3,4-dihydroxy-6-(hydroxymethyl)tetrahydropyran-2-yl]oxy-4-methyl-chromen-2-one (52)

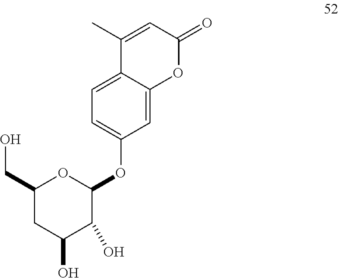 [SMILES: Cc1cc(=O)oc2cc(O[C@@H]3O[C@H](CO)C[C@H](O)[C@H]3O)ccc12]

A mixture of [(2S,4S,5R,65)-4,5-diacetoxy-6-(4-methyl-2-oxo-chromen-7-yl)oxy-tetrahydropyran-2-yl]methyl acetate (51) (52 mg, 0.116 mmol, 1.00 eq) in methyl alcohol (1.2 mL), triethylamine (0.40 mL, 2.90 mmol, 25.0 eq) and water (0.15 mL) was stirred at room temperature for 21 h. The reaction mixture was concentrated to a minimum volume and then diluted with water. The solid was collected by filtration, washed with water, and dried in vacuo at 35 ° C. for 24 h to give the title compound (52) (28.1 mg, 75%) as a white solid. ¹H NMR (400 MHz, Me0D): d 7.71 (d, J=8.8 Hz, 1H), 7.13−7.06 (m, 2H), 6.20 (d, J=1.1 Hz, 1H), 4.98 (d, J=7.7 Hz, 1H), 3.82−3.70 (m, 2H), 3.63−3.59 (m, 2H), 3.39 (dd, J=7.8, 9.0 Hz, 1H), 2.45 (d, J=1.2 Hz, 3H), 2.03−1.97 (m, 1H), 1.49 (dd, J=11.7, 24.4 Hz, 1H). Rt=2.25 min; m/z=323 [M+H]⁺.

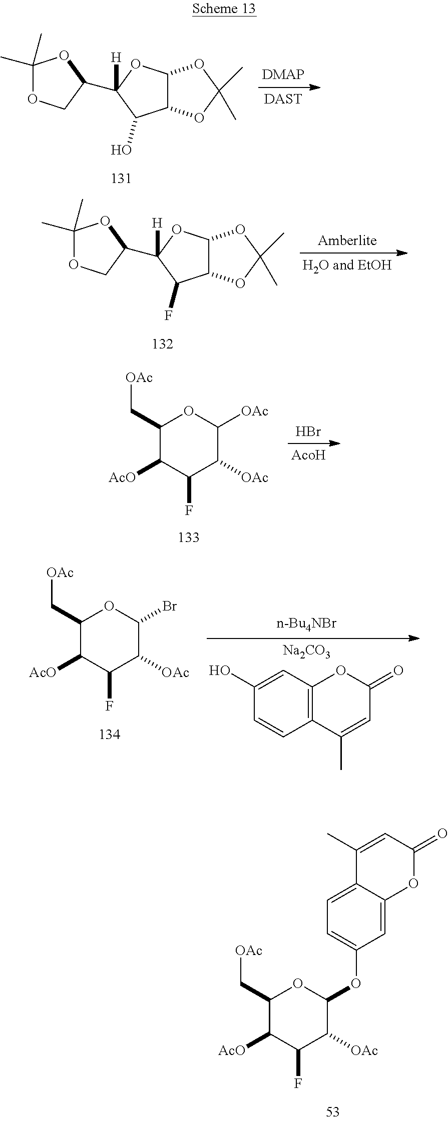

Scheme 13 illustrates the synthesis of compound 53.

(3aR,5S,6S,6aS)-5-[(4R)-2,2-dimethyl-1,3-dioxolan-4-yl]-6-fluoro-2,2-dimethyl-3a,5,6,6a-tetrahydrofuro[2,3-d][1,3]dioxole (132)

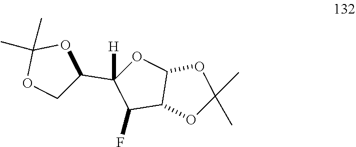

1,2:5,6-Di-O-isopropylidene-α-D-gulofuranose (131) (3.00 g, 11.5 mmol, 1 eq.) was dissolved in dichloromethane (20 mL) and the solution cooled to −10° C. 4-(dimethylamino)pyridine (2.82 g, 23.1 mmol, 2.00 eq) was added to the reaction mixture followed by slow addition of (diethylamino)sulfur trifluoride (3.0 mL, 23.1 mmol, 2.00 eq). The reaction mixture was allowed to warm to room temperature and monitored by TLC (7:3 cyclohexane: ethyl acetate). After 24 h, a new spot at Rf 0.7 was observed and the reaction mixture was cooled to −20° C., MeOH was slowly added while the temperature was maintained between −20° C. and −10° C. The mixture was allowed to warm to room temperature and then was partitioned between aqueous saturated NaHCO₃ and dichloromethane. The organic layer was passed through a phase separator cartridge and the solvent evaporated to give a crude product (4.1 g) purified by flash chromatography using cyclohexane: ethyl acetate (1 to 35%) to give the title compound (132) (3.0 g, 99%) as a colorless oil which later crystallized. ¹H NMR (300 MHz, CDCl₃): d, 5.94 (d, J=3.8 Hz, 1H), 4.85 (dd, J=3.6, 40.8 Hz, 1H), 4.74 (dd, J=3.9, 3.9 Hz, 1H), 4.39−4.32 (m, 1H), 4.20−4.06 (m, 2H), 3.84 (dd, J=6.6, 8.3 Hz, 1H), 1.56 (s, 3H), 1.47 (s, 3H), 1.40 (s, 3H), 1.37 (s, 3H).
[(2R,3S,4S,5S)-3,5,6-triacetoxy-4-fluoro-tetrahydropyran-2-yl]methyl acetate (133)

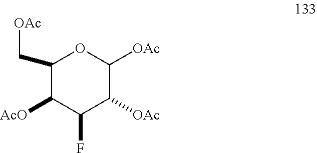 [SMILES: CC(=O)OC[C@H]1OC(OC(C)=O)[C@H](OC(C)=O)[C@@H](F)[C@H]1OC(C)=O]

3-Deoxy-3- fluoro-1,2:5,6-di-O-isopropylidene-a-D-galactofuranose (3aR,5S,6S,6aS)-5-[(4R)-2,2-dimethyl-1,3-dioxolan-4-yl]-6-fluoro-2,2-dimethyl-3a,5,6,6a-tetrahydrofuro[2,3-d][1,3]dioxole (132) (380 mg, 1.45 mmol, 1.00 eq) was dissolved in ethanol (16 mL) and water (33.00 mL). Amberlite IR-120 (H+) (ca. 3 mL) was added, and the reaction mixture was stirred at 60-65° C. and monitored by TLC (1:1 ethyl acetate: cyclohexane). After 4 h, TLC indicated the disappearance of starting material. The reaction mixture was filtered and concentrated in vacuo to give a crude product (216 mg) as the expected pyranose which was confirmed by ¹H NMR. The crude material was dissolved in dry pyridine (3.00 mL), cooled to 0° C. under a nitrogen atmosphere and treated with acetic anhydride (0.68 mL, 7.24 mmol, 5.00 eq). The reaction mixture was allowed to warm to room temperature and monitored b TLC (3:7 ethyl acetate: cyclohexane). After 24 h, TLC showed a major sport at Rf 0.5 for the expected product. The reaction mixture was concentrated, and the residue evaporated from toluene (5 mL). The crude oil was partitioned between ethyl acetate and aqueous saturated NaHCO₃. The organic layer was separated, dried (Na₂SO₄), filtered and the volatiles evaporated to give an oil (385 mg) which was purified by flash chromatography (12 g silica cartridge eluted with cyclohexane: ethyl acetate (2 to 50%)) gave the title compound (133) as an anomeric mixture (258 mg) of a colorless dense oil. ¹H NMIR (400 MHz, CDC13): d 6.42 (t, J=4.2, 1H), 5.72−5.68 (m, 1H), 5.67 (d, J=8.3 Hz, 1H), 5.62 (m, 1H), 5.48−5.39 (m, 2H), 4.97 (ddd, J=3.8, 10.2, 48.4 Hz, 1H), 4.71 (ddd, J=3.8, 9.7, 47.3 Hz, 1H), 4.33−4.29 (m, 1H), 4.25−4.07 (m, 4H), 4.00 (tt, J=1.5, 6.5 Hz, 1H), 2.21 (s, 3H), 2.20 (s, 3H), 2.18 (s, 3H), 2.16 (s, 3H), 2.13 (s, 3H), 2.10 (s, 3H), 2.09 (s, 3H), 2.08 (s, 3H).
[(2R,3S,4S,5R)-3,5-diacetoxy-6-bromo-4-fluoro-tetrahydropyran-2-yl]methyl acetate (134)

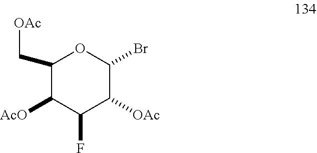 [SMILES: CC(=O)OC[C@H]1O[C@H](Br)[C@H](OC(C)=O)[C@@H](F)[C@H]1OC(C)=O]

To a solution of [(2R,3S,4S,5S)-3,5,6-triacetoxy-4-fluoro-tetrahydropyran-2-yl]methyl acetate (133) (258 mg, 0.737 mmol, 1.00 eq) in dry dichloromethane (7 mL) at 0° C. was slowly added hydrogen bromide in acetic acid (33%) (0.7 mL) and the reaction mixture was stirred at room temperature and monitored by TLC (1:1 ethyl acetate: cyclohexane). After 3 h, TLC showed the product at Rf 0.8. The reaction mixture was diluted with dichloromethane and quenched by addition into an ice-water/NaHCO₃ solution (9 mL/1.3 g). The organic phase was passed through a phase separator cartridge and the volatiles evaporated to give the title compound (134) that was taken directly into the next synthetic step.

### Example 17: [(2R,3S,4S,5S,6S)-3,5-diacetoxy-4-fluoro-6-(4-methyl-2-oxo-chromen-7-yl)oxy-tetrahydropyran-2-yl]methyl acetate (53)

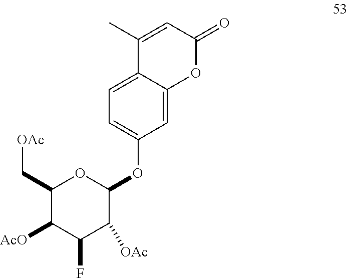 [SMILES: CC(=O)OC[C@H]1O[C@@H](Oc2ccc3c(C)cc(=O)oc3c2)[C@H](OC(C)=O)[C@@H](F)[C@H]1OC(C)=O]

To a stirred suspension of 4-methylumbelliferone (130 mg, 0.736 mmol, 1.00 eq), tetrabutylammonium bromide (119 mg, 0.368 mmol, 0.500 eq) and a solution of potassium carbonate (112 mg, 0.810 mmol, 1.10 eq) in water (1.6 mL) and dichloromethane (0.8 mL) was added a solution of crude [(2R,3S,4S,5R)-3,5-diacetoxy-6-bromo-4-fluoro-tetrahydropyran-2-yl]methyl acetate (134) (273 mg, 0.736 mmol, 1.00 eq) in dichloromethane (0.8 mL). The reaction mixture was intensively stirred at room temperature overnight and then partitioned between water and dichloromethane. The organic layer was passed through a phase separator cartridge and the volatiles evaporated to give a crude product (320 mg). Purification by flash chromatography, (12 g, 15-micron silica cartridge eluted with dichloromethane: ethyl acetate (0-20%)) gave an impure product. A second purification by flash chromatography,(12 g, 15-micron, silica cartridge eluted dichloromethane: ethyl acetate from 5-20%) gave the title compound (53) (68 mg) as a white solid. ¹H NMR (400 MHz, CDCl₃) d 7.56−7.53 (m, 1H), 6.99−6.95 (m, 2H), 6.22 (d, J=1.2 Hz, 1H), 5.68−5.59 (m, 2H), 5.09 (d, J=7.9 Hz, 1H), 4.77 (ddd, J=3.8, 9.8, 47.2 Hz, 1H), 4.29−4.19 (m, 2H), 4.10−4.04 (m, 1H), 2.44 (d, J=1.2 Hz, 3H), 2.23 (s, 3H), 2.17 (s, 3H), 2.15 (s, 3H). LC/MS: Rt=4.17 min; m/z=467.1 [M+H]⁺.

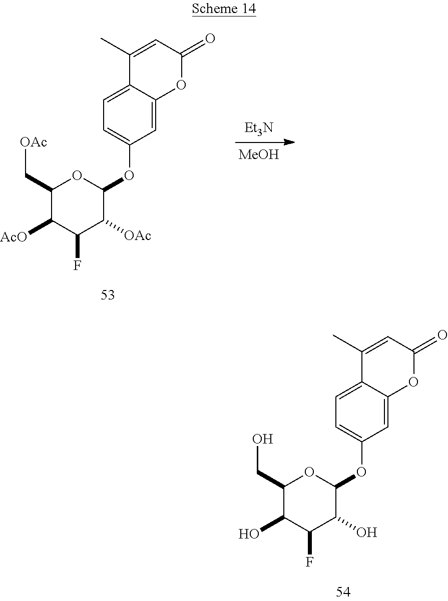 [SMILES: "Et3N" ; "MeOH" ; CC(=O)OC[C@H]1O[C@@H](Oc2ccc3c(C)cc(=O)oc3c2)[C@H](OC(C)=O)[C@@H](F)[C@H]1OC(C)=O ; Cc1cc(=O)oc2cc(O[C@@H]3O[C@H](CO)[C@H](O)[C@H](F)[C@H]3O)ccc12]

Scheme 14 illustrates the synthesis of compound 54.

### Example 18: 7-[(2S,3S,4S,5S,6R)-4-fluoro-3,5-dihydroxy-6-(hydroxymethyl)tetrahydropyran-2-yl]oxy-4-methyl-chromen-2-one (54)

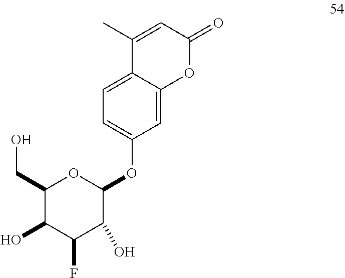 [SMILES: Cc1cc(=O)oc2cc(O[C@@H]3O[C@H](CO)[C@H](O)[C@H](F)[C@H]3O)ccc12]

[(2R,3S,4S,5S,6S)-3,5-diacetoxy-4-fluoro-6-(4-methyl-2-oxo-chromen-7-yl)oxy-tetrahydropyran-2-yl]methyl acetate (53) (43 mg, 0.0922 mmol, 1.00 eq) was suspended in methyl alcohol (2.2 mL) and treated with water (0.3 mL) and triethylamine (0.32 mL, 2.30 mmol, 25.0 eq). The suspension was stirred at room temperature and monitored by LCMS. After 24 h, more triethylamine (0.32 mL, 2.30 mmol, 25.0 eq) and water (0.3 mL) were added, and the mixture stirred at room temperature for 24 h. The solid was collected by filtration and dried at 40° C. to give a solid (3 mg) which was combined with mother liquors and concentrated. The residue was triturated with MeOH, the solid was collected by filtration and dried at 40° C. to give (21 mg, 66%) as a white solid (54). ¹H NMR (400 MHz, MeOD): d 7.74 (d, J=8.8 Hz, 1H), 7.16−7.11 (m, 2H), 6.23 (d, J=1.2 Hz, 1H), 5.06 (d, J=7.7 Hz, 1H), 4.53 (ddd, J=3.4, 9.5, 48.7 Hz, 1H), 4.21−4.08 (m, 2H), 3.81−3.77 (m, 3H), 2.48 (d, J=1.2 Hz, 3H). LC/MS: Rt=2.25 min; m/z=341.0 [M+H]⁺.

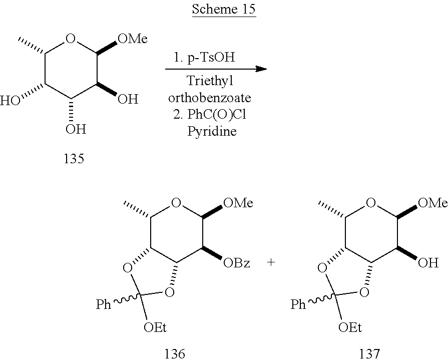

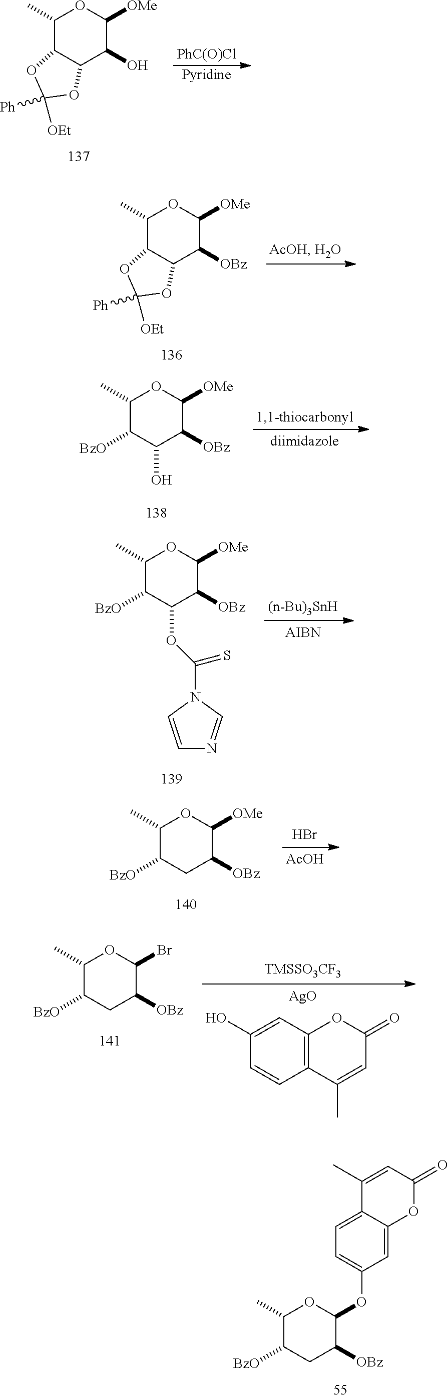

Scheme 15 illustrates the synthesis of compound 55.

[(3aR,4S,7S,7aR)-2-ethoxy-6-methoxy-4-methyl-2-phenyl-4,6,7,7a-tetrahydro-3aH-[1,3]dioxolo[4,5-c]pyran-7-yl]benzoate (136) and (3aR,4S,7S,7aS)-2-ethoxy-6-methoxy-4-methyl-2-phenyl-4,6,7,7a-tetrahydro-3aH-[1,3]dioxolo[4,5-c]pyran-7-ol and (137)

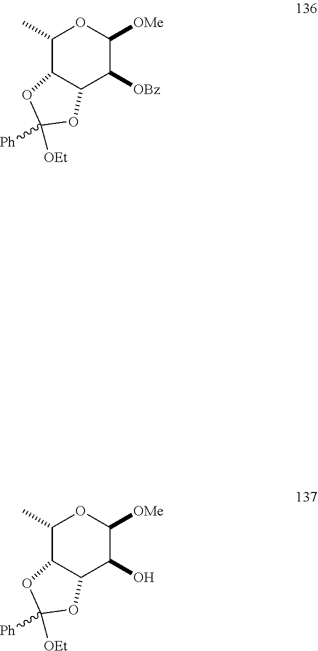

To a solution of (3S,4R,5S,6S)-2-methoxy-6-methyl-tetrahydropyran-3,4,5-triol (5.00 g, 28.1 mmol, 1.00 eq.) (135) in anhydrous dichloromethane (100 mL) was added a catalytic amount of p-toluenesulfonic acid monohydrate (534 mg, 2.81 mmol, 0.1 eq) and triethyl orthobenzoate (7.9 mL, 35.1 mmol, 1.25 eq.). The reaction mixture was stirred at room temperature for 30 minutes and then transferred slowly via canula to a solution of benzoyl chloride (4.1 mL, 35.1 mmol, 1.25 eq.) in pyridine (50 mL). After 24 h, the reaction mixture was diluted with dichloromethane and then poured slowly over an ice-cooled aqueous solution of saturated NaHCO₃. The organic layer was separated, dried (Na₂SO₄), filtered and volatiles evaporated to give a crude product as a colorless oil (14.3 g). Most of the crude was taken into the next step to further convert the unreacted (3aR,4S,7S,7aS)-2-ethoxy-6-methoxy-4-methyl-2-phenyl-4,6,7,7a-tetrahydro-3aH-[1,3]dioxolo[4,5-c]pyran-7-ol (137) into the desired [(3aR,4S,7S,7aR)-2-ethoxy-6-methoxy-4-methyl-2-phenyl-4,6,7,7a-tetrahydro-3aH-[1,3]dioxolo[4,5-c]pyran-7-yl] benzoate (136). For characterization purposes, a small amount of the crude product (290 mg) was taken for purification by flash chromatography, 4 g silica cartridge eluted with cyclohexane: ethyl acetate from 1-100%) to give two separated compounds: epimeric mixture of [(3aR,4S,7S,7aR)-2-ethoxy-6-methoxy-4-methyl-2-phenyl-4,6,7,7a-tetrahydro-3aH-[1,3]dioxolo[4,5-c]pyran-7-yl]benzoate (56 mg) (136) as a colorless oil, and epimeric mixture of (3aR,4S,7S,7aS)-2-ethoxy-6-methoxy-4-rnethyl-2-phenyl-4,6,7,7a-tetrahydro-3aH-[1,3]dioxolo[4,5-c]pyran-7-ol (109 mg) (137) as a colorless oil.

[3aR,4S,7S,7aR)-2-ethoxy-6-methoxy-4-methyl-2-phenyl-4,6,7,7a-tetrahydro-3aH-[1,3]dioxolo [4,5-c]pyran-7-yl ]benzoate (136)

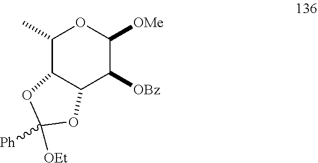

To a solution of crude (3aR,4S,7S,7aS)-2-ethoxy-6-methoxy-4-methyl-2-phenyl-4,6,7,7a-tetrahydro-3aH-[1,3]dioxolo[4,5-c]pyran-7-ol (12.56 g, 40.5 mmol, 1.00 eq.) (137) in pyridine (36 mL) at 0° C. was added benzoyl chloride (5.2 mL, 44.5 mmol, 1.10 eq.). The reaction mixture was allowed to stir overnight at room temperature, concentrated and the residue partitioned between ethyl acetate and aqueous saturated NaHCO₃. The organic layer was separated and washed with water, dried (Na₂SO₄), filtered and the volatiles evaporated to give a crude product that was purified by flash chromatography, (110 g silica cartridge eluted with cyclohexane: ethyl acetate 0-30%) to give the title compound (136) (11.70 g, 70%) as a colorless oil.

(2S,3S,4R,5S) -5-benzoyloxy-4-hydroxy-6-methoxy-2-methyl-tetrahydropyran3-yl]benzoate (138)

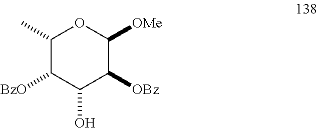

[(3aR,4S,7S,7aR)-2-ethoxy-6-methoxy-4-methyl-2-phenyl-4,6,7,7a-tetrahydro-3aH-[1,3]dioxolo[4,5-c]pyran-7-yl] benzoate (136) (11.77 g, 28.4 mmol, 1.00 eq.) was treated with acetic acid (56 mL) and water (14.00 mL) and the reaction mixture was stirred overnight at room temperature. The reaction mixture was concentrated, and the residue dissolved in ethyl acetate and poured into aqueous saturated NaHCO₃. The organic phase was separated and dried (Na₂SO₄), filtered and the volatiles evaporated to give a crude product (12.5 g). Recrystallization from ethyl acetate/dichloromethane, gave the title compound (138) (3.3 g, 30%) as a white solid. The mother liquors were further recrystallized from ethyl acetate: dichloromethane to give a second crop of the desired product (138) (2.46 g, 22%) as a white solid. The second mother liquors were concentrated to give an oily product (4.6 g) purified by flash chromatography, (80 silica cartridge eluted with cyclohexane: ethyl acetate (2-35%)) to give after evaporation more of the title compound (138) (1 g, 9%) as a white solid. ¹H NMR (300 MHz, CDCl₃): d 8.19−8.08 (m, 4H), 7.63−7.44 (m, 6H), 5.58 (dd, J=1.2, 3.6 Hz, 1H), 5.37 (dd, J=3.6, 10.4 Hz, 1H), 5.13 (d, J=3.7 Hz, 1H), 4.54−4.46 (m, 1H), 4.30−4.22 (m, 1H), 3.46 (s, 3H), 2.22 (d, J=6.1 Hz, 1H), 1.27 (d, J=6.6 Hz, 3H).
[(2S,3R,4R,5S)-5-benzoyloxy-4-(imidazole-1-carbothioyloxy)-6-methoxy-2-methyl-tetrahydropyran-3-yl]benzoate (139)

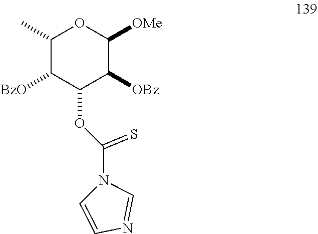

[(2S,3S,4R,5S)-5-benzoyloxy-4-hydroxy-6-methoxy-2-methyl-tetrahydropyran-3-yl] benzoate (138) (3.30 g, 8.54 mmol, 1.00 eq.) was dissolved in toluene (30 mL) and treated with 1,1 [symbol] thiocarbonyldiimidazole (1826 mg, 10.2 mmol, 1.20 eq). The reaction was heated at 70° C. and monitored by LCMS. After 4 h, the reaction mixture was concentrated to give a crude product purified by flash chromatography, (40 g silica cartridge eluted with cyclohexane: ethyl acetate (1-35%)) to give the title compound (139) (4.08 g, 95%) as white solid. ¹H NMR (300 MHz, CDCl₃): d 8.13−8.09 (m, 3H), 8.02−7.98 (m, 2H), 7.69−7.62 (m, 1H), 7.60−7.49 (m, 3H), 7.46−7.39 (m, 2H), 7.36−7.34 (m, 1H), 6.86−6.84 (m, 1H), 6.41 (dd, J=3.3, 10.7 Hz, 1H), 5.90−5.88 (m, 1H), 5.79 (dd, J=3.8, 10.5 Hz, 1H), 5.23 (d, J=3.6 Hz, 1H), 4.45−4.38 (m, 1H), 3.52 (s, 3H), 1.32 (d, J=6.5 Hz, 3H).
(2S,3S,5S,6R)-5-benzoyloxy-6-methoxy-2-methyl-tetrahydropyran-3-yl] benzoate (140)

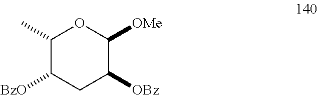

To a solution of [(2S,3R,4R,5S,6R)-5-benzoyloxy-4-(imidazole-1-carbothioyloxy)-6-methoxy-2-methyl-tetrahydropyran-3-yl] benzoate (139) (1.00 g, 2.01 mmol, 1.00 eq) in dry toluene (40 mL) at 55° C. was added 2,2 [symbol] atzobis(2-methylpropionitrile) (0.083 g, 0.503 mmol, 0.25 eq) and tributyltin hydride (1.1 mL, 4.03 mmol, 2.00 eq). The reaction mixture was heated at 90° C. for 24 h, concentrated and the residue purified by flash chromatography (24 g silica cartridge eluted with cyclohexane: ethyl acetate from 0 to 35%) to give the title compound (140) (567 mg,72%) as a dense oil. ¹H NMR (300 MHz, CDCl₃): d 8.17−8.03 (m, 4H), 7.62−7.42 (m, 6H), 5.45−5.34 (m, 2H), 5.08 (d, J=3.4 Hz, 1H), 4.21 (dq, J=1.3, 6.6 Hz, 1H), 3.49 (s, 3H), 2.45 (ddd, J=12.9, 12.9, 3.0 Hz, 1H), 2.32−2.25 (m, 1H), 1.25 (d, J=6.6 Hz, 3H).

[(2S,3S,5S,6S)-5-benzoyloxy-6-bromo-2-methyl-tetrahydropyran-3-yl]benzoate (141)

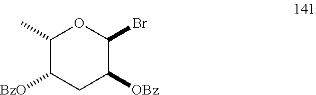

To a reaction vessel protected from light were added [(2S,3S,5S,6R)-5-benzoyloxy-6-methoxy-2-methyl-tetrahydropyran-3-yl] benzoate (140) (261 mg, 0.705 mmol, 1.00 eq) in dry dichloromethane (6 mL). The solution was cooled to 0° C. and hydrogen bromide in acetic acid (33%) (0.7 mL) was slowly added under a nitrogen atmosphere. The reaction mixture was allowed to stir at 0° C. for 1. H, then at room temperature and was monitored by TLC (ethyl acetate: cyclohexane 3:7). After 3 h, the crude reaction mixture was added portion-wise into a beaker containing a mixture of sodium bicarbonate (1.3 g) and ice-water (9 mL) and mixed vigorously (evolving gas) for 5 minutes. The organic phase was separated, and the aqueous phase was further extracted with dichloromethane. The combined organic extracts were dried (Na₂SO₄), filtered and the volatiles were removed under reduced pressure to give the title compound (295 mg) as a colorless oil which was taken directly into the next step.

### Example 19: [(2S,3S,5S,6S)-5-benzoyloxy-2-methyl-6-(4-methyl-2-oxo-chromen-7-yl)oxy-tetrahydropyran-3-yl]benzoate (55)

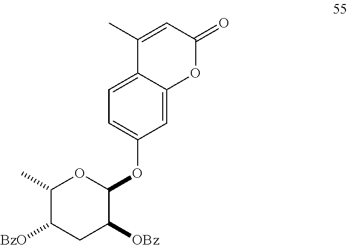

A mixture of [(2S,3S,5S)-5-benzoyloxy-6-bromo-2-methyl-tetrahydropyran-3-yl] benzoate (141) (170 mg, 0.405 mmol, 1.00 eq), 4-methylumbelliferone (71 mg, 0.405 mmol, 1.00 eq) and freshly activated MS 4A (1 g) in dry dichloromethane (5 mL) was stirred under argon at room temperature for 1h. The mixture was cooled to 0° C. and silver oxide (282 mg, 1.22 mmol, 3.00 eq) and trimethylsilyl trifluoromethanesulfonate (0.018 mL, 0.101 mmol, 0.250 eq) was added, and the resulting mixture was stirred for 24 h at room temperature and monitored by LCMS. The reaction mixture was passed through Celite and the filtrate was partitioned between water and dichloromethane. The organic phase was separated through a phase separation cartridge to give after evaporation of a crude product (180 mg). Purification by flash chromatography, ((12 g, 15-micron cartridge) eluted with dichloromethane: ethyl acetate (0-7%)) gave a solid (51 mg) which was further purified by recrystallization from cyclohexane and ethyl acetate to give the title compound (55) (30 mg, 14%) as a white solid. ¹H NMR (400 MHz, CDC₃): d 8.18−8.14 (m, 2H), 8.03−8.00 (m, 2H), 7.64−7.40 (m, 7H), 7.14 (d, J=2.6 Hz, 1H), 7.09 (dd, J=2.5, 8.6 Hz, 1H), 6.17 (d, J=1.2 Hz, 1H), 5.92 (d, J=3.2 Hz, 1H), 5.59−5.52 (m, 1H), 5.41−5.40 (m, 1H), 4.30−4.26 (m, 1H), 2.65 (ddd, J=13.1, 13.1, 2.9 Hz, 1H), 2.49−2.42 (m, 1H), 2.40 (d, J=1.1 Hz, 3H), 1.24−1.21 (m, 3H). LC/MS: Rt=5.8 min; m/z=515.1 [M+H]⁺.

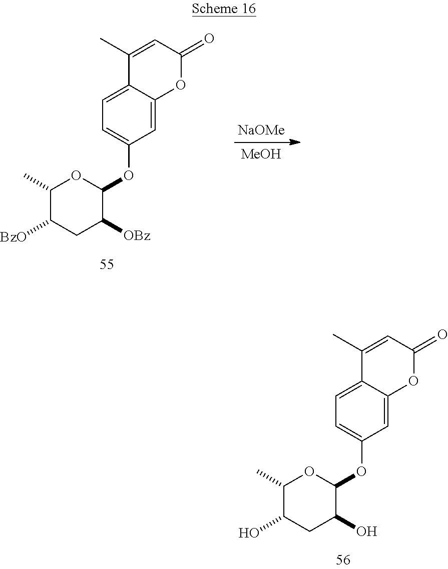

Scheme 16 illustrates the synthesis of compound 56.

### Example 20: 7-[(2S,3S,5S,6S)-3,5-dihydroxy-6-methyl-tetrahydropyran-2-yl]oxy-4-methyl-chromen-2-one (56)

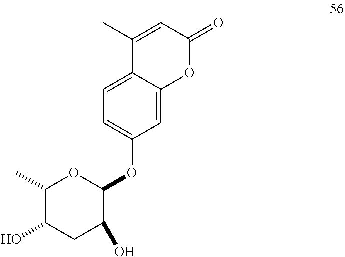 [SMILES: Cc1cc(=O)oc2cc(O[C@@H]3O[C@@H](C)[C@@H](O)C[C@@H]3O)ccc12]

To a solution of [(2S,3S,5S,6S)-5-benzoyloxy-2-methyl-6-(4-methyl-2-oxo-chromen-7-yl)oxy-tetrahydropyran-3-yl] benzoate (55) (70 mg, 0.136 mmol, 1.00 eq) in dry methyl alcohol (2 mL) was added sodium methoxide in methanol 30% (0.016 mL, 0.272 mmol, 2.00 eq). The reaction mixture was allowed to stir at room temperature for 24 h and monitored by LCMS. The reaction mixture was quenched by slow addition of a CO₂ pellet and concentrated to give a crude product purified by flash chromatography, eluted with cyclohexane: ethyl acetate (20-100%) to give the title compound (56) (20 mg, 30%) as a white solid. ¹H NMR (400 MHz, MeOD): d 7.73 (d, J=8.8 Hz, 1H), 7.20−7.13 (m, 2H), 6.21 (d, J=1.2 Hz, 1H), 5.56 (d, J=3.4 Hz, 1H), 4.22−4.16 (m, 1H), 3.95−3.89 (m, 1H), 3.80 (s, 1H), 2.48 (d, J=1.2 Hz, 3H), 2.20 (ddd, J=12.7, 12.7, 2.9 Hz, 1H), 2.12−2.04 (m, 1H), 1.15 (d, J=6.6 Hz, 3H). LC/MS: Rt=2.80 min; m/z=307 [M+H]⁺.

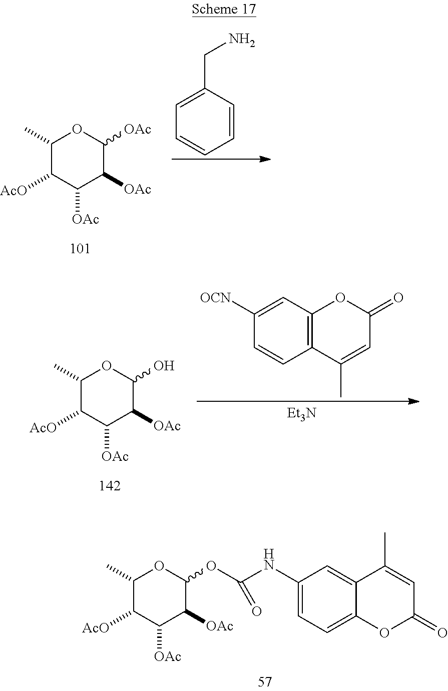

Scheme 17 illustrates the synthesis of compound 57.

[(2S,3R,4R,5S)-4,5-diacetoxy-6-hydroxy-2-methyl-tetrahydropyran-3-yl]acetate (142)

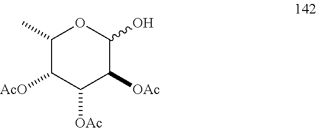 [SMILES: CC(=O)O[C@@H]1[C@H](OC(C)=O)[C@H](C)OC(O)[C@H]1OC(C)=O]

To a solution of 1,2,3,4-tetra-O-acetyl-alpha-L-fucopyranose (101) (1.00 g, 3.01 mmol, 1.00 eq) in tetrahydrofuran (10 mL) was added benzylamine (0.49 mL, 4.51 mmol, 1.50 eq) and the mixture stirred at room temperature and monitored by TLC (ethyl acetate: cyclohexane 1:1). After 12 h, the mixture was diluted with ethyl acetate and water. The organic layer was washed with 1M HCl, brine, dried (Na₂SO₄) and concentrated to give a crude product which was purified by flash chromatography on 24 g silica cartridge (eluting with cyclohexane: ethyl acetate (0-50%)) to provide the title compound (142) (anomeric mixture 4:1) (412 mg, 47%) as a brown solid.

### Example 21: [(2S,3R,4R, 5S,6S)-4,5-diacetoxy-2-methyl-6-[(4-methyl-2-oxo-chromen-7-yl)carbamoyloxy]tetrahydropyran-3-yl]acetate (57)

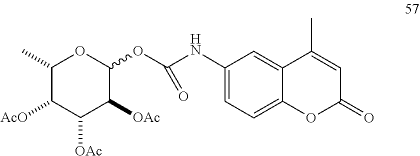 [SMILES: CC(=O)O[C@@H]1[C@H](OC(C)=O)[C@H](C)OC(OC(=O)Nc2ccc3oc(=O)cc(C)c3c2)[C@H]1OC(C)=O]

A solution of [(2S,3R,4R,5S)-4,5-diacetoxy-6-hydroxy-2-methyl-tetrahydropyran-3-yl]acetate (142) (70 mg, 0.241 mmol, 1.00 eq) was dissolved in dry dichloromethane (3 mL) and then cooled to 0° C. Triethylamine (0.034 mL, 0.241 mmol, 1.00 eq) was added followed by slow addition of 7-isocyanato-4-methyl-chromen-2-one (49 mg, 0.241 mmol, 1.00 eq). The reaction mixture was allowed to stir at 0° C. for 10 minutes, then warmed up to room temperature and monitored by TLC (1:1 cyclohexane: ethyl acetate). After 18 h, the reaction mixture was partitioned between 1M NaOH and dichloromethane. The organic layer was separated and washed with water, then passed through a phase separation cartridge and the volatiles evaporated. The crude residue was purified by flash chromatography (12 g silica cartridge, 15 micron, eluted with (cyclohexane: ethyl acetate 0-50%)) to give a mixture of anomers (70 mg) with no separation. Purification by reverse phase chromatography, 80 g C18 column 15-micron, eluted with water/acetonitrile (+0.1% HCO₂H) 50-70% gave a first eluting peak (12 mg) (the β-anomer) and the second eluting peak (29 mg) the a-anomer, the title compound (57). Data for the title compound: ¹H NMR (400 MHz, CDCl₃): d 7.47 (d, J=8.6 Hz, 1H), 7.41−7.35 (m, 2H), 7.05 (s, 1H), 6.27 (d, J=3.3 Hz, 1H), 6.14 (d, J=1.2 Hz, 1H), 5.36−5.26 (m, 3H), 4.22 (q, J=6.5 Hz, 1H), 2.33 (d, J=1.2 Hz, 3H), 2.11 (s, 3H), 1.95 (s, 3H), 1.93 (s, 3H), 1.10 (d, J=6.5 Hz, 3H) and LC/MS: Rt=4.24 min; m/z=492 [M+H]⁺.

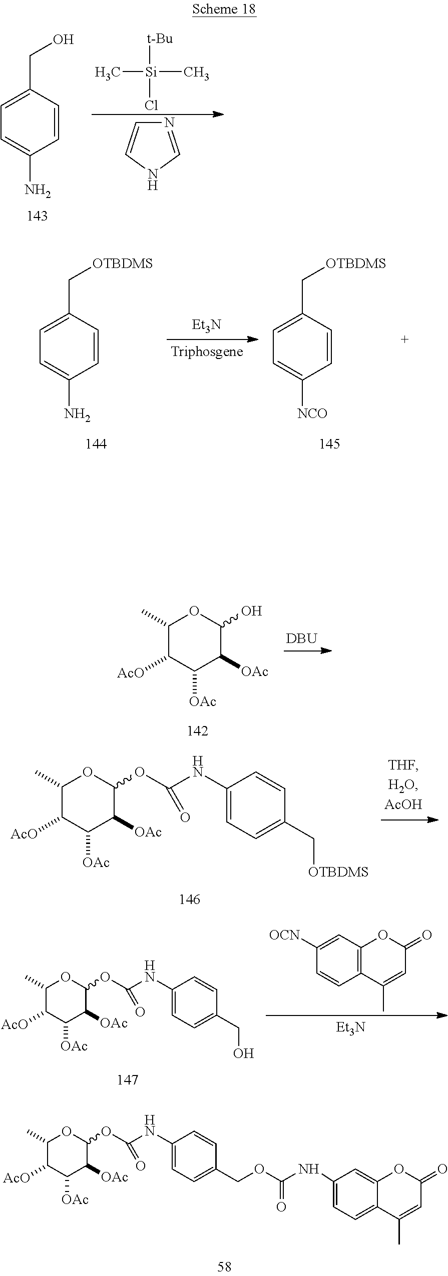

Scheme 18 illustrates the synthesis of compound 58.

4-[[tert-butyl(dimethyl)silyl]oxymethyl]aniline (144)

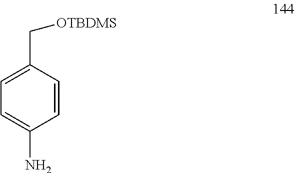 [SMILES: CC(C)(C)[Si](C)(C)OCc1ccc(N)cc1]

To a solution of 4-aminobenzyl alcohol (143) (1.00 g, 8.12 mmol, 1.00 eq) in dichloromethane (15 mL) were added tert-butyldimethylsilyl chloride (1224 mg, 8.12 mmol, 1.00 eq) and imidazole (564 mg, 8.28 mmol, 1.02 eq). The reaction mixture was stirred at room temperature and monitored by LCMS. After 18 h, the reaction mixture was partitioned between brine and dichloromethane. The organic phase was passed through a phase separation cartridge and the solvent was evaporated to give the title compound (144) (1.9 g, 99%) as an oil, ¹H NMR (300 MHz, CDCl₃) d 7.03 (d, J=7.8 Hz, 2H), 6.58 (d, J=7.5 Hz, 2H), 4.54 (s, 2H), 3.58−3.46 (m, 2H), 0.84 (s, 9H), -0.00 (s, 6H).

tert-butyl-[(4-isocyanatophenyl)methoxy]dimethylsilane (145)

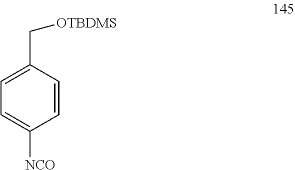

4-[[tert-butyl(dimethyl)silyl]oxymethyl]aniline (100%, 200 mg, 0.842 mmol, 1.00 eq) was dissolved in dry toluene (6 mL) and then treated with triethylamine (0.12 mL, 0.885 mmol, 1.05 eq). The solution was heated to 70° C., treated with triphosgene (100 mg, 0.337 mmol, 0.400 eq) and heated continued at 70° C. for 3h. The reaction mixture was cooled to room temperature, the solid precipitate was removed by filtration and the filtrate concentrated to give the title compound (145) (155 mg, 70%) as a beige oil used directly into the next synthetic step. ¹H NMR (300 MHz, CDCl₃) d, 7.18 (d, J=5.6 Hz, 2H), 6.95 (d, J=8.1 Hz, 2H), 4.60 (s, 2H), 0.84 (s, 9H), −0.01 (s, 6H).
[(2S,3R,4R,5S,6S)-4,5-diacetoxy-6-[[4-[[tert-butyl(dimethyl)silyl]oxymethyl]phenyl]carbamoyloxy]-2-methyl-tetrahydropyran-3-yl]acetate (146)

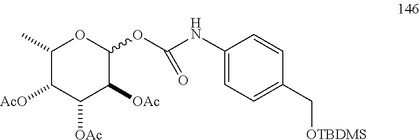 [SMILES: CC(=O)O[C@@H]1[C@H](OC(C)=O)[C@H](C)OC(OC(=O)Nc2ccc(CO[Si](C)(C)C(C)(C)C)cc2)[C@H]1OC(C)=O]

To a solution of [(2S,3R,4R,5S)-4,5-diacetoxy-6-hydroxy-2-methyl-tetrahydropyran-3-yl] acetate (142) (171 mg, 0.588 mmol, 1.00 eq) in dichloromethane (5 mL) was added a solution of tert-butyl-[(4-isocyanatophenyl)methoxy]-dimethyl-silane (145) (155 mg, 0.588 mmol, 1.00 eq) in dry dichloromethane (5 mL) followed by 1,8-diazabicyclo[5.4.0]undec-7-ene (0.026 mL, 0.177 mmol, 0.300 eq). The reaction mixture was stirred at room temperature and monitored by TLC. After 18 h the solution was partitioned between dichloromethane and water. The organic layer was passed through a phase separation cartridge, and the volatiles evaporated to give a crude product which was by flash chromatography, (12g silica cartridge eluted with cyclohexane: ethyl acetate (0-50%)) gave mainly the title compound (146) (150 mg, 46%) as a yellow solid. ¹H NMR (300 MHz, CDCl₃) d 7.38 (d, J=8.5 Hz, 2H), 7.29 (d, J=8.7 Hz, 2H), 6.71−6.71 (m, 1H), 6.34 (s, 1H), 5.36 (d, J=11.7 Hz, 3H), 4.69 (s, 2H), 4.28 (d, J=5.8 Hz, 1H), 2.19 (s, 3H), 2.03 (s, 3H), 2.01 (s, 3H), 1.17 (d, J=6.3 Hz, 3H), 0.93 (s, 9H), 0.08 (s, 6H).

[(2S,3R,4R,5S,6S)-4,5-diacetoxy-6-[[4-(hydroxymethyl)phenyl]carbamoyloxyl]-2-methyl-tetrahydropyran-3-yl] acetate (147)

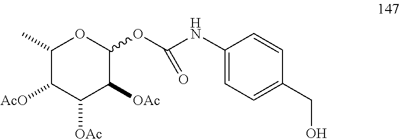 [SMILES: CC(=O)O[C@@H]1[C@H](OC(C)=O)[C@H](C)OC(OC(=O)Nc2ccc(CO)cc2)[C@H]1OC(C)=O]

[(2S,3R,4R,5S,6S)-4,5-diacetoxy-6-[[4-[[tert-butyl(dimethyl)silyl]oxymethyl]phenyl]carbamoyloxy]-2-methyl-tetrahydropyran-3-yl] acetate (146) (140 mg, 0.253 mmol, 1.00 eq) was stirred in a mixture of THF:H₂O:AcOH (1:1:1) (15 mL) and the reaction was followed by TLC. After 3 h, the reaction mixture was diluted with water and concentrated to a minimum volume. The aqueous residue was diluted with dichloromethane, the organic extracts were separated and washed with saturated aqueous NaHCO₃ until gas evolution ceased. The organic layer was dried (Na₂SO₄), filtered and the volatiles evaporated to give the title compound (147) (110 mg, 99%) as a pale-yellow solid. ¹H NMR (300 MHz, CDCl₃) d 7.42 (d, J=8.4 Hz, 2H), 7.34 (d, J=8.0 Hz, 2H), 6.77 (s, 1H), 6.35 (d, J=2.7 Hz, 1H), 5.41−5.32 (m, 3H), 4.66 (d, J=4.5 Hz, 2H), 4.32−4.25 (m, 1H), 2.20 (s, 3H), 2.03 (s, 3H), 2.02 (s, 3H), 1.66−1.60 (m, 1H), 1.18 (d, J=6.7 Hz, 3H).

### Example 22: [(2S,3R,4R,5S,6S)-4,5-diacetoxy-2-methyl-6-[[4-[(4-methyl-2-oxo-chromen-7- yl)carbamoyloxymethyl]phenyl]carbamoyloxy]tetrahydropyran-3-yl]acetate (58)

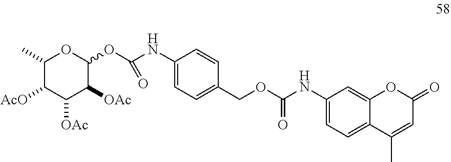 [SMILES: CC(=O)O[C@@H]1[C@H](OC(C)=O)[C@H](C)OC(OC(=O)Nc2ccc(COC(=O)Nc3ccc4c(C)cc(=O)oc4c3)cc2)[C@H]1OC(C)=O]

To a solution of [(2S,3R,4R,5S,6S)-4,5-diacetoxy-6-[[4-(hydroxymethyl)phenyl]carbamoyloxy]-2-methyl-tetrahydropyran-3-yl] acetate (147) (58 mg, 0.132 mmol, 1.00 eq) and triethylamine (0.018 mL, 0.132 mmol, 1.00 eq) in dry dichloromethane (3.00 mL) at 0° C., was added 7-isocyanato-4-methyl-chromen-2-one (27 mg, 0.132 mmol, 1.00 eq). The reaction mixture allowed to stir at 0° C. for 10 minutes, then at room temperature and monitored by LCMS. After 3 h, the reaction mixture was diluted with dichloromethane and washed with 1M NaOH. The organic layer was separated and washed with water, dried (Na₂SO₄) and volatiles evaporated to give a crude product (68 mg). Purification by reverse phase chromatography, 80 g C18 column 15-micron eluted with water/acetonitrile (+0.1% HCO₂H) (40-70%) provided the title compound (58) (29 mg, 85%) as a white solid. ¹H NMR (400 MHz, CDCl₃) d 7.52 (d, J=8.7 Hz, 1H), 7.49−7.33 (m, 6H), 6.91 (s, 1H), 6.82 (s, 1H), 6.35 (d, J=3.0 Hz, 1H), 6.19 (d, J=1.1 Hz, 1H), 5.43−5.32 (m, 3H), 5.19 (s, 2H), 4.32−4.24 (m, 1H), 2.40 (d, J=1.3 Hz, 3H), 2.19 (s, 3H), 2.03 (s, 3H), 2.02 (s, 3H), 1.18 (d, J=6.5 Hz, 3H). LC/MS: Rt=4.63 min; m/z=641 [M+H]⁺.

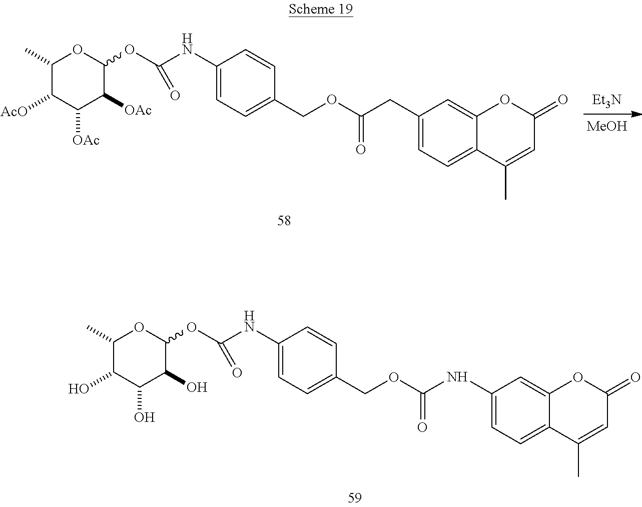 [SMILES: "Et3N" ; "MeOH" ; CC(=O)O[C@@H]1[C@H](OC(C)=O)[C@H](C)OC(OC(=O)Nc2ccc(COC(=O)Cc3ccc4c(C)cc(=O)oc4c3)cc2)[C@H]1OC(C)=O ; Cc1cc(=O)oc2cc(NC(=O)OCc3ccc(NC(=O)OC4O[C@@H](C)[C@@H](O)[C@@H](O)[C@@H]4O)cc3)ccc12]

Scheme 19 illustrates the synthesis of compound 59.

### Example 22: [4-[[(2S,3S,4R,5S,6S)-3,4,5-trihydroxy-6-methyl-tetrahydropyran-2-yl]oxycarbonylamino]phenyl]methyl N-(4-methyl-2-oxo-chromen-7-yl)carbamate

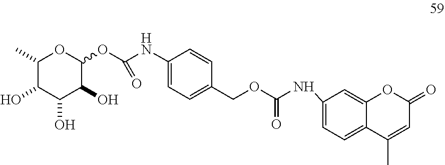 [SMILES: Cc1cc(=O)oc2cc(NC(=O)OCc3ccc(NC(=O)OC4O[C@@H](C)[C@@H](O)[C@@H](O)[C@@H]4O)cc3)ccc12]

To a solution of [rac-(2S,3R,4R,5S,6S)-4,5-diacetoxy-2-methyl-6-[[4-[(4-methyl-2-oxo-chromen-7-yl)carbamoyloxymethyl]phenyl]carbamoyloxy]tetrahydropyran-3-yl] acetate (58) (27 mg, 0.0421 mmol, 1.00 eq) in methyl alcohol (1.25 mL) was added water (0.175 mL) and triethylamine (0.059 mL, 0.421 mmol, 10.0 eq). The reaction mixture was stirred at room temperature for 24 h. The reaction mixture was concentrated and purified by reverse phase chromatography C18 column, eluted with water/acetonitrile (+0.1% formic acid) (30-50%) to give the title compound (59) (18 mg, 82%) as a white solid. ¹H NMR (400 MHz, DMSO) d 10.25 (s, 1H), 9.71 (s, 1H), 7.70 (d, J=8.7 Hz, 1H), 7.55 (d, J=2.2 Hz, 1H), 7.51 (d, J=8.2 Hz, 2H), 7.43−7.36 (m, 3H), 6.24 (d, J=1.2 Hz, 1H), 5.90 (d, J=3.7 Hz, 1H), 5.12 (s, 2H), 5.00 (d, J=5.0 Hz, 1H), 4.73 (d, J=5.3 Hz, 1H), 4.64−4.60 (m, 1H), 3.98−3.94 (m, 1H), 3.79−3.67 (m, 2H), 3.58−3.55 (m, 1H), 2.39 (d, J=1.2 Hz, 3H), 1.10 (d, J=6.5 Hz, 3H).

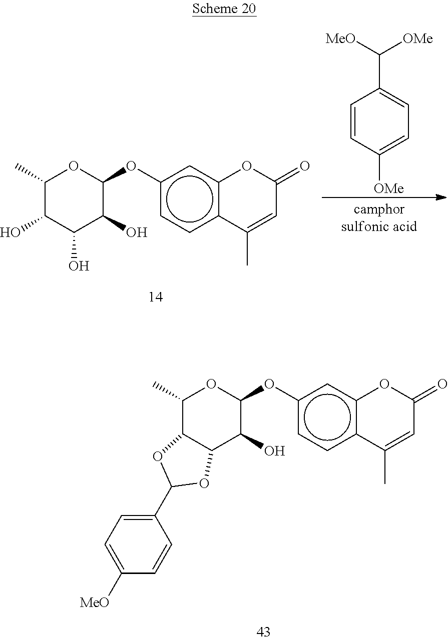

### Example 23: 7-(((3aR,4S,6S,7S,7aS)-7-hydroxy-2-(4-methoxyphenyl)-4-methyltetrahydro-3aH-[1,3]dioxolo[4,5-c]pyran-6-yl)oxy)-4-methyl-2H-chromen-2-one (43)

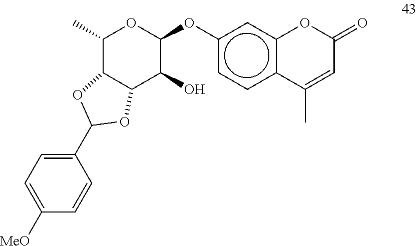

To a stirred suspension of 4-methyl-7-(((2S,3S,4R,5S,6S)-3,4,5-trihydroxy-6-methyltetrahydro-2H-pyran-2-yl)oxy)-2H-chromen-2-one (80 mg, 0.248 mmol) (14) in acetonitrile at room temperature was added 4A° molecular sieves (10 mg) followed by 1-(dimethoxymethyl)-4-methoxybenzene (0.135 mL, 0.745 mmol). The mixture was stirred at room temperature for 30 min. Then camphor sulfonic acid (10 mg, 0.037 mmol) was added and the reaction mixture was stirred at room temperature for 16 h. The solid was then filtered off and the organic solvent was concentrated in vacuo and the residue was purified by HPLC to afford 7-(((3aR,4S,6S,7S,7aS)-7-hydroxy-2-(4-methoxyphenyl)-4-methyltetrahydro-3aH- [1,3]dioxolo[4,5-c]pyran-6-yl)oxy)-4-methyl-2H-chromen-2-one (43) (16.3 mg, 11%yield). ¹H NMR (500 MHz, DMSO-d6) δ 7.74 (d, J=8.6 Hz, 1H), 7.44 (d, J=8.2 Hz, 1H), 7.38 (d, J=8.2 Hz, 1H), 7.09 (dq, J=13.0, 3.1, 3.1, 2.5 Hz, 2H), 7.02−6.91 (m, 2H), 6.25 (d, J=1.4 Hz, 1H), 5.96 (d, J=116.6 Hz, 1H), 5.71−5.55 (m, 2H), 4.64−4.32 (m, 1H), 4.24−4.13 (m, 2H), 3.77 (dd, J=7.4, 1.2 Hz, 3H), 2.40 (d, J=1.5 Hz, 3H), 1.19 (dd, J=16.8, 6.1 Hz, 3H).

### Example 24: Procedure for Cellular Hydrolysis of Compounds

T47D breast cancer cells were cultured in RPMI 1640 medium containing 10% heat inactivated fetal bovine serum. Cell lines were infected with lentiviral construct(s) containing S. pyogenes Cas9 and sgRNA(s) targeting the gene(s) of interest (sgNTC, non-targeting control; sgFUCA1_1, FUCA1 knockout). Infected cells were selected by antibiotic treatment. To assess the cellular hydrolysis of compounds, cells were seeded at 3-20,000 cells per well into 96-well plates and incubated at 37° C. with 5% CO₂. The next day, compounds (30 uM) were added to the cells. Wells containing only compounds in media were included as background fluorescence controls. 4-methylumbelliferyl-alpha-L-fucopyranoside (referred to as MU-Fuc, CAS 54322-38-2) or 4-methylumbelliferyl-2,3,4,6-tetra-O-acetyl-beta-D-galactopyranoside (CAS 6160-79-8) were included as positive controls. A 4-methylumbelliferone (referred to as 4-MU, CAS 90-33-5) standard ladder ranging from 39 to 5000 pmoles was added to each cell line. Fluorescence was read at the excitation wavelength of 330 nm and the emission wavelength of 450 nm on the plate reader every 24 hours for 72 hours. The plate was returned to the 37° C. 5% CO2 incubator between readings. A linear regression was performed to determine the slope of the line relating fluorescence to pmoles of 4-MU standard at each time point. The average cell-free background fluorescence for each compound at each time point was subtracted from all compound sample fluorescence measurements. Compound sample fluorescence was converted to pmoles of 4-MU by dividing by the slope of the 4-MU linear regression. The amount of 4-MU present at 72 hours for each compound in each cell line was compared as a percentage of the appropriate positive control compound.

### Example 25: Procedure for Enzymatic Hydrolysis of Galactose and Fucose Compounds

Compounds were added to wells of a black-walled clear bottom 96 well plate at a concentration of 500 μM284801. 4-methylumbelliferyl-alpha-L-fucopyranoside (referred to as MU-Fuc, CAS 54322-38-2) or 4-methylumbelliferyl-beta-D-galactopyranoside (referred to as MU-Gal, CAS 6160-78-7) were included as positive control compounds. Recombinant human FUCA1 (1-25 nM) or GLB1 (2-50 nM) in reaction buffer comprised of pH 4.4 McIlvaine Buffer, 2 mM dithiothreitol and 0.1% (v/v) Tween-20 was added to the wells. Enzyme-free controls were included to check for spontaneous compound hydrolysis. The plate was shaken and incubated at 37° C. in the plate reader. Every 5 minutes, fluorescence was read at the excitation wavelength of 330 nm and the emission wavelength of 450 nm for up to 3 hours. Linear regressions were calculated during the linear phase of the reaction to calculate the reaction velocity at each concentration of compound. Reaction velocities for compounds at 500 uM were compared as a percentage of the appropriate positive control compound.

### Example 26: Hydrolysis by the Glb1 Enzyme of Modified Galactose Sugars Conjugated to 4-Methylumbelliferone

TABLE 1

| | | | Average percent |
|---|---|---|---|
| | | rhGLB1 | of MU-Gal reaction |
| Modification | Compound # | substrate | velocity |
| none | | yes | 100 |
| 2-deoxy | 24 | no | 0 |
| 3-deoxy | 35 | yes | 1.4 |
| 4-deoxy | 33 | yes | 4.4 |
| 6-deoxy | 14 | yes | 7.6 |
| 2-deoxy-2-fluoro | 20 | no | 0 |
| 3-deoxy-3-fluoro | 31 | yes | 0.8 |
| 4-deoxy-4-fluoro | 27 | yes | 0.4 |
| 6-deoxy-6-fluoro | 6 | yes | 2.7 |
| 6-deoxy-6,6-difluoro | 4 | no | 0 |

Hydrolysis by the recombinant human GLB1 (rhGLB1) enzyme of modified galactose sugars conjugated to 4-methylumbelliferone was assessed by quantifying the generation of fluorescence over time as described in Example 10 and is compiled in Table 1, supra. Reaction rates of each compound were compared to the hydrolysis rate of the unmodified galactose conjugate MU-Gal. Of the modified galactose compounds tested, compound 42 was rapidly hydrolyzed, at a rate about 8% of that of MU-Gal. Compounds 33 and 6 were hydrolyzed with reaction velocities about 4% or 3% of that of MU-Gal, respectively. Compounds 35, 31, and 27 were hydrolyzed by rhGLB1, but slowly compared to MU-Gal with rates less than 2% of that of MU-Gal. Compounds 24, 20, and 4 were not hydrolyzed by rhGLB1, indicating that they were not suitable substrates for the enzyme under the conditions of Example 10.

### Example 27: Hydrolysis by the FUCA1 Enzyme of Modified Fucose Sugars Conjugated to 4-methylumbelliferone or 7-amino-4-methylcoumarin

TABLE 2

| | | | Average reaction |
|---|---|---|---|
| | | rhFUCA1 | velocity relative |
| Modification | Compound | substrate | to MU-Fuc (%) |
| none | MU-Fuc | yes | 100 |
| | (14) | | |
| 2-deoxy | 19 | yes | 29.6 |
| 4-methoxybenzylidene | 43 | yes | 35.2 |
| acetal | | | |
| 4-hydroxy benzyloxy | 50 | yes | 16.7 |
| carbamate | | | |
| 4-amino benzyloxy | 59 | yes | 77 |
| carbamate | | | |

Hydrolysis by the recombinant human FUCA1 enzyme (rhFUCA1) of modified fucose sugars conjugated to 4-methylumbelliferone or 7-amino-4-methylcoumarin was assessed by quantifying the generation of fluorescence over time as described in Example 10 and is compiled in Table 1, supra. Reaction rates of each compound were compared to the hydrolysis of the unmodified fucose conjugate MU-Fuc (14). Compound 43 was hydrolyzed by rhFUCA1 at a velocity about 35% of that of MU-Fuc. The reaction rate of 19 with rhFUCA1 was about 30% of that of MU-Fuc. Notably the reaction rate of 59 was about 77% of that of MU-Fuc.

### Example 28: Hydrolysis in Live Cells of Modified Galactose Sugars Conjugated to 4-Methylumbelliferone

TABLE 3

| | Average percent of product relative to 26 at | | | |
|---|---|---|---|---|
| | 72 hours in T47D control cells | | | |
| | Naked | | | |
| Modification | sugar | Acetylated version | | |
| none | | | 26 | 100 |
| 2-deoxy | 24 | 11.5 | 25 | 6 |
| 3-deoxy | 35 | 49 | 37 | 23 |
| 4-deoxy | 33 | 96 | 34 | 80.5 |
| 6-deoxy | 14 | 97 | n/a | n/a |
| 2-deoxy-2-fluoro | 20 | 0 | 42 | 3 |
| 3-deoxy-3-fluoro | 31 | 31.5 | 32 | 50.5 |
| 4-deoxy-4-fluoro | 27 | 13 | 30 | 73.5 |
| 6-deoxy-6-fluoro | 6 | 114 | 41 | 27 |
| 6-deoxy-6,6-difluoro | 4 | 4 | 29 | 1 |

The hydrolysis of modified galactose sugars conjugated to 4-methylumbelliferone in live cells was assessed by incubating the compounds in media with T47D breast cancer cells for 3 days and quantifying the generation of fluorescence over time as described in Example 11 and is compiled in Table 3, supra. The amount of product formed by each compound after 3 days was compared to the tetra-acetylated galactose conjugate 26. Compounds 35, 42 and 6 were hydrolyzed similarly in the live cells as compound 26. Compounds 4 and 31 formed about 74% and 80% product as compound 26, respectively. Compounds 32 and 30 each formed about 50% of the product compared to compound 26. The other compounds tested formed only a third of the amount of product as compound 26 or less in T47D cells.

### Example 29: Hydrolysis in Live Cells of Modified Fucose Sugars Conjugated to 4-methylumbelliferone or 7-amino-4-methylcoumarin

TABLE 4

| | Average percent of product relative to MU-Fuc (14) | | | |
|---|---|---|---|---|
| | at 72 hours by T47D control cells | | | |
| | Naked | | | |
| Modification | sugar | Acetylated version | | |
| none | MU-Fuc | 100 | 44 | 17.6 |
| | (14) | | | |
| 2-deoxy | 19 | 99 | 18 | 17.3 |
| 4-methoxy- | 43 | 6.1 | n/a | n/a |
| benzylidene | | | | |
| acetal | | | | |
| 4-hydroxy | 50 | n/a | 51 | 25 |
| benzyloxy | | | | |
| carbamate | | | | |
| 4-amino | 59 | n/a | 58 | 23.6 |
| benzyloxy | | | | |
| carbamate | | | | |

The hydrolysis of modified fucose compounds conjugated to 4-methylumbelliferone or 7-amino-4-methylcoumarin in live cells was assessed by incubating the compounds in media with T47D breast cancer cells for 3 days and quantifying the generation of fluorescence over time as described in Example 11 and is compiled in Table 4, supra. The amount of product formed by each compound after 3 days was compared to the unmodified fucose conjugate MU-Fuc (14). The hydrolysis of compound 19 in live cells was very similar to that of MU-Fuc. The acetylated versions of compounds 14 and 19, 44 and 18 respectively, performed similarly to one another as well, each generating about 17% of product compared to MU-Fuc. Compounds 51 and 58 performed slightly better. Compound 43 generated about 6% of the product generated by MU-Fuc in T47D cells.

### Example 30: Hydrolysis of Modified Fucose Sugars Conjugated to 4-methylumbelliferone or 7-amino-4-methylcoumarin in FUCA1-Proficient (Control Cells) Versus FUCA1-Deficient Cells

TABLE 5

| | Average percent of product formed by each compound | | | |
|---|---|---|---|---|
| | at 72 hours in T47D FUCA1 knockout cells relative | | | |
| | to product formed in T47D control cells | | | |
| | Naked | | | |
| Modification | sugar | Acetylated version | | |
| none | MU-Fuc | 5.4 | 44 | 8 |
| | (14) | | | |
| 2-deoxy | 19 | 2.9 | 18 | 12.4 |
| 4-methoxy- | 43 | 5.7 | n/a | n/a |
| benzylidene | | | | |
| acetal | | | | |
| 4-hydroxy | 50 | | 51 | 44.7 |
| benzyloxy | | | | |
| carbamate | | | | |
| 4-amino | 59 | n/a | 58 | 48.7 |
| benzyloxy | | | | |
| carbamate | | | | |

The hydrolysis of modified fucose compounds conjugated to 4-methylumbelliferone or 7-amino-4-methylcoumarin in FUCA1 proficient or deficient cells was assessed by incubating the compounds in media with T47D control cells or FUCA1 knockout cells for 3 days as described in Example 11 and is compiled in Table 5, supra. The ratio of product formed after 3 days by each compound in the FUCA1 proficient or deficient cells was determined. Only about 5% of the total product formed by MU-Fuc in control cells after 3 days was generated in FUCA1 knockout cells, demonstrating that FUCA1 fucosidase activity is necessary for the hydrolysis of MU-Fuc in live cells. The acetylated version of MU-Fuc, compound 44, formed 8% of the product generated in control cells in the FUCA1 deficient cells. Only about 3% of the product formed by compound 19 in control cells was generated in FUCA1 knockout cells after 3 days. The acetylated version of compound 19, compound 18, formed about 12% of the product formed in control cells in the FUCA1 knockout cells. FUCA1 knockout cells incubated with compound 43 generated about 6% of the product generated by the compound in control cells. Compounds 51 and 58 performed much better than other acetylated products. All fucose conjugates tested demonstrated reliance on cellular FUCA1 fucosidase activity for hydrolysis in live cells.

What is claimed is:

1. A compound of Formula (I) or Formula (II): 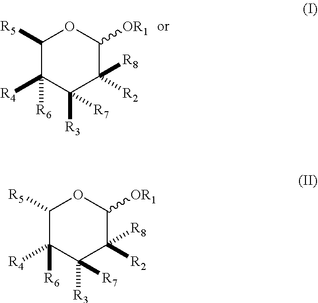
or pharmaceutically available salts, hydrates and solvates thereof, wherein:
R₁ is 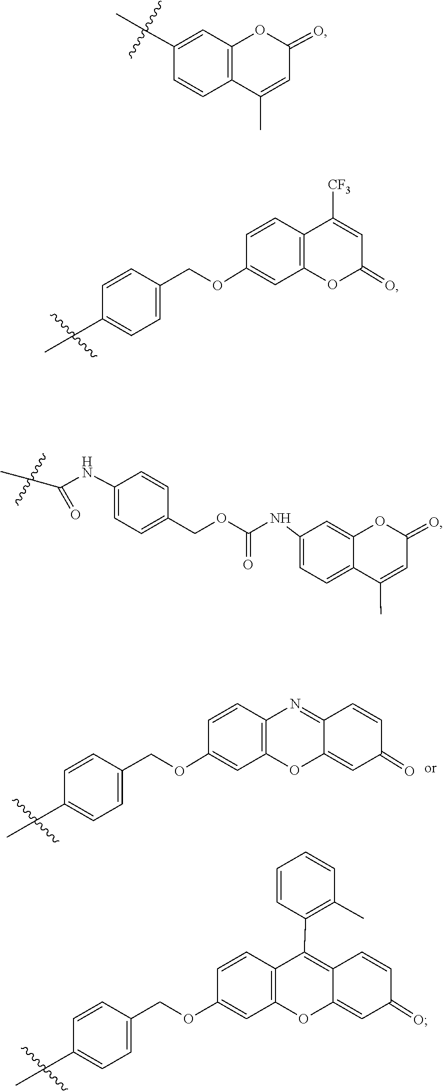 [SMILES: *c1ccc2c(C)cc(=O)oc2c1 ; *c1ccc(COc2ccc3c(C(F)(F)F)cc(=O)oc3c2)cc1 ; *C(=O)Nc1ccc(COC(=O)Nc2ccc3c(C)cc(=O)oc3c2)cc1 ; *c1ccc(COc2ccc3nc4ccc(=O)cc-4oc3c2)cc1 ; *c1ccc(COc2ccc3c(-c4ccccc4C)c4ccc(=O)cc-4oc3c2)cc1]
R₂ is —H, —F, —OH, —OC(O)R₉ or —OC(O)OR₁₀;
R₃ is —H, —F, —OH, —OC(O)R₁₁ or —OC(O)OR₁₂;
R₄ is —H, —F, —OH, —OC(O)R₁₃ or —OC(O)OR₁₄;
alternatively, both R₃ and R₄ together with the atoms to which they are bonded form a 5 membered cyclic acetal which is substituted by R₁₇ at the acetal carbon atom;
alternatively, both R₃ and R₄ together with the atoms to which they are bonded form a 5 membered cyclic carbonate;
R₅ is —CH₃, —CH₂F, —CHF_{2,} —CF₃, —CH₂OH, —CH₂OC(O)R₁₅ or —CH₂OC(O)OR₁₆;
R₆ is —H or —F;
R₇ is —H or —F;
R₈ is —H or —F; and
R₉-R₁₇ are independently alkyl, substituted alkyl, alkenyl, substituted alkenyl, alkynyl, substituted alkynyl, aryl, substituted aryl, cycloalkyl, substituted cycloalkyl, cycloheteroalkyl, substituted cycloheteroalkyl, heteroaryl or substituted heteroaryl;
provided that when R₅ is —CH₂F, —CHF₂ or —CF₃, then one of R₂, R₃ or R₄ is —H or —F;
provided that when R₅ is —CH_{3,} —CH₂OH, —CH₂OC(O)R₁₅ or —CH₂OC(O)OR₁₆, then one or two of R_{2,} R₃ or R₄ is —H or —F;
provided that R₆ is —F only if R₄ is —F; R₇ is —F only if R₃ is —F; and R₈ is —F only if R₂ is —F;
provided R₂ and R₄ are not —F and R₅ is not —CH_{3.}

2. The compound of claim 1, wherein R₂ is —H or —F and R₃ is —H or —F.

3. The compound of claim 1, wherein R₂ is —H or —F and R₄ is —H or —F.

4. The compound of claim 1, wherein R₃ is —H or —F and R₄ is —H or —F.

5. The compound of claim 1, wherein R₂ is —H or —F, R₃ is —F and R₇ is —F.

6. The compound of claim 1, wherein R₂ is —H or —F, R₄ is —F and R₆ is —F.

7. The compound of claim 1, wherein R₃ is —H or —F, R₄ is —F and R₆ is —F.

8. The compound of claim 1, wherein R₂ is —F, R₈ is —F and R₃ is —H or —F.

9. The compound of claim 1, wherein R₂ is —F, R₈ is —F and R₄ is —H or—F.

10. The compound of claim 1, wherein R₃ is —F, R₇ is —F and R₄ is —H or —F.

11. The compound of claim 1, wherein R₃ is —F, R₇ is —F and R₂ is —H or —F.

12. The compound of claim 1, wherein R₂ is —F and R₈ is —F.

13. The compound of claim 1, wherein R₃ is —F and R₇ is —F.

14. The compound of claim 1, wherein R₄ is —F and R₆ is —F.

15. The compound of claim 1, wherein R₂ is —H or —F.

16. The compound of claim 1, wherein R₃ is —H or —F.

17. The compound of claim 1, wherein R₄ is —H or —F.

18. The compound of claim 1, wherein R₉-R₁₇ are independently alkyl, alkenyl, alkynyl, aryl, substituted aryl, cycloalkyl, cycloheteroalkyl or heteroaryl.

19. A diagnostic composition comprising a diagnostically effective of a compound of claim 1 and a diagnostically acceptable vehicle.

20. A method of measuring the rate of hydrolysis of a compound of claim 1 comprising adding a glycoside hydrolase to the diagnostic composition of claim 19.
